(19) United States
(12) Patent Application Publication
(10) Pub. No.: US 2007/0254910 A1
(43) Pub. Date: Nov. 1, 2007

(54) CHEMICAL COMPOUNDS

(76) Inventor: Kristjan GUDMUNDSSON, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/574,586

(22) PCT Filed: Aug. 31, 2005
(86) PCT No.: PCT/US05/31099

§ 371 Date: Mar. 2, 2007

Related U.S. Application Data
(60) Provisional application No. 60/606,742, filed on Sep. 2, 2004.
(60) Provisional application No. 60/610,765, filed on Sep. 17, 2004.

(51) Int. Cl. C07D 471/02; A61K 31/4745; A61K 31/655

(52) U.S. Cl. 514/303; 546/119; 514/151

(57) ABSTRACT

The present invention provides novel compounds that demonstrate protective effects on target cells from HIV infection in a manner as to bind to a chemokine receptor, and which affect the binding of the natural ligand or chemokine to a receptor such as CXCR4 of a target cell.

### FIELD OF THE INVENTION

The present invention provides novel compounds that demonstrate protective effects on target cells from HIV infection in a manner as to bind specifically to the chemokine receptor, and which affect the binding of the natural ligand or chemokine to a receptor such as CXCR4 and/or CCR5 of a target cell.

### BACKGROUND OF THE INVENTION

HIV gains entry into host cells by means of the CD4 receptor and at least one co-receptor expressed on the surface of the cell membrane. M-tropic strains of HIV utilize the chemokine receptor CCR5, whereas T-tropic strains of HIV mainly use CXCR4 as the co-receptor. HIV co-receptor usage largely depends on hyper-variable regions of the V3 loop located on the viral envelope protein gp120. Binding of gp120 with CD4 and the appropriate co-receptor results in a conformational change and unmasking of a second viral envelope protein called gp41. The protein gp41 Subsequently interacts with the host cell membrane resulting in fusion of the viral envelop with the cell. Subsequent transfer of viral genetic information into the host cell allows for the continuation of viral replication. Thus infection of host cells with HIV is usually associated with the virus gaining entry into the cell via the formation of the ternary complex of CCR5 or CXCR4, CD4, and gp120.

A pharmacological agent that would inhibit the interaction of gp120 with either CCR5/CD4 or CXCR4/CD4 would be a useful therapeutic in the treatment of a disease, disorder, or condition characterized by infection with M-tropic or T-tropic strains, respectively, either alone or in combination therapy.

Evidence that administration of a selective CXCR4 antagonist could result in an effective therapy comes from in vitro studies that have demonstrated that addition of ligands selective for CXCR4 as well as CXCR4-neutralizing antibodies to cells can block HIV viral/host cell fusion. In addition, human studies with the selective CXCR4 antagonist AMD-3100, have demonstrated that such compounds can significantly reduce T-tropic HIV viral load in those patients that are either dual tropic or those where only the T-tropic form of the virus is present.

In addition to serving as a co-factor for HIV entry, it has been recently suggested that the direct interaction of the HIV viral protein gp120 with CXCR4 could be a possible cause of CD8+ T-cell apoptosis and AIDS-related dementia via induction of neuronal cell apoptosis.

The signal provided by SDF-1 on binding to CXCR4 may also play an important role in tumor cell proliferation and regulation of angiogenesis associated with tumor growth; the known angiogenic growth factors VEG-F and bFGF up-regulate levels of CXCR4 in endothelial cells and SDF-1 can induce neovascularization in vivo. In addition, leukemia cells that express CXCR4 migrate and adhere to lymph nodes and bone marrow stromal cells that express SDF-1.

The binding of SDF-1 to CXCR4 has also been implicated in the pathogenesis of atherosclerosis, renal allograft rejection, asthma and allergic airway inflammation, Alzheimer's disease, and arthritis.

The present invention is directed to compounds that can act as agents that modulate chemokine receptor activity. Such chemokine receptors include, but are not limited to, CCR1, CCR2, CCR3, CCR4, CCR5, CCR6, CCR7, CCR8, CXCR1, CXCR2, CXCR3, CXCR4, and CXCR5.

The present invention provides novel compounds that demonstrate protective effects on target cells from HIV infection in a manner as to bind specifically to the chemokine receptor, and which affect the binding of the natural ligand or chemokine to a receptor, such as CXCR4 and/or CCR5 of a target cell.

### SUMMARY OF THE INVENTION

The present invention includes compounds of formula (I): 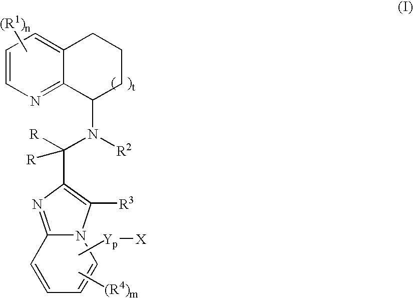
including salts, solvates, and physiologically functional derivatives thereof, wherein:
t is 0, 1, or 2;
each R independently is H, alkyl, alkenyl, alkynyl, haloalkyl, cycloalkyl, —R^{a}Ay, —R^{a}OR¹⁰, or —R^{a}S(O)_{q}R¹⁰;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁸R⁷, R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2, such that an R¹ may be substituted throughout the depicted tetrahydroquinoline ring;
R² is selected from a group consisting of H, alkyl, haloalkyl, cycloalkyl, alkenyl, alkynyl, R^{a}Ay, R^{a}OR⁵, —R^{a}S(O)_{q}R⁵;
R³ is H, alkyl, haloalkyl, cycloalkyl, alkenyl, alkynyl, —R^{a}Ay, —R^{a}OR⁵, or —R^{a}S(O)_{q}R⁵;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —C(O)NR¹⁰—, —NR¹⁰C(O)—, —C(O)—, —C(O)O—, —NR¹⁰C(O)N(R¹⁰)—, S(O)_{q}—, S(O)_{q}NR¹⁰, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay or -HetR^{a}Het;
each R^{a} independently is alkylene, cycloalkylene, alkenylene, cycloalkenylene, or alkynylene;
each R¹⁰ independently is H, alkyl, cycloalkyl, alkenyl, alkynyl, cycloalkenyl, R^{a}cycloalkyl, —R^{a} OH, —R^{a}OR⁵, —R^{a}NR⁶R⁷, or R^{a}Het
each of R⁶ and R⁷ independently are selected from H, alkyl, alkenyl, alkynyl, cycloalkyl, cycloalkenyl, R^{a}cycloalkyl, —R^{a} OH, —R^{a}OR⁵, R^{a}NR⁸R⁹, -Ay, -Het, R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted 4-, 5-, or 6-membered heterocyclyl or heteroaryl group.

Preferably t is 1.

In one embodiment R is H or alkyl. Preferably R is H.

In one embodiment n is 0.

In one embodiment n is 1 and R¹ is halogen, haloalkyl, alkyl, OR¹⁰, NR⁶R⁷, CO₂R¹⁰, CONR⁶R⁷, or cyano.

In one embodiment R² is H, alkyl, haloalkyl, or cycloalkyl. Preferably R² is alkyl, haloalkyl, or cycloalkyl.

In one embodiment R³ is H, alkyl, haloalkyl, cycloalkyl, alkenyl, or alkynyl. Preferably R³ is H, alkyl, haloalkyl, or cycloalkyl. More preferably R³ is H or alkyl. More preferably R³ is H.

In one embodiment m is 0.

In one embodiment m is 1 or 2. Preferably m is 1.

When m is not 0, R⁴ preferably is one or more of halogen, haloalkyl, alkyl, OR¹⁰, NR⁶R⁷, CO₂R¹⁰, CONR⁶R⁷, or cyano.

In one embodiment p is 0 and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂. Preferably X is —R^{a}N(R¹⁰)₂, -Het, —R^{a}Het, -HetN(R¹⁰)₂, —R^{a}HetN(R¹⁰)₂, or -HetR^{a}N(R¹⁰)₂. More preferably X is R^{a}N(R¹⁰)₂, -Het, —R^{a}Het, or -HetN(R¹⁰)₂.

In one embodiment p is 1; Y is —N(R¹⁰)—, —O—, —S—, —CONR¹⁰—, —NR¹⁰CO—, or —S(O)_{q}NR¹⁰—; and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂. Preferably Y is —N(R¹⁰)—, —O—, —CONR¹⁰—, —NR¹⁰CO— and X is —R^{a}N(R¹⁰)₂, -Het, —R^{a}Het, or -HetN(R¹⁰)₂,

Preferably Het is piperidine, piperazine, azetidine, pyrrolidine, imidazole, pyridine, and the like.

In one embodiment p is 0 and X is -Het. Preferably -Het is unsubstituted or substituted with one or more C₁-C₆ alkyl or C₃-C₈ cycloalkyl.

Preferably the substituent —Yₚ—X is located on the depicted imidazopyridine ring as in formula (I′):

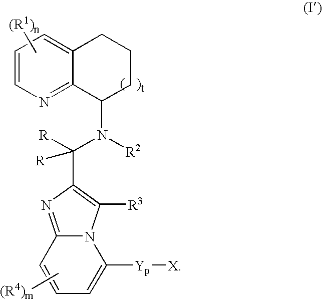

One aspect further includes compounds wherein R³ is R^{a}OR⁵ as shown in formula (I-G): 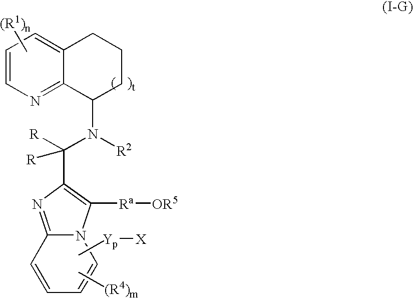
wherein
t is 0, 1, or 2;
each R independently is H, alkyl, alkenyl, alkynyl, haloalkyl, cycloalkyl, —R^{a}Ay, —R^{a}OR¹⁰, or R^{a}S(O)_{q}R¹⁰;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay C(O)Het, —S(O)₂NR⁶R⁷, —S(O)_{q}R¹⁰, —S(O)_{q}Ay, cyano, nitro, or azido;
n is 0, 1, or 2, such that an R¹ may be substituted throughout the depicted tetrahydroquinoline ring;

R² is selected from a group consisting of H, alkyl, haloalkyl, cycloalkyl, —R^{a}cycloalkyl, alkenyl, alkynyl, —R^{a}Ay —R^{a}OR⁵, —R^{a}S(O)_{q}R⁵; wherein R² is not substituted with amine or alkylamine

each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, R^{a}C(O)R¹⁰, —C(O)R¹⁰, —CO₂R¹⁰, —R^{a}CO₂R¹⁰, —C(O)NR⁶R⁷, C(O)Ay, C(O)Het, —S(O)₂NR⁶R⁷, —S(O)_{q}R¹⁰, —S(O)_{q}Ay, cyano, nitro, or azido;

m is 0, 1, or 2;

each R⁵ independently is H, alkyl, alkenyl, alkynyl, cycloalkyl, or -Ay;

p is 0 or 1;

Y is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, —S(O)_{q}NR¹⁰—, or —NR¹⁰S(O)_{q}—;

X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay or -HetR^{a}Het;

each R^{a} independently is alkylene optionally substituted with one or more of alkyl, oxo or hydroxyl, cycloalkylene optionally substituted with one or more of alkyl, oxo or hydroxyl, alkenylene, cycloalkenylene, or alkynylene;

each R¹⁰ independently is H, alkyl, cycloalkyl, alkenyl, alkynyl, cycloalkenyl, —R^{a}cycloalkyl, —R^{a}OH, —R^{a}OR⁶, —R^{a}NR⁶R⁷, or —R^{a}Het

each of R⁶ and R⁷ independently are selected from H, alkyl, alkenyl, alkynyl, cycloalkyl, cycloalkenyl, R^{a}cycloalkyl, —R^{a} OH, —R^{a}OR⁵, —R^{a}NR⁸R⁹, -Ay, -Het, —R^{a}Ay, —R^{a}Het, or —S(O)_{q}R⁵;

each of R⁸ and R⁹ independently are selected from H or alkyl;

each q independently is 0, 1, or 2;

each Ay independently represents an optionally substituted aryl group; and

each Het independently represents an optionally substituted heterocyclyl or heteroaryl group; or pharmaceutically acceptable salts or esters thereof.

In one aspect of the invention, the substituent —Yₚ—X is located on the depicted imidazopyridine ring as in formula (I-G′): 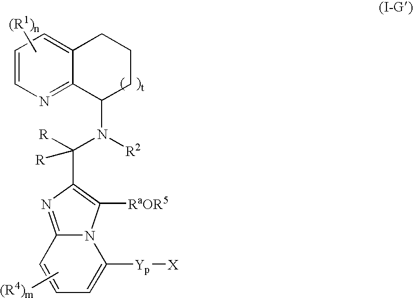
wherein all substituents are as defined with respect to formula (I-G), or pharmaceutically acceptable salts or esters thereof.

One aspect of the invention includes compounds of formula (I-G) where -Het is optionally substituted with at least one of alkyl, alkoxy, hydroxyl, halogen, haloalkyl, cycloalkyl, cycloalkoxy, cyano, amide, amino, or alkylamino. In yet another embodiment, -Het is substituted with at least one of C₁-C₆ alkyl or C₃-C₈ cycloalkyl. One aspect of the invention includes compounds of formula (I-G) where -Ay is optionally substituted with at least one of alkyl, alkoxy, hydroxyl, halogen, haloalkyl, cycloalkyl, cycloalkoxy, cyano, amide, amino, or alkylamino. In yet another embodiment, -Ay is substituted with at least one of C₁-C₆ alkyl or C₃-C₈ cycloalkyl.

One aspect of the invention includes compounds of formula (I-G) where t is 1. One aspect of the invention includes compounds of formula (I-G) where t is 2.

One aspect of the invention includes compounds of formula (I-G) where R is H, alkyl, cycloalkyl, or R^{a}OR¹⁰. One aspect of the invention includes compounds of formula (I-G) where R is H or alkyl. One aspect of the invention includes compounds of formula (I-G) where R is H.

One aspect of the invention includes compounds of formula (I-G) where n is 0. One aspect of the invention includes compounds of formula (I-G) where n is 1 and R¹ is halogen, haloalkyl, alkyl, OR¹⁰, NR⁶R⁷, CO₂R¹⁰, CONR⁶R⁷, or cyano.

One aspect of the invention includes compounds of formula (I-G) where R² is H, alkyl, haloalkyl, R^{a}OR⁵ or R^{a}cycloalkyl. One aspect of the invention includes compounds of formula (I-G) where R² is H, alkyl or R-cycloalkyl. One aspect of the invention includes compounds of formula (I-G) where R² is alkyl. One aspect of the invention includes compounds of formula (I-G) where R² is R^{a}Ay or R^{a}cycloalkyl.

One aspect of the invention includes compounds of formula (I-G) where R^{a} is alkylene optionally substituted with C₁-C₆alkyl and R⁵ is H, alkyl or cycloalkyl. Another aspect of the invention includes compounds of formula (I-G) where R^{a} is methylene (—CH₂—) optionally substituted with C₁-C₆alkyl and R⁵ is H, alkyl or cycloalkyl. One aspect of the invention includes compounds of formula (I-G) where R^{a} is methylene (—CH₂—) and R⁵ is H, or alkyl. In yet another aspect of the invention, compounds of formula (I-G) are provided where R^{a} is methylene and R⁵ is H.

One aspect of the invention includes compounds of formula (I-G) where m is 0. One aspect of the invention includes compounds of formula (I-G) where m is 1 or 2. One aspect of the invention includes compounds of formula (I-G) where m is 1. One aspect of the invention includes compounds of formula (I-G) where m is 1 and R⁴ is halogen, haloalkyl, alkyl, OR¹⁰, NR⁶R⁷, CO₂R¹⁰, CONR⁶R⁷, or cyano.

One aspect of the invention includes compounds of formula (I-G) where p is 0 and X is R^{a}N(R¹⁰)₂, AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂. One aspect of the invention includes compounds of formula (I-G) where p is 0 and X is —R^{a}N(R¹⁰)₂, -Het, —R^{a}Het, -HetN(R¹⁰)₂, —R^{a}HetN(R¹⁰)₂, or -HetR^{a}N(R¹⁰)₂. One aspect of the invention includes compounds of formula (I-G) where p is 0 and X is —R^{a}N(R¹⁰)₂, -Het, —R^{a}Het, or -HetN(R¹⁰)₂.

One aspect of the invention includes compounds of formula (I-G) where p is 1; Y is —N(R¹⁰)—, —O—, —S—, —C(O)NR¹⁰—, —NR¹⁰C(O)—, or —S(O)_{q}NR¹⁰—; and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂. One aspect of the invention includes compounds of formula (I-G) where p is 1; Y is —N(R¹⁰)—, —O—, —C(O)NR¹⁰—, or —NR¹⁰C(O)—; and X is —R^{a}N(R¹⁰)₂, -Het, —R^{a}Het, or -HetN(R¹⁰)₂. One aspect of the invention includes compounds of formula (I-G) where p is 1, Y is —N(R¹⁰)— and X is -Het, unsubstituted or substituted with C₁-C₆ alkyl or C₃-C₈ cycloalkyl.

One aspect of the invention includes compounds of formula (I-G) where t is 1 or 2; R is H or alkyl; R² is H, alkyl, R^{a}cycloalkyl or cycloalkyl; n is 0; and m is 0 and with respect to —R^{a}OR⁵, R^{a} is alkylene optionally substituted with C₁-C₆alkyl and R⁵ is H, alkyl, or cycloalkyl. One aspect of the invention includes compounds of formula (I-G) where t is 1 or 2; R is H or alkyl; R² is H, alkyl, R^{a}cycloalkyl or cycloalkyl; n is 0; m is 0; p is 0 and X is -Het or -HetN(R¹⁰)₂, R¹⁰ is H or alkyl and -Het is unsubstituted or substituted with C₁-C₆ alkyl or C₃-C₈ cycloalkyl and with respect to —R^{a}OR⁵, R^{a} is alkylene optionally substituted with C₁-C₆alkyl and R₅ is H, alkyl, or cycloalkyl. One aspect of the invention includes compounds of formula (I-G) where t is 1 or 2; R is H or alkyl; R² is H, alkyl, R^{a}cycloalkyl or cycloalkyl; n is 0; m is 0; p is 0 and X is -Het or -HetN(R¹⁰)₂, R¹⁰ is H or alkyl and -Het is unsubstituted or substituted with C₁-C₆ alkyl or C₃-C₈ cycloalkyl and —R^{a}OR⁵ is —CH₂OH.

One aspect of the invention includes compounds of formula (I-G) where t is 1 or 2; R is H or alkyl; R² is H, alkyl, R^{a}cycloalkyl or cycloalkyl; n is 0; m is 0; p is 1; Y is —N(R¹⁰)—, —O—, —CONR¹⁰—, or —NR¹⁰CO—; X is -Het or -HetN(R¹⁰)₂, and R¹⁰ is H or alkyl and Het is unsubstituted or substituted with C₁-C₆ alkyl or C₃-C₈ cycloalkyl and with respect to —R^{a}OR₅, R^{a} is alkylene optionally substituted with C₁-C₆alkyl and R₅ is H, alkyl, or cycloalkyl.

One aspect of the invention includes compounds of formula (I-G) where t is 1 or 2; R is H or alkyl; R² is H, alkyl, R^{a}cycloalkyl or cycloalkyl; n is 0; m is 0; p is 1; Y is —N(R¹⁰)— or —O— and X is -Het and wherein with respect to —R^{a}OR⁵, R^{a} is -alkylene optionally substituted with C₁-C₆alkyl and R⁵ is H, alkyl or cycloalkyl.

One aspect of the invention includes compounds of formula (I-G) where t is 1 or 2, R is H or alkyl; R² is H, alkyl, R^{a}cycloalkyl or cycloalkyl; n is 0; and m is 0; p is 0 and X is -Het or -HetN(R¹⁰)₂, R¹⁰ is H or alkyl and Het is unsubstituted or substituted with C₁-C₆ alkyl or C₃-C₈ cycloalkyl.

One aspect of the invention includes compounds of formula (I-G) where p is 0; X is -HetN(R¹⁰)₂; and R¹⁰ is H or alkyl and R^{a} is alkylene optionally substituted with C₁-C₆alkyl and R⁵ is H, alkyl or cycloalkyl. One aspect of the invention includes compounds of formula (I-G) where p is 1 and Y is —N(R¹⁰)—, —O—, —C(O)NR¹⁰—, or —NR¹⁰C(O)—; X is -Het or -HetN(R¹⁰)₂, and Het is unsubstituted or substituted with C₁-C₆ alkyl or C₃-C₈ cycloalkyl and R^{a} is alkylene optionally substituted with C₁-C₆alkyl and R⁵ is H, alkyl or cycloalkyl.

Compounds of the present invention include:
N-Methyl-N-{[5-(4-methyl-1-piperazinyl)imidazo[1,2-a]pyridin-2-yl]methyl}-5,6,7,8-tetrahydro-8-quinolinamine;
N-{[5-(4-Ethyl-1-piperazinyl)imidazo[1,2-a]pyridin-2-yl]methyl}-N-methyl-5,6,7,8-tetrahydro-8-quinolinamine;
N-Methyl-N-({5-[4-(1-methylethyl)-1-piperazinyl]imidazo[1,2-a]pyridin-2-yl}methyl)-5,6,7,8-tetrahydro-8-quinolinamine;
1,1-Dimethylethyl 4-(2-{[methyl(5,6,7,8-tetrahydro-8-quinolinyl)amino]methyl}imidazo[1,2-a]pyridin-5-yl)-1-piperazinecarboxylate;
N-Methyl-N-{[5-(1-piperazinyl)imidazo[1,2-a]pyridin-2-yl]methyl}-5,6,7,8-tetrahydro-8-quinolinamine;
N,N,N′-Trimethyl-N′-(2-{[methyl(5,6,7,8-tetrahydro-8-quinolinyl)amino]methyl}imidazo[1,2-a]pyridin-5-yl)-1,2-ethanediamine;
N-{[5-(3,5-Dimethyl-1-piperazinyl)imidazo[1,2-a]pyridin-2-yl]methyl}-N-methyl-5,6,7,8-tetrahydro-8-quinolinamine;
N-Methyl-N-{[5-(3,4,5-trimethyl-1-piperazinyl)imidazo[1,2-a]pyridin-2-yl]methyl}-5,6,7,8-tetrahydro-8-quinolinamine;
N-(1-Methylethyl)-N-{[5-(4-methyl-1-piperazinyl)imidazo[1,2-a]pyridin-2-yl]methyl}-5,6,7,8-tetrahydro-8-quinolinamine;
N-(1-Methylethyl)-N-({5-[4-(1-methylethyl)-1-piperazinyl]imidazo[1,2-a]pyridin-2-yl}methyl)-5,6,7,8-tetrahydro-8-quinolinamine;
N-{[5-(4-Methyl-1-piperazinyl)imidazo[1,2-a]pyridin-2-yl]methyl}-N-(2,2,2-trifluoroethyl)-5,6,7,8-tetrahydro-8-quinolinamine;
N-({5-[4-(1-Methylethyl)-1-piperazinyl]imidazo[1,2-a]pyridin-2-yl}methyl)-N-(2,2,2-trifluoroethyl)-5,6,7,8-tetrahydro-8-quinolinamine;
N-[(5-{4-[(Dimethylamino)methyl]phenyl}imidazo[1,2-a]pyridin-2-yl)methyl]-N-methyl-5,6,7,8-tetrahydro-8-quinolinamine; and
N-Methyl-N-{[5-(4-pyridinyl)imidazo[1,2-a]pyridin-2-yl]methyl}-5,6,7,8-tetrahydro-8-quinolinamine.

Preferred compounds of the present invention include:
N-Methyl-N-{[5-(4-methyl-1-piperazinyl)imidazo[1,2-a]pyridin-2-yl]methyl}-5,6,7,8-tetrahydro-8-quinolinamine;
N-{[5-(4-Ethyl-1-piperazinyl)imidazo[1,2-a]pyridin-2-yl]methyl}-N-methyl-5,6,7,8-tetrahydro-8-quinolinamine;
N-Methyl-N-({5-[4-(1-methylethyl)-1-piperazinyl]imidazo[1,2-a]pyridin-2-yl}methyl)-5,6,7,8-tetrahydro-8-quinolinamine;
N-Methyl-N-{[5-(1-piperazinyl)imidazo[1,2-a]pyridin-2-yl]methyl}-5,6,7,8-tetrahydro-8-quinolinamine;

N,N,N′-Trimethyl-N′-(2-{[methyl(5,6,7,8-tetrahydro-8-quinolinyl)amino]methyl}imidazo[1,2-a]pyridin-5-yl)-1,2-ethanediamine;
N-{[5-(3,5-Dimethyl-1-piperazinyl)imidazo[1,2-a]pyridin-2-yl]methyl}-N-methyl-5,6,7,8-tetrahydro-8-quinolinamine;
N-Methyl-N-{[5-(3,4,5-trimethyl-1-piperazinyl)imidazo[1,2-a]pyridin-2-yl]methyl}-5,6,7,8-tetrahydro-8-quinolinamine;
N-(1-Methylethyl)-N-{[5-(4-methyl-1-piperazinyl)imidazo[1,2-a]pyridin-2-yl]methyl}-5,6,7,8-tetrahydro-8-quinolinamine;
N-(1-Methylethyl)-N-({5-[4-(1-methylethyl)-1-piperazinyl]imidazo[1,2-a]pyridin-2-yl}methyl)-5,6,7,8-tetrahydro-8-quinolinamine; and
N-[(5-{4-[(Dimethylamino)methyl]phenyl}imidazo[1,2-a]pyridin-2-yl)methyl]-N-methyl-5,6,7,8-tetrahydro-8-quinolinamine.

More preferred compounds of the present invention include:
N-Methyl-N-{[5-(4-methyl-1-piperazinyl)imidazo[1,2-a]pyridin-2-yl]methyl}-5,6,7,8-tetrahydro-8-quinolinamine;
N-{[5-(4-Ethyl-1-piperazinyl)imidazo[1,2-a]pyridin-2-yl]methyl}-N-methyl-5,6,7,8-tetrahydro-8-quinolinamine;
N-Methyl-N-({5-[4-(1-methylethyl)-1-piperazinyl]imidazo[1,2-a]pyridin-2-yl}methyl)-5,6,7,8-tetrahydro-8-quinolinamine;
N-Methyl-N-{[5-(1-piperazinyl)imidazo[1,2-a]pyridin-2-yl]methyl}-5,6,7,8-tetrahydro-8-quinolinamine;
N,N,N′-Trimethyl-N′-(2-{[methyl(5,6,7,8-tetrahydro-8-quinolinyl)amino]methyl}imidazo[1,2-a]pyridin-5-yl)-1,2-ethanediamine;
N-(1-Methylethyl)-N-{[5-(4-methyl-1-piperazinyl)imidazo[1,2-a]pyridin-2-yl]methyl}-5,6,7,8-tetrahydro-8-quinolinamine; and
N-(1-Methylethyl)-N-({5-[4-(1-methylethyl)-1-piperazinyl]imidazo[1,2-a]pyridin-2-yl}methyl)-5,6,7,8-tetrahydro-8-quinolinamine.

Compounds of the present invention include:
(5-(4-Methyl-1-piperazinyl)-2-{[methyl(5,6,7,8-tetrahydro-8-quinolinyl)amino]methyl}imidazo[1,2-a]pyridin-3-yl)methanol;[2-({{(1S)-1-[4-(Methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({methyl[(8S)-5,6,7,8-tetrahydro-8-quinolinyl]amino}methyl)imidazo[1,2-a]pyridin-3-yl]methanol;
[5-[[2-(Dimethylamino)ethyl](methyl)amino]-2-({methyl[(8S)-5,6,7,8-tetrahydro-8-quinolinyl]amino}methyl)imidazo[1,2-a]pyridin-3-yl]methanol;

(5-(4-Methyl-1-piperazinyl)-2-{[methyl(6,7,8,9-tetrahydro-5H-cyclohepta[b]pyridin-9-yl)amino]methyl}imidazo[1,2-a]pyridin-3-yl)methanol;
[2-({Ethyl[(8S)-5,6,7,8-tetrahydro-8-quinolinyl]amino}methyl)-5-(4-methyl-1-piperazinyl)imidazo[1,2-a]pyridin-3-yl]methanol;
[2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({propyl[(8S)-5,6,7,8-tetrahydro-8-quinolinyl]amino}methyl)imidazo[1,2-a]pyridin-3-yl]methanol;
[2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({(phenylmethyl)[(8S)-5,6,7,8-tetrahydro-8-quinolinyl]amino}methyl)imidazo[1,2-a]pyridin-3-yl]methanol;
{5-(4-Methyl-1-piperazinyl)-2-[((8S)-5,6,7,8-tetrahydro-8-quinolinyl{[4-(trifluoromethyl)phenyl]methyl}amino)methyl]imidazo[1,2-a]pyridin-3-yl}methanol;
[5-(Hexahydropyrrolo[1,2-a]pyrazin-2(1H)-yl)-2-({methyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-(Hexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol;
[5-(4-Methylhexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol;
[5-[Methyl(1-methyl-3-pyrrolidinyl)amino]-2-({methyl[(8S)-5,6,7,8-tetrahydro-8-quinolinyl]amino}methyl)imidazo[1,2-a]pyridin-3-yl]methanol;
[5-(4-Ethyl-1-piperazinyl)-2-({methyl[(8S)-5,6,7,8-tetrahydro-8-quinolinyl]amino}methyl)imidazo[1,2-a]pyridin-3-yl]methanol;
[5-[4-(1-Methylethyl)-1-piperazinyl]-2-({methyl[(8S)-5,6,7,8-tetrahydro-8-quinolinyl]amino}methyl)imidazo[1,2-a]pyridin-3-yl]methanol;
[5-[(3S)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-Amino-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Methylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol;
{2-({(Cyclopropylmethyl)[(8S)-5,6,7,8-tetrahydro-8-quinolinyl]amino}methyl)-5-[(3R)-3-(dimethylamino)-1-pyrrolidinyl]imidazo[1,2-a]pyridin-3-yl}methanol;
1-[5-(4-Methyl-1-piperazinyl)-2-({methyl[(8S)-5,6,7,8-tetrahydro-8-quinolinyl]amino}methyl)imidazo[1,2-a]pyridin-3-yl]ethanol;
1-[5-(4-Methyl-1-piperazinyl)-2-({methyl[(8S)-5,6,7,8-tetrahydro-8-quinolinyl]amino}methyl)imidazo[1,2-a]pyridin-3-yl]-1-propanol;
(8S)—N-Methyl-N-{[3-[(methyloxy)methyl]-5-(4-methyl-1-piperazinyl)imidazo[1,2-a]pyridin-2-yl]methyl}-5,6,7,8-tetrahydro-8-quinolinamine;
(8S)—N-{[3-[(Ethyloxy)methyl]-5-(4-methyl-1-piperazinyl)imidazo[1,2-a]pyridin-2-yl]methyl}-N-methyl-5,6,7,8-tetrahydro-8-quinolinamine;
[5-(4-Methyl-1-piperazinyl)-2-(1-{methyl[(8S)-5,6,7,8-tetrahydro-8-quinolinyl]amino}ethyl)imidazo[1,2-a]pyridin-3-yl]methanol;
2,2,2-Trifluoro-1-(5-(4-methyl-1-piperazinyl)-2-{[methyl(5,6,7,8-tetrahydro-8-quinolinyl)amino]methyl}imidazo[1,2-a]pyridin-3-yl)ethanol; and pharmaceutically acceptable salts and esters thereof.

One aspect of the invention includes compounds of the following group:
[5-(4-Methyl-1-piperazinyl)-2-({methyl[(8S)-5,6,7,8-tetrahydro-8-quinolinyl]amino}methyl)imidazo[1,2-a]pyridin-3-yl]methanol;
(5-(4-Methyl-1-piperazinyl)-2-{[methyl(6,7,8,9-tetrahydro-5H-cyclohepta[b]pyridin-9-yl)amino]methyl}imidazo[1,2-a]pyridin-3-yl)methanol;
[2-({Ethyl[(8S)-5,6,7,8-tetrahydro-8-quinolinyl]amino}methyl)-5-(4-methyl-1-piperazinyl)imidazo[1,2-a]pyridin-3-yl]methanol;
[2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({propyl[(8S)-5,6,7,8-tetrahydro-8-quinolinyl]amino}methyl)imidazo[1,2-a]pyridin-3-yl]methanol;
[2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol;
[5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-(Hexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol;
[5-(4-Methylhexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol;
[5-[Methyl(1-methyl-3-pyrrolidinyl)amino]-2-({methyl[(8S)-5,6,7,8-tetrahydro-8-quinolinyl]amino}methyl)imidazo[1,2-a]pyridin-3-yl]methanol;
[5-(4-Ethyl-1-piperazinyl)-2-({methyl[(8S)-5,6,7,8-tetrahydro-8-quinolinyl]amino}methyl)imidazo[1,2-a]pyridin-3-yl]methanol;
[5-[4-(1-Methylethyl)-1-piperazinyl]-2-({methyl[(8S)-5,6,7,8-tetrahydro-8-quinolinyl]amino}methyl)imidazo[1,2-a]pyridin-3-yl]methanol;
[5-[(3S)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-Amino-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Methylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol;
{2-({(Cyclopropylmethyl)[(8S)-5,6,7,8-tetrahydro-8-quinolinyl]amino}methyl)-5-[(3R)-3-(dimethylamino)-1-pyrrolidinyl]imidazo[1,2-a]pyridin-3-yl}methanol;
[5-(4-Amino-1-piperidinyl)-2-({methyl[(8S)-5,6,7,8-tetrahydro-8-quinolinyl]amino}methyl)imidazo[1,2-a]pyridin-3-yl]methanol;
1-[5-(4-Methyl-1-piperazinyl)-2-({methyl[(8S)-5,6,7,8-tetrahydro-8-quinolinyl]amino}methyl)imidazo[1,2-a]pyridin-3-yl]ethanol;
1-[5-(4-Methyl-1-piperazinyl)-2-({methyl[(8S)-5,6,7,8-tetrahydro-8-quinolinyl]amino}methyl)imidazo[1,2-a]pyridin-3-yl]-1-propanol;
[5-(4-Methyl-1-piperazinyl)-2-(1-{methyl[(8S)-5,6,7,8-tetrahydro-8-quinolinyl]amino}ethyl)imidazo[1,2-a]pyridin-3-yl]methanol; and pharmaceutically acceptable salts and esters thereof.

One aspect of the invention includes compounds:
(5-(4-Methyl-1-piperazinyl)-2-{[methyl(5,6,7,8-tetrahydro-8-quinolinyl)amino]methyl}imidazo[1,2-a]pyridin-3-yl)methanol;
[5-(4-Methyl-1-piperazinyl)-2-({methyl[(8S)-5,6,7,8-tetrahydro-8-quinolinyl]amino}methyl)imidazo[1,2-a]pyridin-3-yl]methanol;
[2-({Ethyl[(8S)-5,6,7,8-tetrahydro-8-quinolinyl]amino}methyl)-5-(4-methyl-1-piperazinyl)imidazo[1,2-a]pyridin-3-yl]methanol;
[2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({propyl[(8S)-5,6,7,8-tetrahydro-8-quinolinyl]amino}methyl)imidazo[1,2-a]pyridin-3-yl]methanol;
[2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Ethyl-1-piperazinyl)-2-({methyl[(8S)-5,6,7,8-tetrahydro-8-quinolinyl]amino}methyl)imidazo[1,2-a]pyridin-3-yl]methanol;
[5-[4-(1-Methylethyl)-1-piperazinyl]-2-({methyl[(8S)-5,6,7,8-tetrahydro-8-quinolinyl]amino}methyl)imidazo[1,2-a]pyridin-3-yl]methanol; and pharmaceutically acceptable salts or esters thereof.

Compounds of the present invention also include:
[5-(4-Methyl-1-piperazinyl)-2-({methyl[(8S)-5,6,7,8-tetrahydro-8-quinolinyl]amino}methyl)imidazo[1,2-a]pyridin-3-yl]methanol;
[2-({Ethyl[(8S)-5,6,7,8-tetrahydro-8-quinolinyl]amino}methyl)-5-(4-methyl-1-piperazinyl)imidazo[1,2-a]pyridin-3-yl]methanol;
[2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({propyl[(8S)-5,6,7,8-tetrahydro-8-quinolinyl]amino}methyl)imidazo[1,2-a]pyridin-3-yl]methanol;
[2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol; and pharmaceutically acceptable salts or esters thereof.

Compounds of the present invention also include:
[5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[Methyl(1-methyl-3-pyrrolidinyl)amino]-2-({methyl[(8S)-5,6,7,8-tetrahydro-8-quinolinyl]amino}methyl)imidazo[1,2-a]pyridin-3-yl]methanol;
[5-[(3S)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-Amino-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Methylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol;
{2-({(Cyclopropylmethyl)[(8S)-5,6,7,8-tetrahydro-8-quinolinyl]amino}methyl)-5-[(3R)-3-(dimethylamino)-1-pyrrolidinyl]imidazo[1,2-a]pyridin-3-yl}methanol;
[5-(4-Amino-1-piperidinyl)-2-({methyl[(8S)-5,6,7,8-tetrahydro-8-quinolinyl]amino}methyl)imidazo[1,2-a]pyridin-3-yl]methanol; and pharmaceutically acceptable salts or esters thereof.

One aspect of the present invention includes the compounds substantially as hereinbefore defined with reference to any one of the Examples.

One aspect of the present invention includes a pharmaceutical composition comprising one or more compounds of the present invention and a pharmaceutically acceptable carrier.

One aspect of the present invention includes one or more compounds of the present invention for use as an active therapeutic substance.

One aspect of the present invention includes one or more compounds of the present invention for use in the treatment or prophylaxis of diseases and conditions caused by inappropriate activity of CXCR4.

One aspect of the present invention includes one or more compounds of the present invention for use in the treatment or prophylaxis of diseases and conditions caused by inappropriate activity of CCR5.

One aspect of the present invention includes one or more compounds of the present invention for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scieroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. Preferably the condition or disease is HIV infection rheumatoid arthritis, inflammation, or cancer.

One aspect of the present invention includes the use of one or more compounds of the present invention in the manufacture of a medicament for use in the treatment or prophylaxis of a condition or disease modulated by a chemokine receptor. Preferably the chemokine receptor is CXCR4 or CCR5.

One aspect of the present invention includes use of one or more compounds of the present invention in the manufacture of a medicament for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers. Preferably the use relates to a medicament wherein the condition or disorder is HIV infection rheumatoid arthritis, inflammation, or cancer.

One aspect of the present invention includes a method for the treatment or prophylaxis of a condition or disease modulated by a chemokine receptor comprising the administration of one or more compounds of the present invention. Preferably the chemokine receptor is CXCR4 or CCR5.

One aspect of the present invention includes a method for the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus; spondylo-arthropathies, scleroderma; psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of one or more compounds of the present invention.

One aspect of the present invention includes a method for the treatment or prophylaxis of HIV infection rheumatoid arthritis, inflammation, or cancer comprising the administration of one or more compounds of the present invention.

### DETAILED DESCRIPTION OF THE INVENTION

Terms are used within their accepted meanings. The following definitions are meant to clarify, but not limit, the terms defined.

As used herein the term “alkyl” refers to a straight or branched chain hydrocarbon, preferably having from one to twelve carbon atoms. Examples of “alkyl” as used herein include, but are not limited to, methyl, ethyl, propyl, isopropyl, isobutyl, n-butyl, tert-butyl, isopentyl, n-pentyl.

As used throughout this specification, the preferred number of atoms, such as carbon atoms, will be represented by, for example, the phrase “Cₓ—C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds. Examples include, but are not limited to, vinyl, allyl, and the like.

As used herein the term “alkynyl” refers to a straight or branched chain aliphatic hydrocarbon containing one or more carbon-to-carbon triple bonds. Examples include, but are not limited to, ethynyl and the like.

As used herein, the term “alkylene” refers to an optionally substituted straight or branched chain divalent hydrocarbon radical, preferably having from one to ten carbon atoms. Examples of “alkylene” as used herein include, but are not limited to, methylene, ethylene, n-propylene, n-butylene, and the like. Preferred substituents include C₁-C₆ alkyl, oxo and hydroxyl.

As used herein, the term “alkenylene” refers to a straight or branched chain divalent hydrocarbon radical, preferably having from one to ten carbon atoms, containing one or more carbon-to-carbon double bonds. Examples include, but are not limited to, vinylene, allylene or 2-propenylene, and the like.

As used herein, the term “alkynylene” refers to a straight or branched chain divalent hydrocarbon radical, preferably having from one to ten carbon atoms, containing one or more carbon-to-carbon triple bonds. Examples include, but are not limited to, ethynylene and the like.

As used herein, the term “cycloalkyl” refers to an optionally substituted non-aromatic cyclic hydrocarbon ring. Exemplary “cycloalkyl” groups include, but are not limited to, cyclopropyl, cyclobutyl, cyclopentyl, cyclohexyl, and cycloheptyl. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, and cycloheptenyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, and cycloheptylene. Preferred substituents include C₁-C₆ alkyl, oxo and hydroxyl.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, and cycloheptenylene.

As used herein, the term “heterocycle” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. More preferably, the heteroatom is N.

Preferably the heterocyclyl ring is three to twelve-membered and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, piperazine, pyrrolidine, morpholine, tetrahydrothiopyran, aziridine, azetidine and tetrahydrothiophene. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “aryl” refers to an optionally substituted benzene ring or to an optionally substituted fused benzene ring system, for example anthracene, phenanthrene, or naphthalene ring systems. Examples of “aryl” groups include, but are not limited to, phenyl, 2-naphthyl, and 1-naphthyl. Preferred substituent groups include alkyl, alkenyl, alkynyl, alkoxy, hydroxyl, halogen, haloalkyl, cycloalkyl, cycloalkoxy, cyano, amide, amino, and alkylamino.

As used herein, the term “heteroaryl” refers to an optionally substituted monocyclic five to seven membered aromatic ring, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. Preferably, the heteroatom is N.

Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidizolyl, imidazopyridinyl, pyrazolopyridinyl, and pyrazolopyrimidinyl. Preferred substituent groups include alkyl, alkenyl, alkynyl, alkoxy, hydroxyl, halogen, haloalkyl, cycloalkyl, cycloalkoxy, cyano, amide, amino, and alkylamino. As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “alkoxy” refers to a group —OR¹⁰, where R′ is alkyl as defined.

As used herein the term “cycloalkoxy” refers to a group —OR¹⁰, where R′ is cycloalkyl as defined.

As used herein the term “alkoxycarbonyl” refers to groups such as: 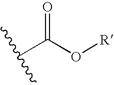
where the R′ represents an alkyl group as herein defined.

As used herein the term “aryloxycarbonyl” refers to groups such as: 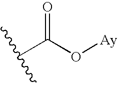
where the Ay represents an aryl group as herein defined.

As used herein the term “nitro” refers to a group —NO₂.

As used herein the term “cyano” refers to a group —CN.

As used herein the term “azido” refers to a group —N₃.

As used herein the term amino refers to a group —NR′R″, where R′ and R″ independently represent H, alkyl, alkenyl, alkynyl, cycloalkyl, heterocyclyl, aryl, or heteroaryl. Similarly, the term “alkylamino” includes an alkylene linker through which the amino group is attached. Examples of “alkylamino” as used herein include groups such as —(CH₂)ₓNH₂, where x is preferably 1 to 6.

As used herein the term “amide” refers to a group —C(O)NR′R″, where R′ and R″ independently represent H, alkyl, alkenyl, alkynyl, cycloalkyl, heterocyclyl, aryl, or heteroaryl. Examples of “amide” as used herein include groups such as —C(O)NH₂, —C(O)NH(CH₃), —C(O)N(CH₃)₂, and the like.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted specifically. Rather, those of ordinary skill in the art will appreciate that the phrase is included to provide for obvious modifications, which are encompassed within the scope of the appended claims.

The compounds of formulas (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically and/or diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “modulators” as used herein is intended to encompass antagonist, agonist, inverse agonist, partial agonist or partial antagonist, inhibitors and activators. In one preferred embodiment of the present invention, the compounds demonstrate protective effects against HIV infection by inhibiting binding of HIV to a chemokine receptor such as CXCR4 and/or CCR5 of a target cell. The invention includes a method that comprises contacting the target cell with an amount of the compound that is effective at inhibiting the binding of the virus to the chemokine receptor.

In addition to the role chemokine receptors play in HIV infection this receptor class has also been implicated in a wide variety of diseases. Thus CXCR4 modulators may also have a therapeutic role in the treatment of diseases associated with hematopoiesis, including but not limited to, controlling the side effects of chemotherapy, enhancing the success of bone marrow transplantation, enhancing wound healing and burn treatment, as well as combating bacterial infections in leukemia. In addition, compounds may also have a therapeutic role in diseases associated with inflammation, including but not limited to inflammatory or allergic diseases such as asthma, allergic rhinitis, hypersensitivity lung diseases, hypersensitivity pneumonitis, eosinophilic pneumonitis, delayed-type hypersensitivity, interstitial lung disease (ILD) (e.g. idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies, insect sting allergies; autoimmune diseases such as rheumatoid arthritis, psoriatic arthritis, systemic lupus erythematosus, myastenia gravis, juvenile onset diabetes; glomerulonephritis, autoimmune throiditis, graft rejection, including allograft rejection or graft-versus-host disease; inflammatory bowel diseases, such as Crohn's disease and ulcerative colitus; spondyloarthropathies; scleroderma; psoriasis (including T-cell-mediated psoriasis) and inflammatory dermatoses such as dermatitis, eczema, atopic dermatitis, allergic contact dermatitis, urticaria, vasculitis (e.g. necrotizing, cutaneous, and hypersensitivity vasculitis); eoosinophilic myotis, eosinophilic fasciitis; and cancers.

For use in therapy, therapeutically effective amounts of a compound of formula (I), as well as salts, solvates, and physiological functional derivatives thereof, may be administered as the raw chemical. Additionally, the active ingredient may be presented as a pharmaceutical composition.

Accordingly, the invention further provides pharmaceutical compositions that include effective amounts of compounds of the formula (I) and salts, solvates, and physiological functional derivatives thereof, and one or more pharmaceutically acceptable carriers, diluents, or excipients. The compounds of formula (I) and salts, solvates, and physiologically functional derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formula (I) or salts, solvates, and physiological functional derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of formula (I) for the treatment of humans suffering from frailty, generally, should be in the range of 0.1 to 100 mg/kg body weight of recipient (mammal) per day. More usually the effective amount should be in the range of 0.1 to 10 mg/kg body weight per day. Thus, for a 70 kg adult mammal one example of an actual amount per day would usually be from 7 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of formula (I) per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) and salts, solvates, and physiological functional derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of formula (I) and salts, solvates, and physiologically functional derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, and physiologically functional derivatives thereof, may be employed alone or in combination with other therapeutic agents. The compound(s) of formula (I) and the other pharmaceutically active agent(s) may be administered together or separately and, when administered separately, administration may occur simultaneously or sequentially, in any order. The amounts of the compound(s) of formula (I) and the other pharmaceutically active agent(s) and the relative timings of administration will be selected in order to achieve the desired combined therapeutic effect. The administration in combination of a compound of formula (I) salts, solvates, or physiologically functional derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions. The compounds may be used in combination with any other pharmaceutical composition where such combined therapy may be useful to modulate chemokine receptor activity and thereby prevent and treat inflammatory and/or immunoregulatory diseases.

The present invention may be used in combination with one or more agents useful in the prevention or treatment of HIV. Examples of such agents include:

Nucleotide reverse transcriptase inhibitors such as zidovudine, didanosine, lamivudine, zalcitabine, abacavir, stavidine, adefovir, adefovir dipivoxil, fozivudine, todoxil, and similar agents;

Non-nucleotide reverse transcriptase inhibitors (including an agent having anti-oxidation activity such as immunocal, oltipraz, etc.) such as nevirapine, delavirdine, efavirenz, loviride, immunocal, oltipraz, and similar agents;

Protease inhibitors such as saquinavir, ritonavir, indinavir, nelfinavir, aprenavir, palinavir, lasinavir, and similar agents;

Entry inhibitors such as T-20, T-1249, PRO-542, PRO-140, TNX-355, BMS-806, 5-Helix and similar agents;

Integrase inhibitors such as L-870, 180 and similar agents;

Budding inhibitors such as PA-344 and PA-457, and similar agents; and

Other CXCR4 and/or CCR5 inhibitors such as Sch-C, Sch-D, TAK779, UK 427,857, TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

The scope of combinations of compounds of this invention with HIV agents is not limited to those mentioned above, but includes in principle any combination with any pharmaceutical composition useful for the treatment of HIV. As noted, in such combinations the compounds of the present invention and other HIV agents may be administered separately or in conjunction. In addition, one agent may be prior to, concurrent to, or subsequent to the administration of other agent(s).

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of formula (I).

Those skilled in the art will recognize if a stereocenter exists in compounds of formula (I). Accordingly, the scope of the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### EXPERIMENTAL SECTION

Abbreviations:

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| Hz (Hertz); | MHz (megahertz); |
| mol (moles); | mmol (millimoles); |
| RT (room temperature); | h (hours); |
| min (minutes); | TLC (thin layer chromatography); |
| mp (melting point); | RP (reverse phase); |
| Tᵣ (retention time); | TFA (trifluoroacetic acid); |
| TEA (triethylamine); | THF (tetrahydrofuran); |
| TFAA (trifluoroacetic anhydride); | CD₃OD (deuterated methanol); |
| CDCl₃ (deuterated chloroform); | DMSO (dimethylsulfoxide); |
| SiO₂ (silica); | atm (atmosphere); |
| EtOAc (ethyl acetate); | CHCl₃ (chloroform); |
| HCl (hydrochloric acid); | Ac (acetyl); |
| DMF (N,N-dimethylformamide); | Me (methyl); |
| Cs₂CO₃ (cesium carbonate); | EtOH (ethanol); |
| Et (ethyl); | tBu (tert-butyl); |
| MeOH (methanol) | p-TsOH (p-toluenesulfonic acid); |
| MP-TsOH (polystyrene resin bound | |
| equivalent of p-TsOH from Argonaut | |
| Technologies). | |

Unless otherwise indicated, all temperatures are expressed in OC (degrees Centigrade). All reactions conducted at room temperature unless otherwise noted.

¹H-NMR spectra were recorded on a Varian VXR-300, a Varian Unity-300, a Varian Unity-400 instrument, or a General Electric QE-300. Chemical shifts are expressed in parts per million (ppm, 6 units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds can be assigned by Ab Initio Vibrational Circular Dichroism (VCD) Spectroscopy. The experimental VCD spectra were acquired in CDCl₃ using a Bomem Chiral® VCD spectrometer operating between 2000 and 800 cm⁻¹. The Gaussian 98 Suite of computational programs was used to calculate model VCD spectrums. The stereochemical assignments were made by comparing this experimental spectrum to the VCD spectrum calculated for a model structure with (R)- or (S)-configuration. Incorporated by reference with regard to such spectroscopy are: J. R. Chesseman, M. J. Frisch, F. J. Devlin and P. J. Stephens, Chem. Phys. Lett. 252 (1996) 211; P. J. Stephens and F. J. Devlin, Chirality 12 (2000) 172; and Gaussian 98, Revision A. 11.4, M. J. Frisch et al., Gaussian, Inc., Pittsburgh Pa., 2002.

Compounds of formula (I) where R is H, t is 1 and all other variables are as defined herein can be prepared according to Scheme 1:

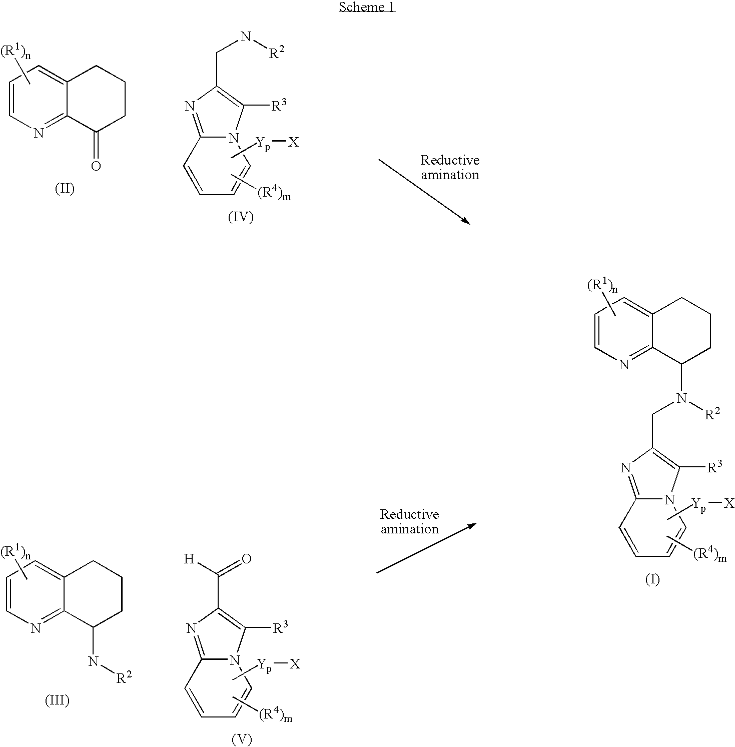

Compounds of formula (I-G) can be prepared in a similar method wherein R³ in formulas (IV), (V) and (I) is R^{a}OR⁵. More specifically, compounds of formula (I) can be prepared by reacting a compound of formula (II) with a compound (IV) or alternatively reacting a compound of formula (III) with a compound of formula (V) under reductive conditions. The reductive amination can be carried out by treating the compound of formula (II) or (III) with a compound of formula (IV) or (V) in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

Compounds of formula (II) can be prepared as described in the literature (J. Org. Chem., 2002, 67, 2197-2205, herein incorporated by reference with regard to such synthesis). Compounds of formula (III) can be prepared by reductive amination of compounds of formula (II) using processes well known to those skilled in the art of organic synthesis. Compounds of formula (V) can be prepared by methods similar to those described in the literature (J. Heterocyclic Chemistry, 1992, 29, 691-697, incorporated by reference with regard to such synthesis). Compounds of formula (IV) can be prepared from compounds of formula (V) via reductive amination using processes known to those skilled in the art.

As is evident to one skilled in the art a compound where R is not H can be prepared in a similar fashion as outlined in Scheme 1. Also evident to one skilled in the art is that compounds of formula I where t is 0 or t is 2 can be prepared in a similar fashion as outlined in Scheme 1.

Compounds of formula (I) wherein R is H, t is 1, LV is a suitable leaving group (e.g., halogen, mesylate, tosylate, or the like) and all other variables are as defined in connection with formula (I) can be prepared according to Scheme 2:

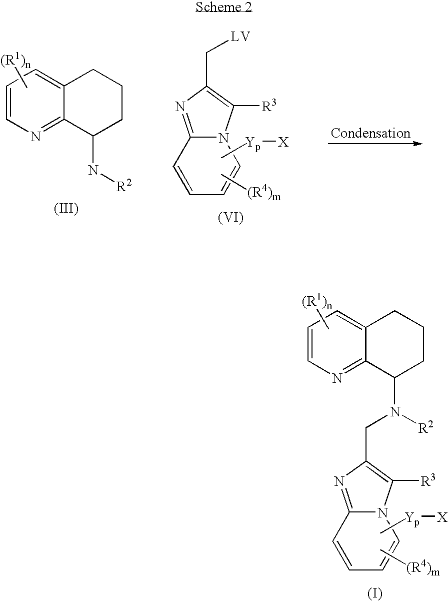

Compounds of formula (I-G) can be prepared in a similar method wherein R³ in formulas (VI) and (I) is R^{a}OR⁵. Compound of formula (I) can be prepared by reacting a compound of formula (III) with a compound of formula (VI) where LV is a leaving group (e.g., halogen, mesylate, tosylate, or the like). This condensation is typically carried out in a suitable solvent optionally in the presence of base, optionally with heating. Suitable solvents include tetrahydrofuran, dioxane, acetonitrile, nitromethane, N,N-dimethylformamide, and the like. Suitable bases include triethylamine, pyridine, dimethylaminopyridine, N,N-diisopropylethylamine, potassium carbonate, sodium carbonate, cesium carbonate and the like. The reaction can be carried out at room temperature or optionally heated to 30-200° C. Optionally the reaction can be carried out in a microwave. A catalyst, such as potassium iodide, tertbutylammonium iodide, or the like, can optionally be added to the reaction mixture. Compounds of formula (VI) can be prepared by methods similar to those described in the literature (Chem. Pharm. Bull. 2000, 48, 935; Tetrahedron, 1991, 47, 5173; Tetrahedron Lett. 1990, 31, 3013; J. Heterocyclic Chemistry, 1988, 25, 129; Chemistry of Heterocyclic Compounds, 2002, 38, 590; each incorporated by reference with regard to such synthesis).

Compounds of formula (I-A) (i.e. formula (I) wherein R is H, t is 1 and all other variables are as defined with respect to formula (I)) can be prepared according to

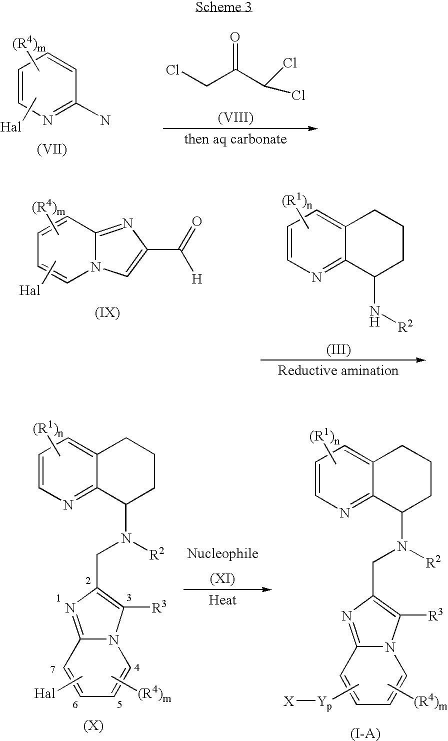

More specifically, compounds of formula (I-A) can be prepared by treating a compound of formula (X) with a nucleophile. The reaction can be carried out by treating the compound of formula (X) with a suitable nucleophile, neat, or optionally in the presence of an inert solvent. The reaction may be heated to 50-200° C. or performed at ambient temperature. Optionally the reaction may be carried out in a microwave. Compounds of formula (X) can be prepared from a compound of formula (IX) and a compound of formula (III) by reductive amination. Aldehydes of formula (IX) can be prepared by methods similar to those described in the literature (e.g. J. Heterocyclic Chemistry, 1992, 29, 691-697, incorporated by reference with regard to such synthesis).

Alternatively, compounds of formula (I-B) (i.e. formula (I) wherein R is H, t is 1, p is 0 and X is -Het and all other variables are as defined with respect to formula (I)) can be prepared according to Scheme 4:

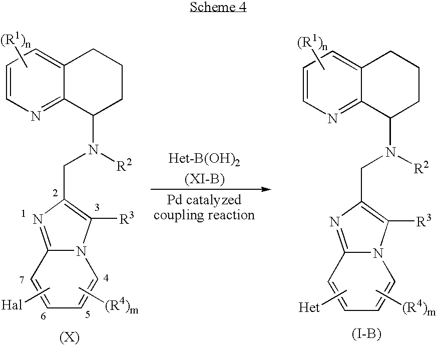

For preparation of compounds of formula (I-G), R³ is R^{a}OR⁵ in formulas (X) and (I-B). As illustrated in Scheme 4, a compound of formula (X) can be converted to a compound of formula (I-B) via a coupling of compound of formula (X) and a compound of formula (XI-B). The coupling reaction depicted below is a Suzuki coupling, other coupling reactions (e.g. Stille) well known to those skilled in the art of organic chemistry can also be used to make compounds of formula (I-B). These coupling reactions are well known to those skilled in the art of organic synthesis.

A compound of formula (I-C) (i.e. a compound of formula (I) wherein R is H, t is 1, p is 0, X is AyR^{a}N(R¹⁰)₂ and all other variables are as defined with respect to formula (I)) can be prepared according to Scheme 5:

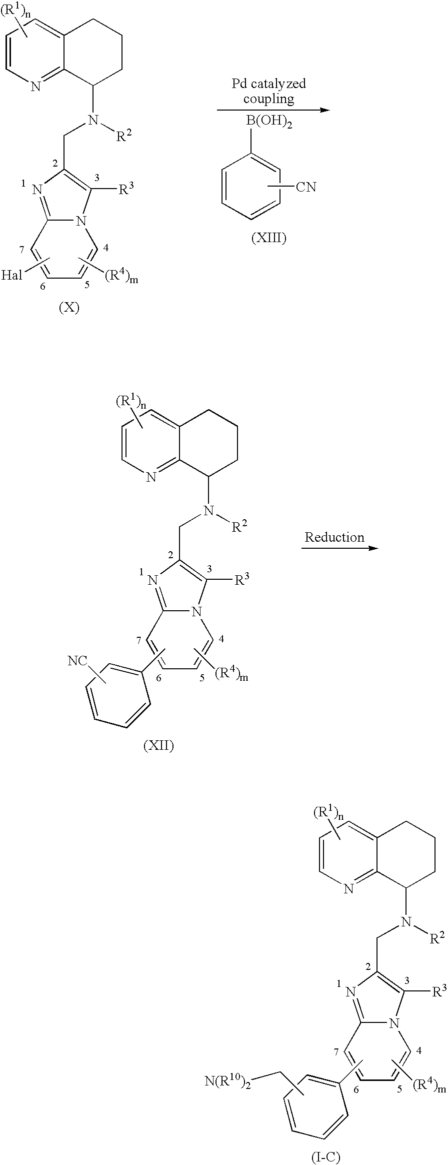

For preparation of compounds of formula (I-G), R³ is R^{a}OR⁵ in formulas (X), (XII) and (I-C). Optionally, as illustrated in Scheme 5, a compound of formula (X) can be coupled with a compound of formula (XIII) to form a compound of formula (XII). Reduction of compound of formula (XII) would give a compound of formula (I-C).

A compound of formula (I-D) (i.e. a compound of formula (I) wherein R is H, t is 1 and all other variables are as defined in connection with formula (I)) can be prepared according to Scheme 6.

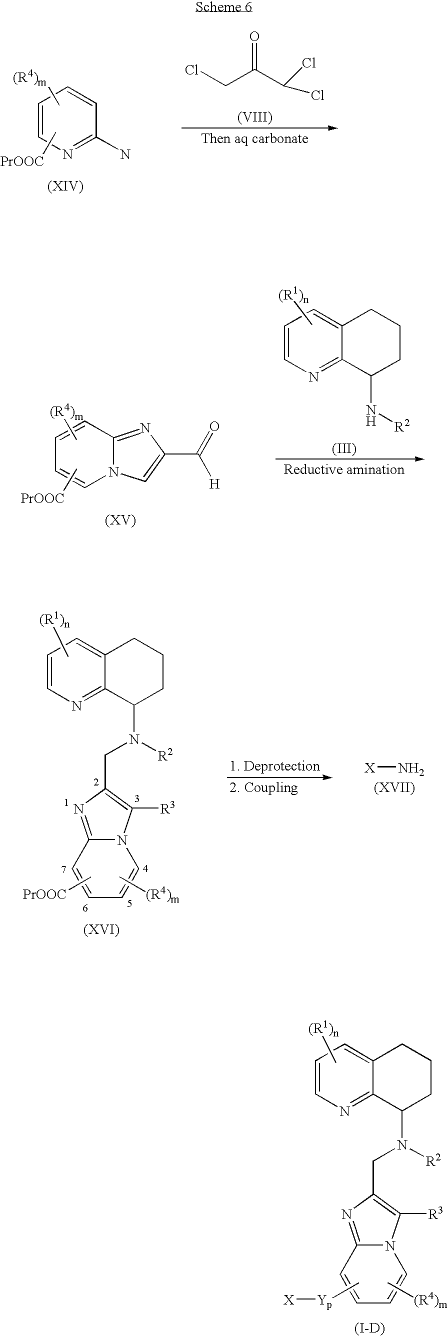

A compound of formula (I-D) (i.e. a compound of formula (I) where p is 1, Y is —C(O)NH—) where Pr is a suitable protecting group for a carboxylic acid, could optionally be formed from a compound of formula (XIV). A compound of formula (XVI) is deprotected, followed by coupling of the resulting acid with an amine compound of formula (XVII). This coupling can be carried out using a variety of coupling reagent well know to those skilled in the art of organic synthesis (e.g., EDC, HOBt/HBTu; BOPCI). The reaction can be carried out with heating or at ambient temperature. Suitable solvents for this reaction include acetonitrile, tetrahydrofuran, and the like.

Compounds of formula (I-G) wherein R is H and R^{a}OR⁵ is —CH₂OH, t is 1 and all other variables are as defined in connection with formula (I-G) can be prepared according to Scheme 7:

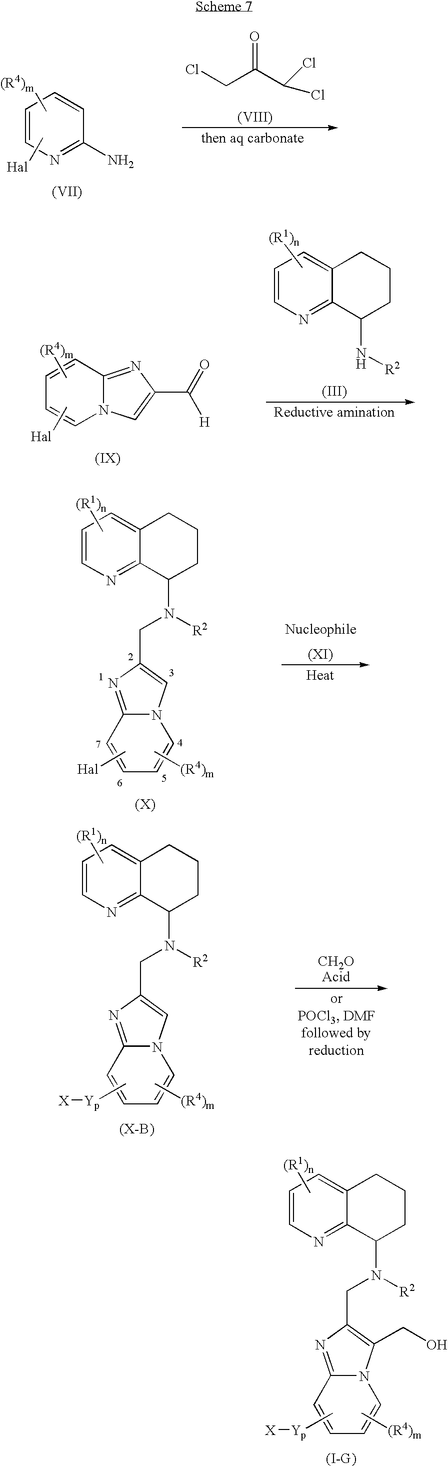

More specifically, compounds of formula (X-B) can be prepared by treating a compound of formula (X) with a nucleophile. The reaction can be carried out by treating the compound of formula (X) with a suitable nucleophile, neat, or optionally in the presence of an inert solvent. The reaction may be heated to 50-200° C. or performed at ambient temperature. Optionally the reaction may be carried out in a microwave. Compounds of formula (X) can be prepared from a compound of formula (IX) and a compound of formula (III) by reductive amination. Aldehydes of formula (IX) can be prepared by methods similar to those described in the literature (e.g. J. Heterocyclic Chemistry, 1992, 29, 691-697, incorporated by reference with regard to such synthesis).

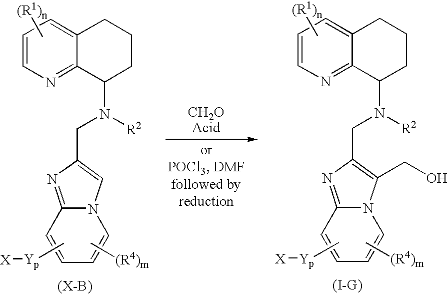

Compound of formula (I-G) can be prepared from compound of formula (X-B) via hydroxymethylation. Thus compound of formula (X-B) can be treated with formaldehyde or a suitable compound that generates formaldehyde in a suitable solvent optionally in the presence of an acid. Optionally the reaction can be heated between 30-150° C. Suitable solvents include water, acetic acid and the like. Suitable acids include acetic acid and the like.

Alternatively a compound of formula (I-G) can be prepared from a compound of formula (X-B) by a two step sequence. This involves treatment of compound of formula (X-B) with POCl₃ in N,N-dimethylformamide (formylation), followed by reduction of the aldehyde to an alcohol of formula (I-G). The reduction can be carried out by using any suitable reducing agent in a suitable solvent. An example of a suitable reducing agent include sodium borohydride, lithium borohydride, borane and the like. Suitable solvents include alcohols (methyl alcohol, ethyl alcohol) and the like.

Compounds of formula (I-G) wherein R is H, R^{a}OR⁵ is —CH₂OH, t is 1, p is 1, Y is C(O)NH and all other variables are as defined in connection with formula (I-G) can be prepared according to Scheme 8:

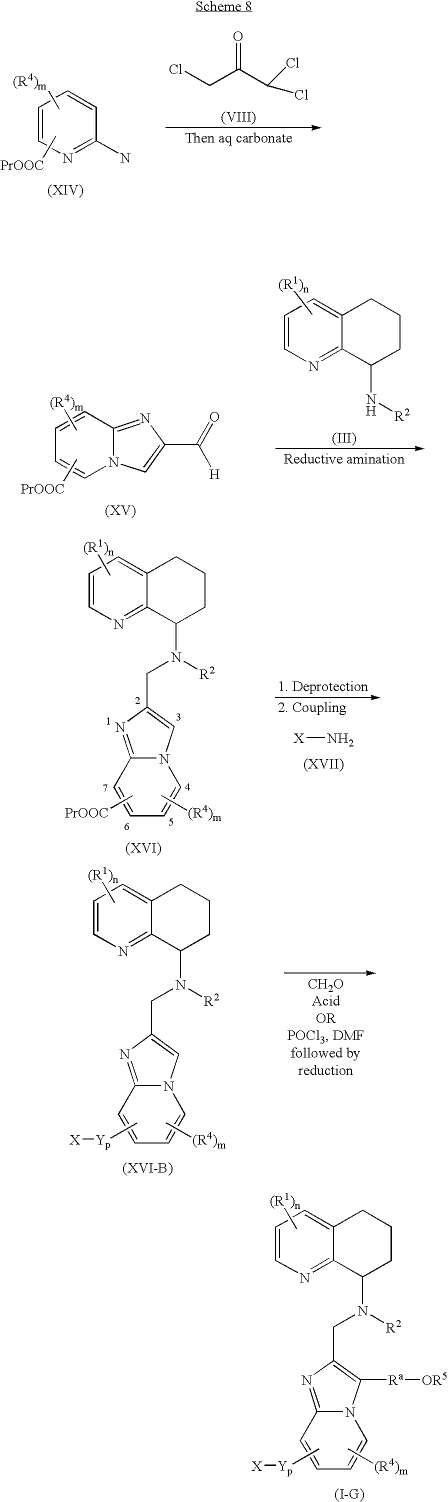

A compound of formula (XVI-B) where R is H, t is 1, p is 1 and Y is —C(O)NH— and Pr is a suitable protecting group for a carboxylic acid, could optionally be formed from a compound of formula (XIV). A compound of formula (XVI) is deprotected, followed by coupling of the resulting acid with an amine compound of formula (XVII). This coupling can be carried out using a variety of coupling reagent well know to those skilled in the art of organic synthesis (e.g., EDC, HOBt/HBTu; BOPCI). The reaction can be carried out with heating or at ambient temperature. Suitable solvents for this reaction include acetonitrile, tetrahydrofuran, and the like. Compound of formula (I-G) can be formed from a compound of formula (XVI-B) by hydroxymethylation as outlined in connection with previous Schemes.

A compound of formula (I-E) where R is H, R^{a}OR⁵ is CH₂OH, t is 1, p is 1 and Y is —NHC(O)— and all other variables are defined in connection with formula (I-G) could optionally be formed from a compound of formula (XVII) as outlined in Scheme 9:

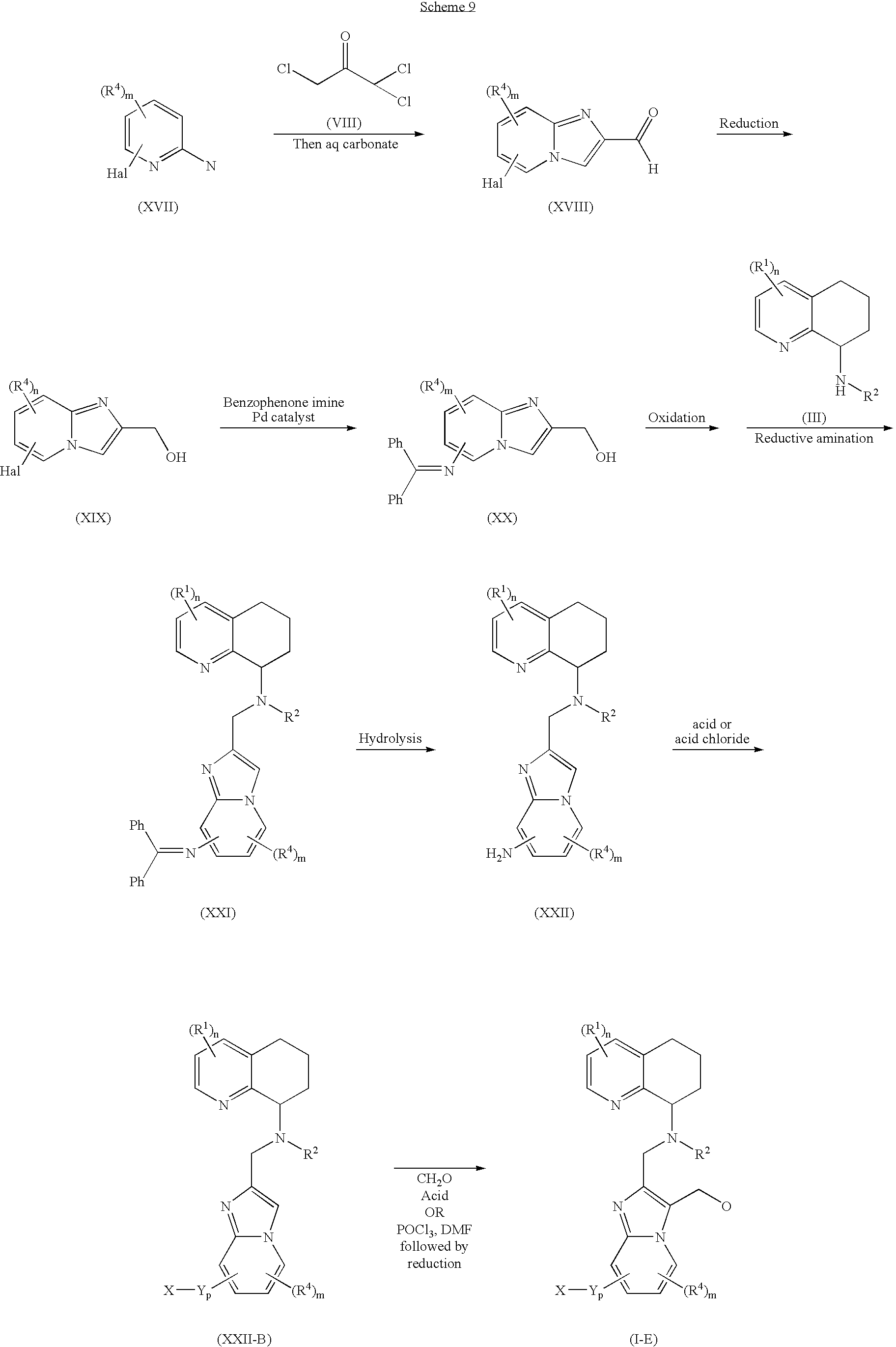

In more detail a compound of formula (XVIII) is reduced, followed by Pd catalyzed coupling with benzophenone imine to give a compound of formula (XX). This coupling can be carried out using a variety of palladium reagents and ligands well know to those skilled in the art of organic synthesis (e.g., Pd(OAc)₂ and BINAP). The reaction can be carried out with heating or at ambient temperature. Suitable solvents for this reaction include toluene, acetonitrile, tetrahydrofuran, and the like. Compound of formula (XX) can be oxidized to an aldehyde using any suitable oxidation method (e.g. MnO₂ in dichloromethane and the like) followed by reductive amination with compound of formula (III) to give a compound of formula (XXI). The reductive amination can be carried out by treating the compound of formula (III) with the aldehyde in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like. Hydrolysis of the benzophenone imine yields a compound of formula (XXII). Suitable hydrolysis conditions include treatment of compound of formula (XXI) with hydrochloric acid and the like in a suitable solvent, such as tetrahydrofuran. Treatment of an amine compound of formula (XXII) with an acid chloride or alternatively with an acid in the presence of a suitable coupling agent (e.g., EDC, HOBt/HBTu; BOPCI) gives a compound of formula (XXII-B). Conditions for coupling of an amine compound of formula (XXII) and an acid or an acid chloride are well know to those skilled in the art of organic synthesis. Compound of formula (I-E) can be formed from compound of formula (XXII-B) using hydroxymethylation conditions outlined in connections with previous Schemes.

A compound of formula (I-F) where R^{a}OR⁵ is CH₂OH, n and m are 0, t is 1, p is 0 and X is a piperazine suitably substituted with Z, where Z is C₁-C₆alkyl or C₃-C₈cycloalkyl and all other variables are as defined in connection with compound of formula (I-G) can be synthesized in a chiral fashion as outlined in Scheme 10:

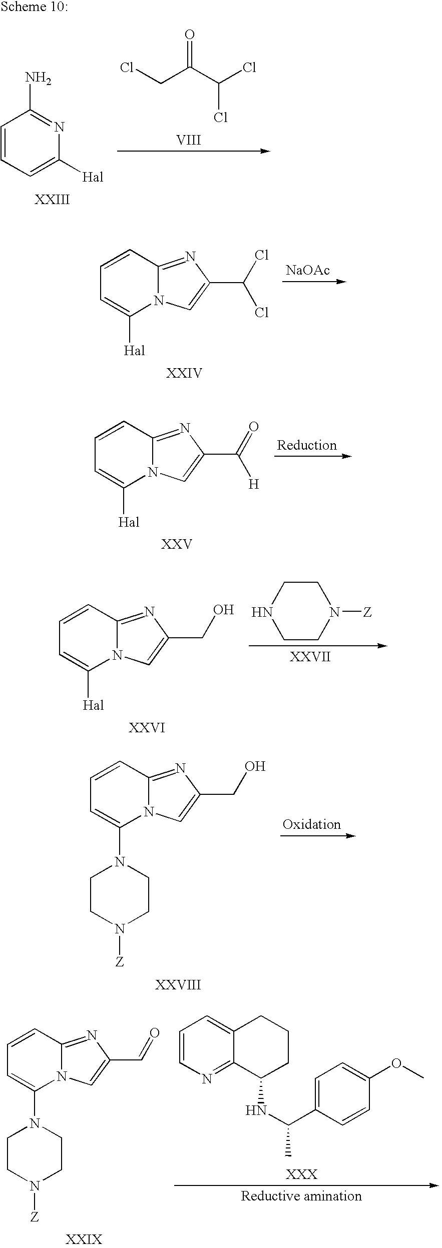

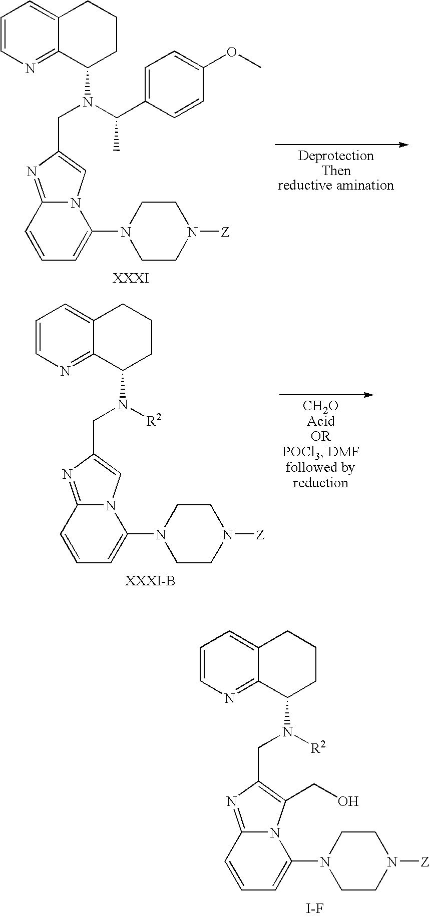

A compound of formula (I-F) can be prepared from compound of formula (XXXI-B).

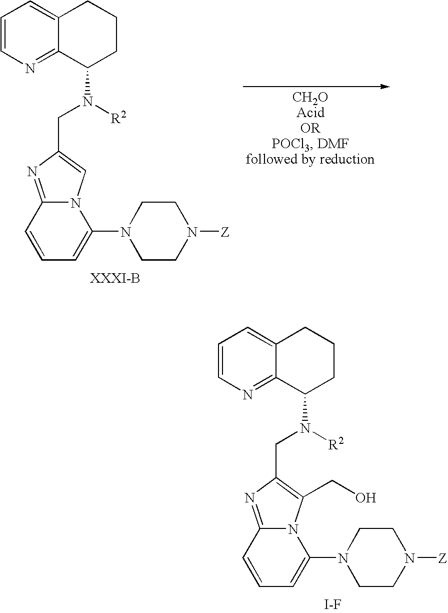

Compound of formula (I-F) can be prepared from compound of formula (XXXI-B) via hydroxymethylation. Thus compound of formula (XXXI-B) can be treated with formaldehyde or a suitable compound that generates formaldehyde in a suitable solvent optionally in the presence of an acid. Optionally the reaction can be heated between 30-150° C. Suitable solvents include water acetic acid and the like. Suitable acids include acetic acid and the like.

Alternatively a compound of formula (I-F) can be prepared from a compound of formula (XXXI-B) by a two step sequence. This involves treatment of compound of formula (XXXI-B) with POCl₃ in N,N-dimethylformamide (formylation), followed by reduction of the aldehyde to an alcohol of formula (I-F). The reduction can be carried out by using any suitable reducing agent in a suitable solvent. An example of a suitable reducing agent include sodium borohydride, lithium borohydride, borane and the like. Suitable solvents include alcohols (methyl alcohol, ethyl alcohol) and the like.

A compound of formula (XXXI-B) can be prepared from a compound of formula (XXXI)

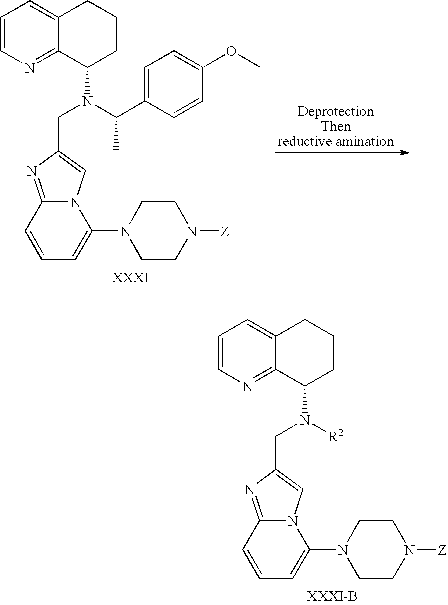

Treatment of compound of formula (XXXI) with a strong acid in a suitable solvent is an appropriate deprotection method. Suitable acids include trifluoroacetic acid and the like. Suitable solvents include dichloromethane, dichloroethane and the like. The reaction can optionally be heated. Alternative deprotection methods include use of Lewis acids (e.g. BCl₃, AlCl₃, BBr₃ and the like) or removal of the protecting group under reductive conditions (e.g. Pd on charcoal or PtO₂ under H₂ atmosphere). The resulting amine (compound of formula I where R² is H) can then be treated with a suitable aldehyde under reductive amination conditions to give a compound of formula (XXXI-B). The reductive amination can be carried out by treating the amine with the aldehyde in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like.

A compound of formula (XXXI) can be prepared from a compound of formula (XXIX) and compound of formula (XXX):

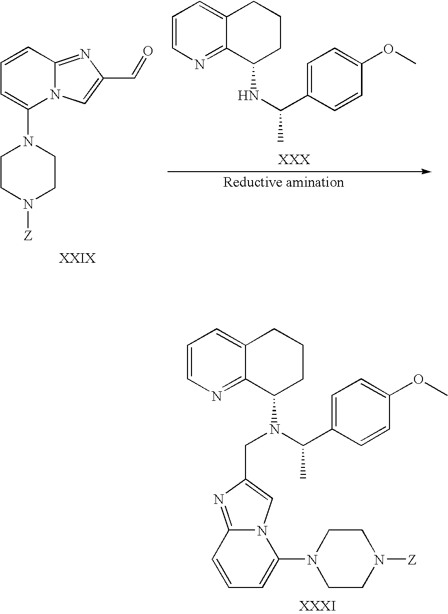

Reductive amination of compound of formula (XXIX) with a compound of formula (XXX) gives compounds of formula (XXXI). The reductive amination can be carried out in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like. Compound of formula (XXX) can be prepared form (S)-(−)-1-(4-methoxyphenyl)ethylamine and 6,7-dihydro-8(5H)-quinolinone (J. Org. Chem., 2002, 67, 2197-2205) by reductive amination.

A compound of formula (XXIX) can be prepared from a compound of formula (XXVIII).

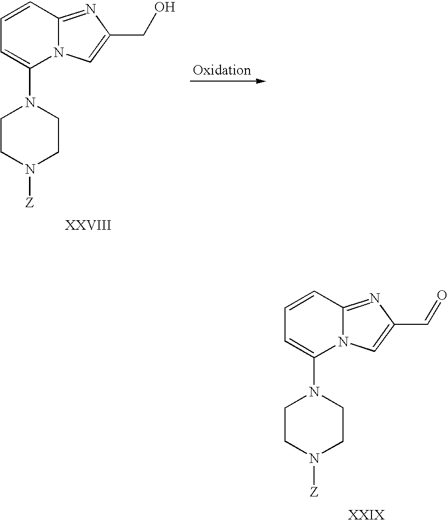

Oxidation of compound of formula (XXVIII) gives a compound of formula (XXIX). A suitable oxidation method is to treat compound of formula (XXVIII) with MnO₂ in a suitable solvent. Suitable solvents include dichloromethane, chloroform, dichloroethane and the like.

Several additional oxidation methods known to those skilled in the art are suitable for this oxidation.

A compound of formula (XXVIII) can be prepared from a compound of formula (XXVI).

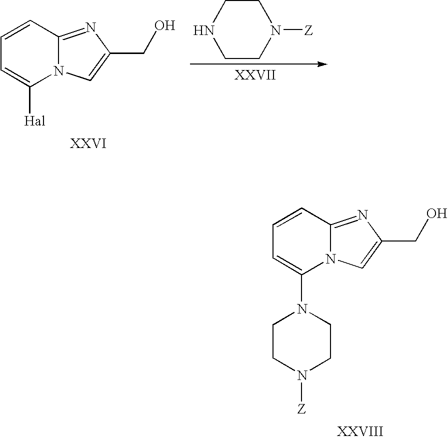

Treatment of compound of formula (XXVI) with piperazine of formula (XXVII) optionally in a suitable solvent optionally with heating or in a microwave can be used to give compound of formula (XXVIII). Alternatively the piperazine derivative (XXVII) can be treated with a strong base, such as n-BuLi or LDA, to form a salt. Treatment of a compound of formula (XXVI) with such a salt in a suitable solvent, such as tetrahydrofuran, can be used to form compound of formula (XXVIII). Compound of formula (XXVI) can be prepared as outlined in Scheme 10 by reduction of aldehyde of formula (XXV). Aldehydes of formula (XXV) can be prepared in a similar fashion as described in the literature (e.g. Tetrahedron 2002, 58, 489).

A compound of formula (I-F) where R^{a}OR⁵ is CH₂OH, n and m are 0, t is 1, and X—Yₚ is a piperazine suitably substituted with Z, where Z is C₁-C₆alkyl or C₃-C₈cycloalkyl and all other variables are as defined in connection with compound of formula (I-G) can be synthesized in a chiral fashion as outlined in Scheme 11.

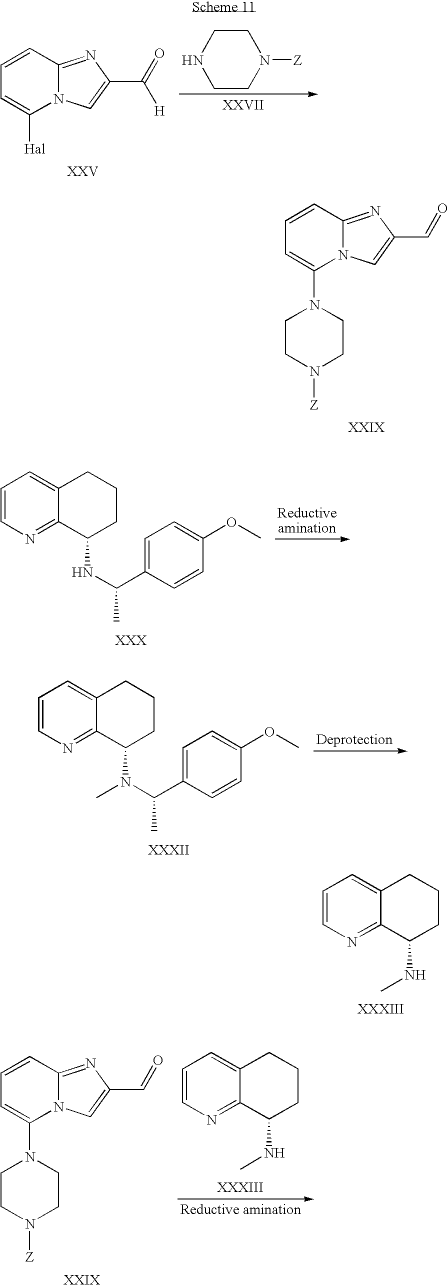

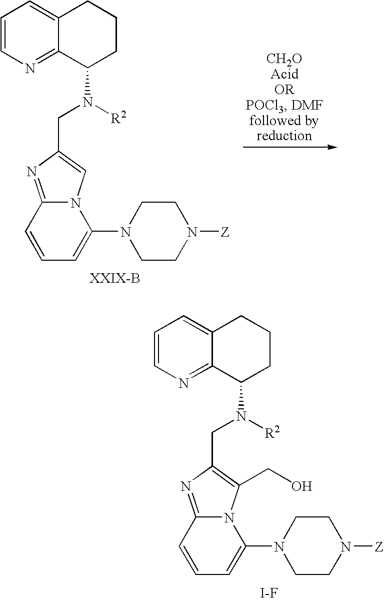

Compound of formula (I-F) can be prepared from compounds of formula (XXIX-B) by hydroxymethylation as described in connection with previous Schemes.

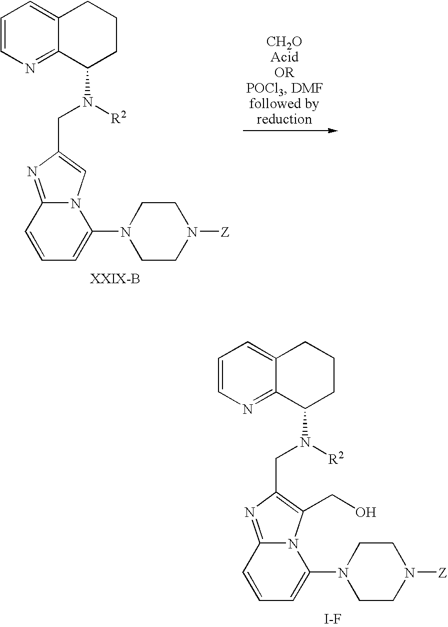

Compound of formula (XXIX-B) can be prepared from compounds of formula (XXIX) and (XXXIII) via reductive amination.

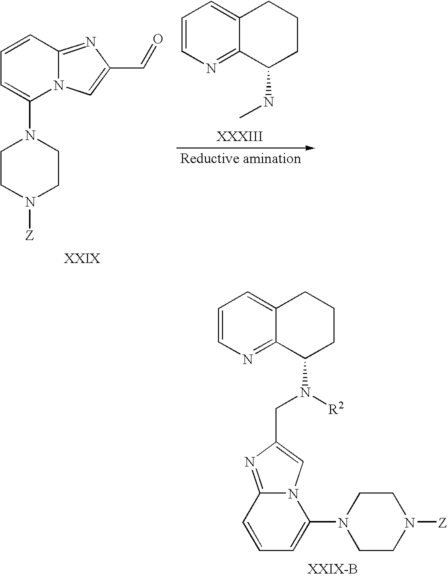

The reductive amination can be carried out in an inert solvent in the presence of a reducing agent. The reaction may be heated to 50-150° C. or performed at ambient temperature. Suitable solvents include dichloromethane, dichloroethane, tetrahydrofuran, acetonitrile, toluene, and the like. The reducing agent is typically sodium borohydride, sodium cyanoborohydride, sodium triacetoxyborohydride, and the like. Optionally the reaction can be run in presence of acid, such as acetic acid and the like. A compound of formula (XXXIII) can be prepared from a compound of formula (XXX) by reductive amination followed by deprotection using conditions similar to those described in connection with Scheme 10. Compounds of formula (XXIX) can be prepared in a similar fashion as described in connection with Scheme 10. As is evident to one skilled in the art the other enantiomer can be made in a similar fashion.

### EXAMPLES

### Example 1

### 5-Fluoroimidazo[1,2-a]pyridine-2-carbaldehyde (Intermediate)

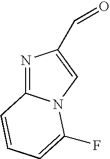

To a solution of 6-fluoro-2-pyridinamine (Tetrahedron, 2002, 58, 489, incorporated by reference with regard to such) (2.8 g, 25 mmol) in ethylene glycol dimethyl ether (28 mL) was added trichloroacetone (7.9 mL, 75 mmol). The mixture was stirred at room temperature for 15 hours and the resulting precipitate was collected by filtration and refluxed in ethyl alcohol (8 mL) for 4 hours. The reaction mixture was cooled to room temperature, concentrated, dissolved in dichloromethane and washed with saturated aqueous sodium bicarbonate. The organic layer was isolated, dried with magnesium sulfate, and concentrated. The resulting solid was refluxed in aqueous calcium carbonate for 2 hours, cooled to room temperature, and extracted with dichloromethane. The organic layer was dried with magnesium sulfate and concentrated to give 1.4 g (34% yield) 5-fluoroimidazo[1,2-a]pyridine-2-carbaldehyde as a tan solid. ¹H-NMR (CDCl₃): δ 10.16 (s, 1H), 8.22 (s, 1H), 7.54 (d, 1H), 7.34 (m, 1H), 6.59 (m, 1H); TLC (10% 2 M ammonia in methyl alcohol-ethyl acetate) R_{f}=0.60.

### Example 2

### 5-Bromoimidazo[1,2-a]pyridine-2-carbaldehyde (Intermediate)

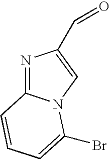

To a solution of 2-amino-6-bromopyridine (10 g, 58 mmol) in ethylene glycol dimethyl ether (66 mL) was added trichloroacetone (18 mL, 173 mmol). The mixture was stirred at 70° C. for 15 hours and the resulting precipitate was collected by filtration and refluxed in ethyl alcohol (50 mL) for 7 hours. The reaction mixture was cooled to room temperature, concentrated, dissolved in dichloromethane, and washed with saturated aqueous sodium bicarbonate. The organic layer was isolated, dried with magnesium sulfate, and concentrated. The resulting solid was refluxed in aqueous calcium carbonate for 1.5 hours, cooled to room temperature, and extracted with dichloromethane. The organic layer was dried with magnesium sulfate and concentrated to give 6.6 g (50% yield) 5-bromoimidazo[1,2-a]pyridine-2-carbaldehyde as an orange solid. ¹H-NMR (CDCl₃): δ 10.16 (s, 1H), 8.37 (s, 1H), 7.69 (d, 1H), 7.22 (m, 1H), 7.16 (m, 1H); TLC (10% ammonium hydroxide-acetonitrile) R_{f}=0.44.

### Example 3

### N-Methyl-5,6,7,8-tetrahydro-8-quinolinamine (Intermediate)

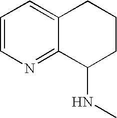

To a solution of 6,7-dihydro-8(5H)-quinolinone included in general processes above (1.5 g, 10 mmol) in dichloroethane (50 mL) was added methyl amine (2 M in tetrahydrofuran, 10 mL, 20 mmol), acetic acid (580 μL, 10 mmol), and sodium triacetoxyborohydride (4.3 g, 20 mmol). The mixture was stirred at room temperature for 15 hours and then filtered through a silica plug and rinsed with 10% ammonium hydroxide-acetonitrile. The solvent was removed and the residue purified by flash chromatography (0-10% ammonium hydroxide-acetonitrile) to give 1.4 g (85% yield) N-methyl-5,6,7,8-tetrahydro-8-quinolinamine as a yellow oil. ¹H-NMR (CDCl₃): δ 8.37 (d, 1H), 7.36 (d, 1H), 7.05 (t, 1H), 3.64 (t, 1H), 2.75 (m, 2H), 2.52 (s, 3H), 2.11 (m, 1H), 1.96 (m, 1H), 1.75 (m, 2H); MS m/z 163 (M+1).

### Example 4

### N-[(5-Fluoroimidazo[1,2-a]pyridin-2-yl)methyl]-N-methyl-5,6,7,8-tetrahydro-8-quinolinamine (Intermediate)

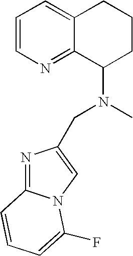

To a solution of N-methyl-5,6,7,8-tetrahydro-8-quinolinamine (340 mg, 2.1 mmol) and 5-fluoroimidazo[1,2-a]pyridine-2-carbaldehyde (344 mg, 2.1 mmol) in dichloroethane (10 mL) was added acetic acid (120 μL, 2.1 mmol) and sodium triacetoxyborohydride (1.3 g, 6.3 mmol). The mixture was stirred at room temperature for 2 hours and then filtered through a silica plug and rinsed with 10% ammonium hydroxide-acetonitrile. The solvent was removed and the residue purified by flash chromatography (0-10% ammonium hydroxide-acetonitrile) to give 0.6 g (93% yield) N-[(5-fluoroimidazo[1,2-a]pyridin-2-yl)methyl]-N-methyl-5,6,7,8-tetrahydro-8-quinolinamine as a tan solid. ¹H-NMR (CDCl₃): δ 8.53 (d, 1H), 7.80 (s, 1H), 7.36 (m, 2H), 7.13 (m, 1H), 7.06 (m, 1H), 6.40 (m, 1H), 4.10 (m, 1H), 3.94 (s, 2H), 2.75 (m, 2H), 2.43 (s, 3H), 2.03 (m, 3H), 1.70 (m, 1H); MS m/z 311 (M+1).

### Example 5

### N-[(5-Bromoimidazo[1,2-a]pyridin-2-yl)methyl]-N-methyl-5,6,7,8-tetrahydro-8-quinolinamine (Intermediate)

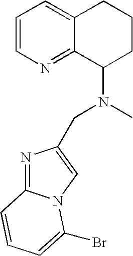

To a solution of N-methyl-5,6,7,8-tetrahydro-8-quinolinamine (500 mg, 3.1 mmol) and 5-bromoimidazo[1,2-a]pyridine-2-carbaldehyde (770 mg, 3.4 mmol) in dichloroethane (17 mL) was added acetic acid (180 μL, 3.1 mmol) and sodium triacetoxyborohydride (2.0 g, 9.3 mmol). The mixture was stirred at room temperature for 15 hours and then filtered through a silica plug and rinsed with 10% ammonium hydroxide-acetonitrile. The solvent was removed and the residue diluted with dichloromethane, washed with saturated aqueous sodium bicarbonate, and dried with magnesium sulfate to give 1.1 g (99% yield) of N-[(5-bromoimidazo[1,2-a]pyridin-2-yl)methyl]-N-methyl-5,6, 7,8-tetrahydro-8-quinolinamine as an orange oil. ¹H-NMR (CDCl₃): δ 8.50 (d, 1H), 7.92 (s, 1H), 7.49 (d, 1H), 7.32 (d, 1H), 7.03 (m, 2H), 6.96 (m, 1H), 4.09 (m, 1H), 3.94 (s, 2H), 2.72 (m, 2H), 2.40 (s, 3H), 2.12 (m, 1H), 1.99 (m, 2H), 1.68 (m, 1H); MS m/z 372 (M+1).

### Example 6

### N-Methyl-N-{[5-(4-methyl-1-piperazinyl)imidazo[1,2-a]pyridin-2-yl]methyl}-5,6, 7,8-tetrahydro-8-quinolinamine (Intermediate)

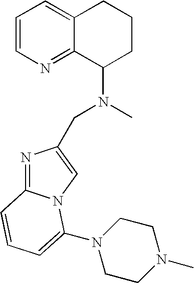

A solution of N-[(5-fluoroimidazo[1,2-a]pyridin-2-yl)methyl]-N-methyl-5,6,7,8-tetrahydro-8-quinolinamine (150 mg, 0.48 mmol) in neat 1-methylpiperazine (270 μL, 2.4 mmol) was subjected to microwave irradiation at 200° C. for 20 minutes. The reaction mixture was concentrated and purified by preparative chromatography (0-30% acetonitrile-water; 0.1% trifluoroacetic acid) and then diluted with ethyl acetate, washed with saturated aqueous sodium bicarbonate, and dried with magnesium sulfate to give 125 mg (67% yield) of a yellow oil. ¹H-NMR (CDCl₃): δ 8.52 (d, 1H), 7.70 (s, 1H), 7.34 (m, 1H), 7.28 (m, 1H), 7.10 (m, 1H), 7.04 (m, 1H), 6.23 (dd, 1H), 4.13 (m, 1H), 3.96 (m, 2H), 3.13 (s, 4H), 2.82 (m, 2H), 2.65 (s, 4H), 2.40 (s, 6H), 2.01 (m, 3H), 1.70 (m, 1H); MS m/z 391 (M+1).

Alternatively:

N-Methyl-N-{[5-(4-methyl-1-piperazinyl)imidazo[1,2-a]pyridin-2-yl]methyl}-5,6,7,8-tetrahydro-8-quinolinamine can also be prepared by reductive amination. A solution of N-methyl-5,6,7,8-tetrahydro-8-quinolinamine (44 mg, 0.27 mmol) and 5-(4-methyl-1-piperazinyl)imidazo[1,2-a]pyridine-2-carbaldehyde (69 mg, 0.30 mmol) in dichloroethane (1.4 mL) was treated with glacial acetic acid (15 μL, 0.27 mmol) and sodium triacetoxyborohydride (172 mg, 0.81 mmol). The mixture was stirred at room temperature for 15 hours and then filtered through a silica plug and rinsed with 10% 2M ammonia in methanol-ethyl acetate. The reaction mixture was concentrated and purified by preparative chromatography (0-70% acetonitrile-water; 0.1% trifluoroacetic acid) and then diluted with ethyl acetate, washed with saturated aqueous sodium bicarbonate, and dried with magnesium sulfate to give 9 mg (9% yield) of a yellow oil.

This racemic compound can also be separated by SFC to give the R and S isomers. Racemic N-methyl-N-{[5-(4-methyl-1-piperazinyl)imidazo[1,2-a]pyridin-2-yl]methyl}-5,6,7,8-tetrahydro-8-quinolinamine was separated into R and S isomers on a Berger analytical SFC with an HP1100 diode array detector. The sample was monitored at 230 nm under the following conditions: 15% co-solvent (50/50 MeOH/CHCl₃ with 0.5% diisopropylethylamine v/v) in CO₂ with a total flow rate of 2 mL/minute at 1500 psi, 27° C. on a Diacel AD-H column (Chiral Technologies), 4.6×250 mm, 5 um.

### Example 7

### N-{[5-(4-Ethyl-1-piperazinyl)imidazo[1,2-a]pyridin-2-yl]methyl}-N-methyl-5,6,7,8-tetrahydro-8-quinolinamine

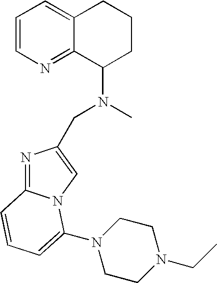

N-{[5-(4-Ethyl-1-piperazinyl)imidazo[1,2-a]pyridin-2-yl]methyl}-N-methyl-5,6,7,8-tetrahydro-8-quinolinamine was prepared from N-[(5-fluoroimidazo[1,2-a]pyridin-2-yl)methyl]-N-methyl-5,6,7,8-tetrahydro-8-quinolinamine and N-ethylpiperazine in a similar manner as described in Example 6 to give a yellow oil (24% yield). ¹H-NMR (CDCl₃): δ 8.49 (d, 1H), 7.66 (s, 1H), 7.32 (m, 1H), 7.27 (m, 1H), 7.08 (m, 1H), 7.03 (m, 1H), 6.21 (d, 1H), 4.11 (m, 1H), 3.94 (s, 2H), 3.12 (s, 4H), 2.79 (m, 2H), 2.67 (s, 4H), 2.51 (q, 2H), 2.33 (s, 3H), 2.01 (m, 3H), 1.66 (m, 1H), 1.12 (t, 3H); MS m/z 405 (M+1).

### Example 8

### N-Methyl-N-({5-[4-(1-methylethyl)-1-piperazinyl]imidazo[1,2-a]pyridin-2-yl}methyl)-5,6,7,8-tetrahydro-8-quinolinamine

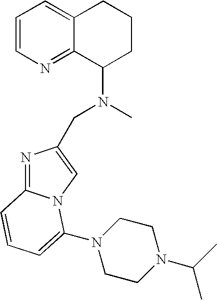

N-Methyl-N-({5-[4-(1-methylethyl)-1-piperazinyl]imidazo[1,2-a]pyridin-2-yl}methyl)-5,6, 7,8-tetrahydro-8-quinolinamine was prepared from N-[(5-fluoroimidazo[1,2-a]pyridin-2-yl)methyl]-N-methyl-5,6,7,8-tetrahydro-8-quinolinamine and isopropyl piperazine in a similar manner as described in Example 6 to give a yellow solid (12% yield). ¹H-NMR (CDCl₃): δ 8.53 (d, 1H), 7.74 (s, 1H), 7.35 (m, 1H), 7.28 (m, 1H), 7.11 (m, 1H), 7.05 (m, 1H), 6.23 (dd, 1H), 4.16 (m, 1H), 4.00 (m, 2H), 3.14 (s, 4H), 2.78 (m, 7H), 2.40 (s, 3H), 2.01 (m, 3H), 1.67 (m, 1H), 1.12 (d, 6H); MS m/z 419 (M+1).

### Example 9

### 1,1-Dimethylethyl 4-(2-{[methyl(5,6,7,8-tetrahydro-8-quinolinyl)amino]methyl}imidazo[1,2-a]pyridin-5-yl)-1-piperazinecarboxylate

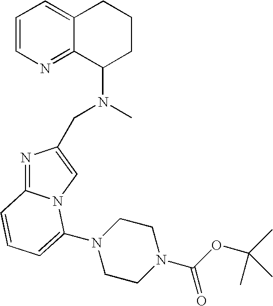

1,1-Dimethylethyl 4-(2-{[methyl(5,6,7,8-tetrahydro-8-quinolinyl)amino]methyl}imidazo[1,2-a]pyridin-5-yl)-1-piperazinecarboxylate was prepared from N-[(5-fluoroimidazo[1,2-a]pyridin-2-yl)methyl]-N-methyl-5,6,7,8-tetrahydro-8-quinolinamine and N-tert-butoxycarbonyl piperazine in a similar manner as described in Example 6 to give a tan solid (21% yield). ¹H-NMR (CDCl₃): δ 8.51 (d, 1H), 7.75 (s, 1H), 7.35 (m, 1H), 7.30 (m, 1H), 7.11 (m, 1H), 7.06 (m, 1H), 6.22 (dd, 1H), 4.17 (m, 1H), 4.02 (m, 2H), 3.66 (s, 4H), 3.04 (s, 4H), 2.81 (m, 2H), 2.39 (s, 3H), 2.03 (m, 3H), 1.69 (m, 1H), 1.49 (s, 9H); MS m/z 477 (M+1).

### Example 10

### N-Methyl-N-{[5-(1-piperazinyl)imidazo[1,2-a]pyridin-2-yl]methyl}-5,6,7,8-tetrahydro-8-quinolinamine

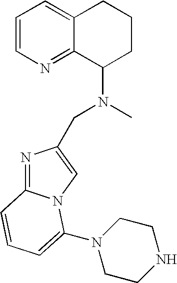

To a solution of 1,1-dimethylethyl 4-(2-{[methyl(5,6,7,8-tetrahydro-8-quinolinyl)amino]methyl}imidazo[1,2-a]pyridin-5-yl)-1-piperazinecarboxylate (20 mg, 0.04 mmol) in dichloromethane (300 μL) was added trifluoroacetic acid (300 μL). The mixture was stirred at room temperature for 2 hours, concentrated, diluted in 3:1 dichloromethane:isopropyl alcohol, washed with saturated aqueous sodium bicarbonate, and dried with magnesium sulfate to give 16 mg (100% yield) of a tan solid. ¹H-NMR (CDCl₃): δ 8.50 (d, 1H), 7.74 (s, 1H), 7.35 (m, 1H), 7.28 (d, 1H), 7.12 (m, 1H), 7.05 (m, 1H), 6.25 (d, 1H), 4.22 (m, 1H), 4.08 (m, 2H), 3.15 (m, 8H), 2.75 (m, 2H), 2.40 (s, 3H), 2.04 (m, 3H), 1.68 (m, 1H); MS m/z 377 (M+1).

### Example 11

### N,N,N′-Trimethyl-N′-(2-{[methyl(5,6,7,8-tetrahydro-8-quinolinyl)amino]methyl}imidazo[1,2-a]pyridin-5-yl)-1,2-ethanediamine

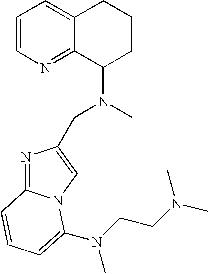

N,N,N′-Trimethyl-N′-(2-{[methyl(5,6,7,8-tetrahydro-8-quinolinyl)amino]methyl}imidazo[1,2-a]pyridin-5-yl)-1,2-ethanediamine was prepared from N-[(5-fluoroimidazo[1,2-a]pyridin-2-yl)methyl]-N-methyl-5,6,7,8-tetrahydro-8-quinolinamine and N,N,N′-trimethylethylenediamine in a similar manner as described in Example 6 to give a yellow oil (32% yield). ¹H-NMR (CDCl₃): δ 8.48 (d, 1H), 7.72 (s, 1H), 7.33 (m, 1H), 7.24 (m, 1H), 7.08 (m, 1H), 7.02 (m, 1H), 6.24 (dd, 1H), 4.09 (m, 1H), 3.94 (s, 2H), 3.16 (t, 2H), 2.83 (s, 3H), 2.68 (m, 2H), 2.52 (t, 2H), 2.32 (s, 3H), 2.08 (m, 3H), 1.67 (m, 1H); MS m/z 393 (M+1).

### Example 12

### N-{[5-(3,5-Dimethyl-1-piperazinyl)imidazo[1,2-a]pyridin-2-yl]methyl}-N-methyl-5,6, 7,8-tetrahydro-8-quinolinamine

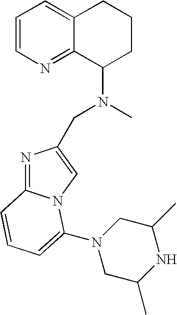

N-{[5-(3,5-Dimethyl-1-piperazinyl)imidazo[1,2-a]pyridin-2-yl]methyl}-N-methyl-5,6,7,8-tetrahydro-8-quinolinamine was prepared from N-[(5-fluoroimidazo[1,2-a]pyridin-2-yl)methyl]-N-methyl-5,6,7,8-tetrahydro-8-quinolinamine and 2,6-dimethylpiperazine in a similar manner as described in Example 6 to give a yellow oil (64% yield). ¹H-NMR (CDCl₃): δ 8.49 (d, 1H), 7.68 (s, 1H), 7.31 (m, 1H), 7.25 (m, 1H), 7.08 (m, 1H), 7.02 (m, 1H), 6.20 (dd, 1H), 4.12 (m, 1H), 3.96 (s, 2H), 3.28 (m, 2H), 3.18 (m, 2H), 2.73 (m, 2H), 2.34 (s, 3H), 2.27 (m, 2H), 2.04 (m, 3H), 1.65 (m, 1H), 1.11 (d, 3H), 1.01 (d, 3H); MS m/z 405 (M+1).

### Example 13

### N-Methyl-N-{[5-(3,4,5-trimethyl-1-piperazinyl)imidazo[1,2-a]pyridin-2-yl]methyl}-5,6,7,8-tetrahydro-8-quinolinamine

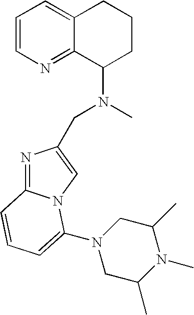

To a solution of N-{[5-(3,5-Dimethyl-1-piperazinyl)imidazo[1,2-a]pyridin-2-yl]methyl}-N-methyl-5,6,7,8-tetrahydro-8-quinolinamine (40 mg, 0.10 mmol) in THF (1 mL) at 0° C. was added sodium hydride (60% in oil, 10 mg, 0.15 mmol). The reaction was stirred for 10 minutes, treated with methyl iodide (5 μL, 0.50 mmol), and stirred at room temperature overnight. The reaction was quenched with saturated aqueous sodium bicarbonate, extracted into 3:1 dichloromethane:isopropyl alcohol, dried with magnesium sulfate, filtered, and concentrated. The residue was purified by preparative chromatography (0-40% acetonitrile-water; 0.1% trifluoroacetic acid) and then diluted with 3:1 dichloromethane:isopropyl alcohol, washed with saturated aqueous sodium bicarbonate, and dried with magnesium sulfate to give 12 mg (29% yield) of a yellow oil. ¹H-NMR (CDCl₃): δ 8.52 (d, 1H), 7.71 (s, 1H), 7.34 (m, 1H), 7.27 (m, 1H), 7.10 (m, 1H), 7.05 (m, 1H), 6.21 (dd, 1H), 4.15 (m, 1H), 3.99 (s, 2H), 3.28 (m, 2H), 2.62 (m, 6H), 2.38 (s, 3H), 2.36 (s, 3H), 2.07 (m, 3H), 1.66 (m, 1H), 1.15 (d, 3H), 1.06 (d, 3H); MS m/z 419 (M+1).

### Example 14

### N-(1-Methylethyl)-N-{[5-(4-methyl-1-piperazinyl)imidazo[1,2-a]pyridin-2-yl]methyl}-5,6,7,8-tetrahydro-8-quinolinamine

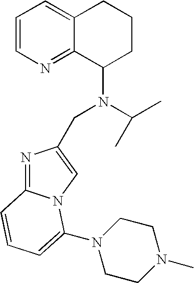

N-(1-Methylethyl)-N-{[5-(4-methyl-1-piperazinyl)imidazo[1,2-a]pyridin-2-yl]methyl}-5,6,7,8-tetrahydro-8-quinolinamine was prepared from N-[(5-fluoroimidazo[1,2-a]pyridin-2-yl)methyl]-N-(1-methylethyl)-5,6,7,8-tetrahydro-8-quinolinamine and 1-methylpiperazine in a similar manner as described in Example 6 to give a yellow oil (39% yield). ¹H-NMR (CDCl₃): δ 8.44 (d, 1H), 7.64 (s, 1H), 7.22 (m, 1H), 7.19 (m, 1H), 7.05 (m, 1H), 6.95 (m, 1H), 6.18 (dd, 1H), 4.21 (m, 1H), 3.93 (m, 2H), 3.16 (m, 1H), 3.10 (s, 4H), 2.75 (m, 2H), 2.67 (s, 4H), 2.41 (s, 3H), 1.98 (m, 3H), 1.62 (m, 1H), 1.12 (dd, 6H); MS m/z 419 (M+1).

### Example 15

### N-(1-Methylethyl)-N-({5-[4-(1-methylethyl)-1-piperazinyl]imidazo[1,2-a]pyridin-2-yl}methyl)-5,6,7,8-tetrahydro-8-quinolinamine

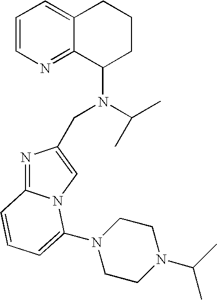

N-(1-Methylethyl)-N-({5-[4-(1-methylethyl)-1-piperazinyl]imidazo[1,2-a]pyridin-2-yl}methyl)-5,6,7,8-tetrahydro-8-quinolinamine was prepared from N-[(5-fluoroimidazo[1,2-a]pyridin-2-yl)methyl]-N-(1-methylethyl)-5,6,7,8-tetrahydro-8-quinolinamine and isopropyl piperazine in a similar manner as described in Example 6 to give a yellow oil (43% yield). ¹H-NMR (CDCl₃): δ 8.44 (d, 1H), 7.65 (s, 1H), 7.22 (m, 1H), 7.19 (m, 1H), 7.05 (m, 1H), 6.96 (m, 1H), 6.17 (dd, 1H), 4.22 (m, 1H), 3.94 (m, 2H), 3.16 (m, 1H), 3.10 (s, 4H), 2.77 (m, 5H), 2.62 (m, 2H), 2.01 (m, 3H), 1.63 (m, 1H), 1.12 (m, 12H); MS m/z 447 (M+1).

### Example 16

### N-{[5-(4-Methyl-1-piperazinyl)imidazo[1,2-a]pyridin-2-yl]methyl}-N-(2, 2,2-trifluoroethyl)-5,6,7,8-tetrahydro-8-quinolinamine

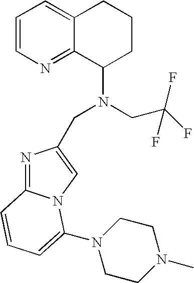

N-{[5-(4-Methyl-1-piperazinyl)imidazo[1,2-a]pyridin-2-yl]methyl}-N-(2,2,2-trifluoroethyl)-5,6,7,8-tetrahydro-8-quinolinamine was prepared from N-[(5-fluoroimidazo[1,2-a]pyridin-2-yl)methyl]-N-(2,2,2-trifluoroethyl)-5,6,7,8-tetrahydro-8-quinolinamine and 1-methylpiperazine in a similar manner as described in Example 6 to give a yellow oil (48% yield). ¹H-NMR (CDCl₃): δ 8.41 (d, 1H), 7.61 (s, 1H), 7.32 (m, 1H), 7.23 (m, 1H), 7.10 (m, 1H), 7.04 (m, 1H), 6.23 (dd, 1H), 4.27 (m, 1H), 4.21 (m, 1H), 3.99 (m, 2H), 3.94 (m, 1H), 3.31 (m, 1H), 3.15 (m, 4H), 2.70 (m, 5H), 2.41 (s, 3H), 2.20 (m, 1H), 1.94 (m, 1H), 1.78 (m, 2H); MS m/z 459 (M+1).

### Example 17

### N-({5-[4-(1-Methylethy)-1-piperazinyl]imidazo[1,2-a]pyridin-2-yl}methyl)-N-(2, 2,2-trifluoroethyl)-5,6,7,8-tetrahydro-8-quinolinamine

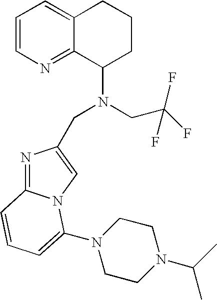

N-({5-[4-(1-Methylethyl)-1-piperazinyl]imidazo[1,2-a]pyridin-2-yl}methyl)-N-(2,2,2-trifluoroethyl)-5,6,7,8-tetrahydro-8-quinolinamine was prepared from N-[(5-fluoroimidazo[1,2-a]pyridin-2-yl)methyl]-N-(2,2,2-trifluoroethyl)-5,6,7,8-tetrahydro-8-quinolinamine and isopropyl piperazine in a similar manner as described in Example 6 to give a yellow oil (43% yield). ¹H-NMR (CDCl₃): δ 8.41 (d, 1H), 7.61 (s, 1H), 7.32 (m, 1H), 7.22 (m, 1H), 7.10 (m, 1H), 7.04 (m, 1H), 6.22 (dd, 1H), 4.26 (m, 1H), 4.22 (m, 1H), 3.96 (m, 2H), 3.31 (m, 1H), 3.14 (m, 4H), 2.78 (m, 7H), 2.20 (m, 1H), 1.95 (m, 1H), 1.78 (m, 2H), 1.13 (d, 6H); MS m/z 487 (M+1).

### Example 18

### N-[(5-{4-[(Dimethylamino)methyl]phenyl}imidazo[1,2-a]pyridin-2-yl)methyl]-N-methyl-5,6,7,8-tetrahydro-8-quinolinamine

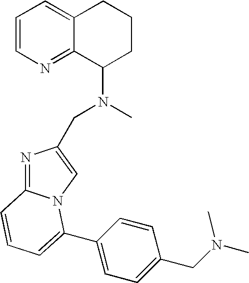

To a suspension of N-[(5-bromoimidazo[1,2-a]pyridin-2-yl)methyl]-N-methyl-5,6,7,8-tetrahydro-8-quinolinamine (75 mg, 0.20 mmol), potassium carbonate (140 mg, 1.02 mmol), and 4-(N,N-dimethylaminomethyl)phenyl boronic acid pinacol ester (120 mg, 0.46 mmol) in ethylene glycol dimethyl ether (2.9 mL) was added tetrakis(triphenylphosphine) palladium(0) (86 mg, 0.074 mmol) and water (0.11 mL). The reaction was heated at 80° C. for 15 hours, diluted with water, extracted into ethyl acetate, concentrated, and purified by preparative chromatography (0-50% acetonitrile-water; 0.1% trifluoroacetic acid). The purified product was then diluted with ethyl acetate, washed with saturated aqueous sodium bicarbonate, and dried with magnesium sulfate to give 30 mg (35% yield) of a yellow oil. ¹H-NMR (CDCl₃): δ 8.40 (d, 1H), 7.79 (s, 1H), 7.57 (m, 1H), 7.55 (m, 1H), 7.50 (m, 1H), 7.45 (m, 1H), 7.43 (m, 1H), 7.31 (m, 1H), 7.17 (m, 1H), 7.00 (m 1H), 6.67 (dd, 1H), 4.07 (m, 1H), 3.90 (s, 2H), 3.50 (s, 2H), 2.72 (m, 2H), 2.31 (s, 3H), 2.29 (s, 6H), 2.10 (m, 1H), 1.99 (m, 2H), 1.64 (m, 1H); MS m/z 426 (M+1).

### Example 19

### N-Methyl-N-{[5-(4-pyridinyl)imidazo[1,2-a]pyridin-2-yl]methyl}-5,6,7,8-tetrahydro-8-quinolinamine

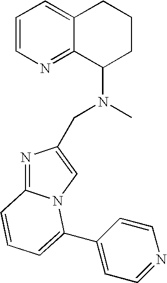

N-Methyl-N-{[5-(4-pyridinyl)imidazo[1,2-a]pyridin-2-yl]methyl}-5,6,7,8-tetrahydro-8-quinolinamine was prepared from N-[(5-bromoimidazo[1,2-a]pyridin-2-yl)methyl]-N-methyl-5,6, 7,8-tetrahydro-8-quinolinamine and pyridine-4-boronic acid in a similar manner as described above to give a yellow oil (8% yield). ¹H-NMR (CDCl₃): δ 8.80 (m, 2H), 8.44 (m, 1H), 7.96 (s, 1H), 7.59 (m, 3H), 7.35 (d, 1H), 7.22 (m, 1H), 7.05 (m, 1H), 6.77 (m, 1H), 4.11 (m, 1H), 3.96 (s, 2H), 2.75 (m, 2H), 2.39 (s, 3H), 2.15 (m, 1H), 1.99 (m, 2H), 1.68 (m, 1H); MS m/z 370 (M+1).

### Example 20

### (5-(4-Methyl-1-piperazinyl)-2-{[methyl(5,6,7,8-tetrahydro-8-quinolinyl)amino]methyl}imidazo[1,2-a]pyridin-3-yl)methanol

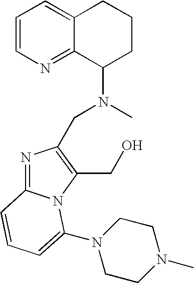

(5-(4-Methyl-1-piperazinyl)-2-{[methyl(5,6,7,8-tetrahydro-8-quinolinyl)amino]methyl}imidazo[1,2-a]pyridin-3-yl)methanol was prepared from N-methyl-N-{[5-(4-methyl-1-piperazinyl)imidazo[1,2-a]pyridin-2-yl]methyl}-5,6,7,8-tetrahydro-8-quinolinamine via hydroxymethylation in a similar manner as shown herein to give a white solid (30% yield). ¹H NMR (400 MHz, CDCl₃) δ 8.42 (m, 1H), 7.33-7.29 (m, 2H), 7.07-6.99 (m, 2H), 6.39 (d, J=7.1 Hz, 1H), 5.28 (s, 2H), 4.08-3.95 (m, 3H), 3.51 (d, J=10.1 Hz, 1H), 3.37 (d, J=10.6 Hz, 1H), 2.89 (m, 4H), 2.76 (m, 1H), 2.66 (m, 1H), 2.55-2.47 (m, 2H), 2.39 (s, 3H), 2.21 (m, 1H), 2.13 (s, 3H), 2.02-1.88 (m, 2H), 1.67 (m, 1H); MS m/z 421 (M+1).

### Example 21

### (8S)—N-[(1S)-1-[4-(Methyloxy)phenyl]ethyl]-N-{[5-(4-methyl-1-piperazinyl)imidazo[1,2-a]pyridin-2-yl]-methyl}-5,6,7,8-tetrahydro-8-quinolinamine (Intermediate)

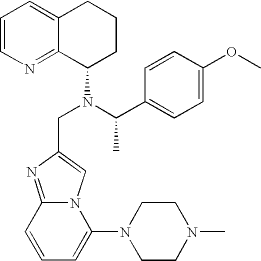

### A) 6-Fluoro-2-Pyridinamine

A solution of 2,6-difluoropyridine (50 g, 434 mmol) in ammonium hydroxide (200 mL, 28.0-30.0%) was heated at 105° C. in a steel bomb for 15 hours. The reaction was cooled in an ice bath and the precipitate filtered, rinsed with cold water, and dried to yield 6-fluoro-2-pyridinamine (45.8 g, 94% yield) as a white solid. ¹H-NMR (CDCl₃): δ 7.53 (m, 1H), 6.36 (dd, 1H), 6.26 (dd, 1H), 4.56 (s, 2H).

### B) 2-(Dichloromethyl)-5-fluoroimidazo[1,2-a]pyridine

A solution of 6-fluoro-2-pyridinamine (67 g, 0.60 mol) in ethylene glycol dimethyl ether (570 mL) was treated with 1,1,3-trichloroacetone (190 mL, 1.80 mol) and heated at 85° C. for 15 hours. The reaction was cooled in an ice bath and the precipitate filtered, rinsed with hexanes, and dried to yield 2-(dichloromethyl)-5-fluoroimidazo[1,2-a]pyridine (85 g, 65% yield) as an olive green solid. ¹H-NMR (CDCl₃): δ 8.18 (s, 1H), 7.60 (s, 1H), 7.54-7.46 (m, 2H), 6.93 (m, 1H).

### C) 5-Fluoroimidazo[1,2-a]pyridine-2-carbaldehyde

A solution of 2-(dichloromethyl)-5-fluoroimidazo[1,2-a]pyridine (103 g, 470 mmol) in ethanol (300 mL) and water (600 mL) was treated with sodium acetate (96 g, 1.17 mol) and heated at 60° C. for 2 hours. The reaction was cooled, filtered though celite, and concentrated in vacuo to remove the ethanol. The aqueous was extracted twice with chloroform and the organics were combined, washed with water and brine, dried over sodium sulfate, and concentrated. The residue was filtered through a pad of silica, rinsed with dichloromethane and ethyl acetate, concentrated, triturated with hexanes, filtered, and dried to yield 5-fluoroimidazo[1,2-a]pyridine-2-carbaldehyde (40 g, 52% yield) as a tan solid. ¹H-NMR (CDCl₃): δ 10.17 (s, 1H), 8.22 (s, 1H), 7.57 (d, 1H), 7.38-7.32 (m, 1H), 6.60 (m, 1H); TLC (10% 2 M ammonia in methy alcohol-ethyl acetate) R_{f}=0.60.

### D) (5-Fluoroimidazo[1,2-a]pyridin-2-yl)methanol

A solution of 5-fluoroimidazo[1,2-a]pyridine-2-carbaldehyde (80 g, 490 mmol) in methanol (1 L) at 0° C. was treated with sodium borohydride (24 g, 640 mmol) in portions. The reaction was slowly brought to room temperature, stirred for 2 hours, quenched with water, concentrated, dissolved in 3:1 dichloromethane to isopropyl alcohol, and washed with saturated aqueous sodium bicarbonate. The organic layer was separated and the aqueous extracted four times with 3:1 dichloromethane to isopropyl alcohol. The organic layers were combined, dried over sodium sulfate, concentrated, triturated with hexanes, and filtered to yield (5-fluoroimidazo[1,2-a]pyridin-2-yl)methanol (76 g, 93% yield) as a brown solid. ¹H-NMR (CDCl₃): δ 7.59 (s, 1H), 7.38 (d, 1H), 7.21-7.15 (m, 1H), 6.43 (m, 1H), 4.85 (s, 2H), 4.45 (s, 1H).

### E) [5-(4-Methyl-1-piperazinyl)imidazo[1,2-a]pyridin-2-yl]methanol

A solution of (5-fluoroimidazo[1,2-a]pyridin-2-yl)methanol (76 g, 460 mmol) in 1-methyl piperazine (150 mL) was heated at 70° C. for 15 hours. The reaction mixture was cooled, poured into 1.3 L brine, and extracted into 3:1 chloroform to isopropyl alcohol. The combined extracts were dried over sodium sulfate, concentrated in vacuo, azeotroped with hexanes, and triturated with diethyl ether to yield [5-(4-methyl-1-piperazinyl)imidazo[1,2-a]pyridin-2-yl]methanol (101 g, 90% yield) as a tan solid. ¹H-NMR (CDCl₃): δ 7.51 (s, 1H), 7.33 (d, 1H), 7.21-7.17 (m, 1H), 6.31 (m, 1H), 4.87 (s, 2H), 3.17 (s, 4H), 2.68 (s, 4H), 2.42 (s, 3H).

### F) 5-(4-Methyl-1-piperazinyl)imidazo[1,2-a]pyridine-2-carbaldehyde

A solution of [5-(4-methyl-1-piperazinyl)imidazo[1,2-a]pyridin-2-yl]methanol (101 g, 410 mmol) in chloroform (1650 mL) was treated with manganese dioxide (360 g, 4100 mmol) and stirred at room temperature for 72 hours. The reaction mixture was filtered through celite, rinsed with chloroform, and concentrated to yield 5-(4-methyl-1-piperazinyl)imidazo[1,2-a]pyridine-2-carbaldehyde (82 g, 82% yield) as gold solid. ¹H-NMR (CDCl₃): δ 10.17 (s, 1H), 8.15 (s, 1H), 7.44 (d, 1H), 7.31-7.27 (m, 1H), 6.40 (m, 1H), 3.16 (s, 4H), 2.68 (s, 4H), 2.42 (s, 3H).

### G) (8S)—N-{(1S)-1-[4-(Methyloxy)phenyl]ethyl}-5,6,7,8-tetrahydro-8-quinolinamine

A solution of (S)-(−)-1-(4-methoxyphenyl)ethylamine (25 g, 166 mmol) and 6,7-dihydro-8(5H)-quinolinone (24 g, 166 mmol) in dichloromethane was treated with glacial acetic acid (14 mL, 249 mmol) and sodium triacetoxyborohydride (53 g, 249 mmol). The reaction mixture was stirred at room temperature for 15 hours and treated with sodium carbonate (106 g, 996 mmol) and stirred for 30 minutes. The mixture was diluted with dichloromethane, the organic layer separated, and the aqueous extracted with more dichloromethane. The organic layers were combined, dried over magnesium sulfate, concentrated, and purified by column chromatography (0-3% 2 M ammonia in methanol/dichloromethane) to give a yellow oil which was crystallized from hexanes to yield (8S)—N-{(1S)-1-[4-(methyloxy)phenyl]ethyl}-5,6,7,8-tetrahydro-8-quinolinamine (33 g, 70% yield) as clear crystals. ¹H-NMR (CDCl₃): δ 8.40 (m, 1H), 7.33 (m, 3H), 7.04 (m, 1H), 6.84 (d, 2H), 4.02 (m, 1H), 3.83-3.78 (m, 4H), 2.73-2.62 (m, 2H), 1.82 (m, 1H), 1.72 (m, 1H), 1.57 (m, 2H), 1.43 (d, 3H).

### H) (8S)—N-[(1S)-1-[4-(Methyloxy)phenyl]ethyl]-N-{[5-(4-methyl-1-piperazinyl)imidazo[1,2-a]pyridin-2-yl]methyl}-5,6,7,8-tetrahydro-8-quinolinamine

A solution of 5-(4-methyl-1-piperazinyl)imidazo[1,2-a]pyridine-2-carbaldehyde (2.83 g, 11.6 mmol) and (8S)—N-{(1S)-1-[4-(methyloxy)phenyl]ethyl}-5,6,7,8-tetrahydro-8-quinolinamine (3.27 g, 11.6 mmol) in dichloroethane (40 mL) was treated with glacial acetic acid (1.0 mL, 17.4 mmol) and sodium triacetoxyborohydride (3.68 g, 17.4 mmol, added in portions) and stirred at room temperature for 15 hours. The reaction mixture was diluted with dichloromethane, washed with saturated aqueous sodium bicarbonate, separated, and extracted with additional dichloromethane. The organic layers were combined, washed with brine, dried over sodium sulfate, concentrated, and purified by flash chromatography (0-4% ammonium hydroxide in acetonitrile). The residue was dissolved in dichloromethane and stirred with 2 M ammonia in methanol to yield (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine (5.13 g, 87% yield) as pale yellow foam.

¹H NMR (400 MHz, CDCl₃) δ 8.48 (d, J=4.6 Hz, 1H), 7.78 (s, 1H), 7.60-7.58 (m, 2H), 7.24-7.18 (m, 2H), 7.09-7.05 (m, 1H), 6.97 (dd, J=7.6, 4.7 Hz, 1H), 6.84-6.82 (m, 2H), 6.21 (d, J=7.2 Hz, 1H), 4.82 (m, 1H), 4.07 (m, 1H), 3.91 (dd, J=56.9, 17.1 Hz, 2H), 3.77 (s, 3H), 3.19-3.13 (m, 4H), 2.74 (s, 4H), 2.67-2.53 (m, 2H), 2.47 (s, 3H), 2.06 (m, 1H), 1.85 (m, 2H), 1.53 (m, 1H), 1.34 (d, J=6.4 Hz, 3H); MS m/z 511 (M+1).

### Example 22

### [2-({{(1S)-1-[4-(Methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol

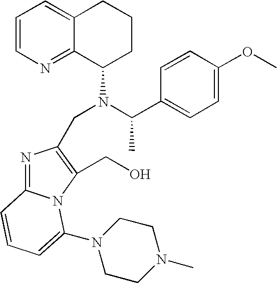

[2-({{(1S)-1-[4-(Methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol was prepared from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine via hydroxymethylation in a similar manner as shown herein to give an off-white solid (80% yield). ¹H NMR (400 MHz, CDCl₃) δ 8.42 (d, J=4.2 Hz, 1H), 7.43 (d, J=8.6 Hz, 2H), 7.28-7.21 (m, 2H), 7.05-7.01 (m, 1H), 6.95 (dd, J=7.6, 4.8 Hz, 1H), 6.82-6.80 (m, 2H), 6.38 (d, J=7.1 Hz, 1H), 5.19 (d, J=12.9 Hz, 1H), 4.61 (d, J=12.9 Hz, 1H), 4.09-4.06 (m, 2H), 4.03-3.93 (m, 2H), 3.77 (s, 3H), 3.63 (m, 2H), 3.08 (m, 1H), 2.92-2.80 (m, 4H), 2.71 (m, 1H), 2.59-2.52 (m, 2H), 2.47-2.44 (m, 2H), 2.40 (s, 3H), 2.32 (m, 1H), 2.16 (m, 1H), 1.96 (m, 1H), 1.55 (d, J=7.1 Hz, 3H); MS m/z 541 (M+1).

### Example 23

### (8S)—N-{[5-(4-methyl-1-piperazinyl)imidazo[1,2-a]pyridin-2-yl]methyl}-5,6,7,8-tetrahydro-8-quinolinamine (Intermediate)

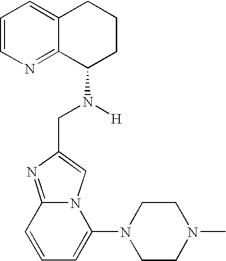

A solution of (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine (569 mg, 1.11 mmol) in dichloromethane (11.1 mL) was treated with trifluoroacetic acid (1.11 mL) and stirred at room temperature for 4 hours. The reaction was concentrated, diluted with dichloromethane, and washed with saturated aqueous sodium bicarbonate. The organic layer was separated and the aqueous extracted with dichloromethane. The organic layers were combined, dried over magnesium sulfate, filtered, and concentrated to yield (8S)—N-{[5-(4-methyl-1-piperazinyl)imidazo[1,2-a]pyridin-2-yl]methyl}-5,6,7,8-tetrahydro-8-quinolinamine as a yellow residue. ¹H-NMR (CDCl₃): δ 8.41 (d, 1H), 7.65 (s, 1H), 7.39 (d, 1H), 7.31 (m, 1H), 7.16 (m, 1H), 7.09 (m, 1H), 6.27 (dd, 1H), 4.31-4.17 (m, 2H), 4.05 (m, 1H), 3.15 (m, 4H), 2.88-2.78 (m, 2H), 2.67 (m, 4H), 2.41 (s, 3H), 2.29 (m, 1H), 2.08 (m, 1H), 1.96 (m, 1H), 1.77 (m, 1H).

### Example 24

### (8S)—N-Methyl-N-{[5-(4-methyl-1-piperazinyl)imidazo[1,2-a]pyridin-2-yl]methyl}-5,6,7,8-tetrahydro-8-quinolinamine (Intermediate)

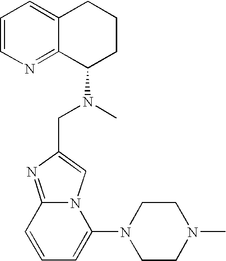

(8S)—N-{[5-(4-methyl-1-piperazinyl)imidazo[1,2-a]pyridin-2-yl]methyl}-5,6,7,8-tetrahydro-8-quinolinamine was dissolved in dichloroethane (10 mL) and treated with formaldehyde (166 μL, 2.22 mmol, 37 wt. % solution in water), glacial acetic acid (96 μL, 1.67 mmol), sodium triacetoxyborohydride (353 mg, 1.67 mmol) and stirred at room temperature for 15 hours. The reaction was diluted with dichloromethane and washed with saturated aqueous sodium bicarbonate. The organic layer was separated and the aqueous extracted with dichloromethane. The organic layers were combined, dried over magnesium sulfate, filtered, concentrated, and purified by flash chromatography (0-10% ammonium hydroxide in acetonitrile) to give 0.276 g (64% yield from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine) (8S)—N-methyl-N-{[5-(4-methyl-1-piperazinyl)imidazo[1,2-a]pyridin-2-yl]methyl}-5,6,7,8-tetrahydro-8-quinolinamine as a pale yellow oil. ¹H-NMR (CDCl₃): δ 8.52 (d, 1H), 7.70 (s, 1H), 7.34 (d, 1H), 7.28 (d, 1H), 7.10 (m, 1H), 7.06 (m, 1H), 6.23 (dd, 1H), 4.12 (m, 1H), 3.96 (s, 2H), 3.14 (m, 4H), 2.86-2.78 (m, 2H), 2.71-2.65 (m, 4H), 2.41 (s, 3H), 2.39 (s, 3H), 2.16 (m, 1H), 2.06-1.97 (m, 2H), 1.68 (m, 1H); MS m/z 391 (M+1). ¹³C-NMR (CDCl₃): δ158.0, 147.0, 146.2, 145.5, 145.2, 136.4, 134.1, 124.7, 121.4, 111.9, 107.9, 98.9, 62.5, 55.0, 55.0, 53.1, 49.5, 49.5, 46.1, 39.0, 29.2, 24.2, 21.1; HRMS: Calculated Mass: 391.2610; Found Mass: 391.2614; Formula: C₂₃H₃₁N₆. Analysis Calculated for C₂₃H₃₀N₆: C, 70.74; H, 7.74; N, 21.52. Found: C, 70.36; H, 7.77; N, 21.53.

Alternatively (8S)—N-methyl-N-{[5-(4-methyl-1-piperazinyl)imidazo[1,2-a]pyridin-2-yl]methyl}-5,6,7,8-tetrahydro-8-quinolinamine can be synthesized in the following fashion:

### A) 5-bromoimidazo[1,2-a]pyridine-2-carbaldehyde

The reactor is charged with 2-amino-6-bromopyridine (3.0 Kg, 17.3 mol) and dimethoxyethane (12 Liters) and stirred under nitrogen. 1,1,3-Trichloroacetone (5.6 Kg, 30.3 mol) is added to the 25° C. solution in a single portion and the reaction solution is warmed to 65° C. jacket temperature and maintained for approximately 2 to 4 hours until judged complete. The reaction is cooled to 10° C. and held for approximately one hour and filtered. The solids are rinsed with dimethoxyethane (6 Liters). The solid is placed back in the reactor and treated with dimethoxyethane (12 Liters) and 2N HCl (12 Liters) and warmed to approximately 75 degrees for 16 to 20 hours or until judged complete. The reaction is cooled to approximately 10° C. and pH is adjusted to approximately 8 with 3 N NaOH. The resulting solids are filtered and washed with water. The solid is dried at 50° C. for 16 hours to yield 5-bromoimidazo[1,2-a]pyridine-2-carbaldehyde, (2.81 Kg, 72% yield) ¹H NMR (400 MHz, DMSO-D6) δ ppm 10.05 (s, 1H) 8.66 (s, 1. H) 7.72 (s, 1H) 7.42 (s, 1H) 7.35 (s, 1H)

### B) 5-(4-methyl-1-piperazinyl)imidazo[1,2-a]pyridine-2-carbaldehyde

The reactor is charged with N-methylpiperazine (3.1 Kg, 31 mol) and tetrahydrofuran (10 Liters) and stirred under nitrogen while cooling to negative 20° C. n-Butyl lithium (10.4 L, 26.0 mol) is added to the reaction at a rate to maintain the negative 20° C. temp and the contents are stirred for 15 to 30 minutes. A slurry of 5-bromoimidazo[1,2-a]pyridine-2-carbaldehyde (2.79 Kg, 12.4 mol) in tetrahydrofuran (10 Liters) is added at a rate to maintain the reaction at <0° C. The slurry is washed in with additional tetrahydrofuran (6 Liters). The reaction is stirred for 30 minutes and warmed to approximately negative 10° C. The reaction is quenched by addition of 6N HCl solution to achieve pH 4.0 while maintaining at <15° C. The reaction is diluted with heptane (14 Liters) and the layers allowed to separate. The lower aqueous layer is drained and the upper organic layer is washed with 1N HCl (2×1.5 Liters). The combined aqueous layers are stirred at 20 degrees and adjusted to pH 9 with 4N NaOH solution. The Aqueous layer is extracted with 10% iPrOH/CH₂Cl₂ (3×28 Liters) and the combined organic layers are washed with saturated NaHCO3 solution (14 Liters) and evaporated at <25° C. to approximately 3 volumes. Isopropanol (28 Liters) is added and reaction again concentrated under reduced pressure to approximately 8.5 Liters. Isopropanol (17 Liters) is added and the reaction is treated with a solution of oxalic acid (1.0 Kg, 11.1 mol) in isopropanol (7 Liters) at a rate to maintain good stirring and temperature between approximately 25-40° C. The reaction is stirred for 30 minutes and the solids are collected and washed with isopropanol (8.5 Liters) Solids are dried at 50° C. to yield 5-(4-methyl-1-piperazinyl)imidazo[1,2-a]pyridine-2-carbaldehyde, (2.25 Kg, 54% yield) ¹H NMR (400 MHz, DMSO-D6) δ ppm 10.01 (s, 1H) 8.47 (s, 1H) 7.41 (m, 2H) 6.65 (m, 1H) 3.34 (s, 8H) 2.78 (s, 3H)

### C) (8S)—N-{(1S)-1-[4-(methyloxy)phenyl]ethyl}-5,6,7,8-tetrahydro-8-quinolinamine

A slurry of sodium triacetoxyborohydride (4.54 Kg, 21.4 mol.) in dichloromethane (22 Liters) is treated with 6,7-dihydro-8(5H)-quinolinone (1.8 Kg, 12.3 mol.) followed by (1S)-1-[4-(methyloxy)phenyl]ethanamine (1.8 Kg, 11.9 mol).) and the reaction was allowed to stirr vigorously at 22° C. for 24 hrs. The reaction is quenched with 1N NaOH (aprox 27 Liters) to achieve pH 8 in the aqueous layer. The phases were separated and the organic phase was treated with 1 N sodium hydroxide (aprox 3.5 Liters) to achieve pH 11 in the aqueous layer. The phases again separated. The dichloromethane solution was then concentrated to minimum volume and treated with heptane (18 Liters). The volume again concentrated to aprox 9 Liters. Precipitation occurred upon cooling to 22° C. The suspension was further cooled to 0° C. and filtered. Solids were dried at ambient temperature under vacuum with nitrogen to give (8S)—N-{(1S)-1-[4-(methyloxy)phenyl]ethyl}-5,6,7,8-tetrahydro-8-quinolinamine. (2.18 Kg, 63%) ¹H NMR (400 MHz, DMSO-D6) δ ppm 8.36 (m, 1H) 7.44 (m, 1H) 7.29 (m, 2H) 7.15 (m, 1H) 6.83 (m, 2H) 4.00 (m, 1H) 3.70 (s, 3H) 3.59-3.64 (m, 1H) 2.66 (m, 1H) 2.64 (s, 1H) 2.53 (s, 1H) 1.76 (s, 1H) 1.64 (s, 1H) 1.50 (s, 1H) 1.39 (s, 1H) 1.24 (m, 3H)

### D) (8S)—N-methyl-5,6,7,8-tetrahydro-8-quinolinamine

A slurry of sodium triacetoxyborohydride (2.44 Kg, 11.5 molo) and (8S)—N-{(1S)-1-[4-(methyloxy)phenyl]ethyl}-5,6,7,8-tetrahydro-8-quinolinamine (2.17 Kg, 7.7 mol.) in dichloromethane (21.8 Liters) is cooled to 5° C. Formaldehyde solution (37 wt. % in water, 744 ml, 10 mol.) is added slowly to maintain the temperature below 25 deg C. The solution is stirred for 30 min at 22° C. The reaction is then quenched with trifluoroacetic acid (7.3 Liters, 95 mol.) added slowly. Upon completion of the addition, the reaction is warmed up to 30° C. and stirred for 16 hrs. Water (11 Liters) is added and the two phases separated. The aqueous phase is washed with dichloromethane (14 Liters) and the combined organic phases washed with water (2×5.5 Liters). The organic phase is discarded. The pH of the aqueous phase is raised to 8.5-9 by the addition of 6N NaOH and the aqueous layer extracted with dichloromethane (3×13 Liters). The dichloromethane is exchanged for isopropanol to achieve a final volume of aprox. 75 Liters. This solution is then treated with a solution of oxalic acid (588 g, 6.5 Mol.) in isopropanol (2.2 Liters) to induce precipitation. After stirring for 2 hours, the suspension filtered at 22° C. to and the solids dried at 22° C. to afford (8S)—N-methyl-5,6,7,8-tetrahydro-8-quinolinamine oxalate salt. (1.07 Kg, 55% yield) ¹H NMR (300 MHz, DMSO-D6) δ ppm 9.25 (br s, 1H) 8.52 (s, 1H) 7.69 (s, 1H) 7.39 (s, 1H) 4.39 (s, 1H) 2.82 (s, 2H) 2.65 (s, 3H) 2.50 (s, 1H) 2.32 (s, 1H) 1.99 (s, 1H) 1.80 (s, 1H);

For (8S)—N-methyl-5,6,7,8-tetrahydro-8-quinolinamine as free base: ¹H-NMR (CDCl₃): δ 8.37 (d, 1H), 7.36 (d, 1H), 7.06 (dd, 1H), 3.65 (m, 1H), 2.76 (m, 2H), 2.53 (s, 3H), 2.11 (m, 1H), 1.97 (m, 1H), 1.75 (m, 2H); MS m/z 163 (M+1).

### E) (8S)—N-methyl-N-{[5-(4-methyl-1-piperazinyl)imidazo[1,2-a]pyridin-2-yl]methyl}-5,6, 7,8-tetrahydro-8-quinolinamine

A slurry of sodium triacetoxyborohydride (0.63 g, 2.97 mmol) and (8S)—N-methyl-5,6, 7,8-tetrahydro-8-quinolinamine. (0.5 g, 1.98 mmol.) in DCM (50 ml.) is stirred at 20° C. To this is added 5-(4-methyl-1-piperazinyl)imidazo[1,2-a]pyridine-2-carbaldehyde oxylate (0.84 g, 2.97 mmol.) and the reaction is allowed to stir at 20° C. for 16 hours. The reaction was then quenched with 2N NaOH to achieve pH 12 and the layers allowed to separate. The aqueous layer is washed with additional dichloromethane (3×10 ml) and the combined organic layers were evaporated to oil that was dried under high vacuum to give (8S)—N-methyl-N-{[5-(4-methyl-1-piperazinyl)imidazo[1,2-a]pyridin-2-yl]methyl}-5,6,7,8-tetrahydro-8-quinolinamine as a tan oil (0.6 g, 77%) ¹H NMR compares to above.

### Example 25

### [5-(4-Methyl-1-piperazinyl)-2-({methyl[(8S)-5,6,7,8-tetrahydro-8-quinolinyl]amino}methyl)imidazo[1,2-a]pyridin-3-yl]methanol

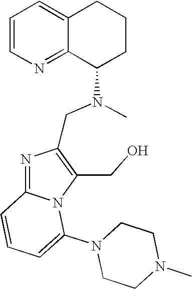

A solution of (8S)—N-methyl-N-{[5-(4-methyl-1-piperazinyl)imidazo[1,2-a]pyridin-2-yl]methyl}-5,6,7,8-tetrahydro-8-quinolinamine (2.9 g, 7.4 mmol) in formaldehyde (10 mL, 37 wt. % solution in water) and glacial acetic acid (2.5 mL) was heated at 50° C. for 15 hours. The reaction mixture was cooled, diluted with dichloromethane, and washed with saturated aqueous sodium carbonate. The organic layer was isolated and the aqueous washed three times with dichloromethane/isopropyl alcohol. The organic layers were combined, dried with magnesium sulfate, filtered, and concentrated. The residue was purified by flash chromatography (0-10% ammonium hydroxide in acetonitrile) to give 2.1 g (68% yield) [5-(4-methyl-1-piperazinyl)-2-({methyl[(8S)-5,6,7,8-tetrahydro-8-quinolinyl]amino}methyl)imidazo[1,2-a]pyridin-3-yl]methanol as a white solid. The solid was recrystallized from dichloromethane and hexanes. ¹H-NMR (CDCl₃): δ 8.42 (d, 1H), 7.31 (m, 2H), 7.06 (m, 1H), 7.01 (m, 1H), 6.75 (s, 1H), 6.39 (d, 1H), 5.29 (m, 2H), 4.01 (m, 3H), 3.52 (m, 1H), 3.38 (m, 1H), 2.90 (m, 4H), 2.78 (m, 1H), 2.67 (m, 1H), 2.52 (m, 2H), 2.40 (s, 3H), 2.21 (m, 1H), 2.13 (s, 3H), 1.96 (m, 2H), 1.68 (m, 1H); MS m/z 443 (M+Na)⁺. ¹³C-NMR (CDCl₃): δ 157.1, 148.4, 147.2, 146.2, 145.5, 136.9, 134.3, 125.2, 124.4, 121.7, 113.7, 102.0, 61.9, 55.11, 54.9, 54.0, 53.6, 51.9, 51.7, 46.3, 36.9, 29.4, 21.6, 21.5.

High Resolution MS: Calculated Mass: 421.2710; Found Mass: 421.2707; Formula: C₂₄H₃₃N₆O.

Analysis Calculated for C₂₄H₃₂N₆O: C, 68.54; H, 7.67; N, 19.98. Found: C, 68.26; H, 7.72; N, 19.89. Absolute stereochemistry confirmed by X-ray.

### Example 26

### [5-(4-methyl-1-piperazinyl)-2-({methyl[(8S)-5,6,78-tetrahydro-8-quinolinyl]amino}methyl)imidazo[1,2-a]pyridin-3-yl]methanol

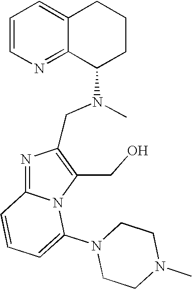

A slurry of sodium triacetoxyborohydride (1.86 Kg) and (8S)—N-methyl-5,6,7,8-tetrahydro-8-quinolinamine oxalate salt. (1.3 Kg, 5.15 mol.) in dichloromethane (13 Liters.) is stirred at 20° C. A solution of 5-(4-methyl-1-piperazinyl)imidazo[1,2-a]pyridine-2-carbaldehyde oxylate (2.07 Kg, 6.18 mol.) and triethylamine (1.25 Kg, 12.4 mol.) in dichloromethane (6.5 Liters) is added to the reaction at a rate to maintain the temperature below 30° C. The reaction is stirred at 20° C. for 16 hours. The reaction was then quenched with 2N NaOH to achieve pH 12 (Aprox 13 Liters). Methanol (aprox 6 Liters) is added to achieve a bilayer. The lower organic layer is separated and aqueous layer washed with dichloromethane (4×5 Liters). The combined organic layers were evaporated to minimum stir volume and the solvent was exchanged for water to achieve a final concentration of 6.5 Liters. This solution was maintained at 40° C. and treated with 37% aqueous formaldehyde solution (2.7 Liters, 35 mol.). Solution allowed to stir at 40° C. for 24 hours and additional formaldehyde solution added (1.35 Liters, 18 mol). Reaction was allowed to stir for 72 hours and then cooled to 25° C. and treated with saturated aqueous sodium bicarbonate (5.2 Liters vol) and dichloromethane (6.5 Liters). The layers separated and the aqueous layer washed with additional dichloromethane (2×6.5 Liters). The combined organic layer washed with sodium bicarbonate solution (4 Liters) and then the organic layer filtered through a bed of silica gel 60 (3.9 Kg). The silica bed washed with additional dichloromethane (3×6.5 Liters) and the combined organic solvent was evaporated to minimum stir volume. Ethyl acetate (13 Liters) was added and the solvent again evaporated to a final concentration of 6.5 Liters. The solution was cooled slowly and crystallization occurred. The solids are filtered and rinsed with ethyl acetate (2.6 Liters). Solids dried at 45° C. to give [5-(4-methyl-1-piperazinyl)-2-({methyl[(8S)-5,6,7,8-tetrahydro-8-quinolinyl]amino}methyl)imidazo[1,2-a]pyridin-3-yl]methanol. (1.15 Kg, 53%) ¹H NMR (400 MHz, DMSO-D6) δ ppm 7.47 (s, 1H) 7.23 (s, 1H) 7.12 (s, 2H) 6.53 (s, 1H) 5.95 (s, 1H) 5.09 (s, 2H) 3.89 (s, 3H) 3.30 (s, 4H) 2.77 (s, 5H) 2.64 (s, 1H) 2.47 (s, 1H) 2.27 (s, 5H) 2.01 (s, 4H) 1.89 (s, 2H) 1.58 (s, 1H)

### Example 27

### N,N,N′-Trimethyl-N′-[2-({methyl[(8S)-5,6,7,8-tetrahydro-8-quinolinyl]amino}methyl)imidazo[1,2-a]pyridin-5-yl]-1,2-ethanediamine (Intermediate)

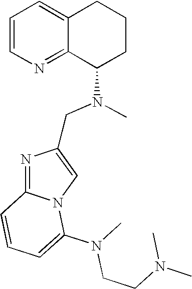

N,N,N′-Trimethyl-N′-[2-({methyl[(8S)-5,6,7,8-tetrahydro-8-quinolinyl]amino}methyl)imidazo[1,2-a]pyridin-5-yl]-1,2-ethanediamine was prepared from (8S)—N-[(5-fluoroimidazo[1,2-a]pyridin-2-yl)methyl]-N-methyl-5,6,7,8-tetrahydro-8-quinolinamine and N,N,N′-trimethylethylene diamine via thermal displacement in a similar manner as described herein to give a yellow oil (64% yield). ¹H NMR (400 MHz, CDCl₃) δ 8.46 (d, J=4.4 Hz, 1H), 7.70 (s, 1H), 7.30 (d, J=7.7 Hz, 1H), 7.22 (d, J=8.9 Hz, 1H), 7.06 (dd, J=8.7, 7.3 Hz, 1H), 7.00 (dd, J=7.3, 4.8 Hz, 1H), 6.22 (d, J=7.3 Hz, 1H), 4.05 (m, 1H), 3.91 (s, 2H), 3.14 (m, 2H), 2.82 (s, 3H), 2.77 (m, 1H), 2.63 (m, 1H), 2.49 (t, J=7.2 Hz, 2H), 2.34 (s, 3H), 2.17 (s, 6H), 2.07-1.95 (m, 3H), 1.63 (m, 1H); MS m/z 393 (M+1).

### Example 28

### [5-[[2-(Dimethylamino)ethyl](methyl)amino]-2-({methyl[(8S)-5,6,7,8-tetrahydro-8-quinolinyl]amino}methyl)imidazo[1,2-a]pyridin-3-yl]methanol

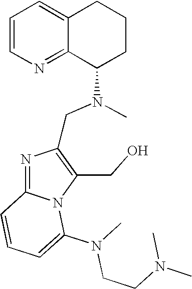

[5-[[2-(Dimethylamino)ethyl](methyl)amino]-2-({methyl[(8S)-5,6,7,8-tetrahydro-8-quinolinyl]amino}methyl)imidazo[1,2-a]pyridin-3-yl]methanol was prepared from N,N,N′-trimethyl-N′-[2-({methyl[(8S)-5,6,7,8-tetrahydro-8-quinolinyl]amino}methyl)imidazo[1,2-a]pyridin-5-yl]-1,2-ethanediamine via hydroxymethylation in a similar manner as shown herein to give a yellow solid (48% yield). ¹H NMR (400 MHz, CDCl₃) δ 8.49-8.41 (m, 1H), 7.36-7.29 (m, 2H), 7.09-6.98 (m, 2H), 6.41 (d, J=7.1 Hz, 1H), 5.15-4.89 (m, 2H), 4.06-3.80 (m, 3H), 3.29 (m, 1H), 3.09 (m, 1H), 2.82-2.75 (m, 2H), 2.71 (s, 3H), 2.49-2.41 (m, 2H), 2.32 (s, 3H), 2.16-2.13 (m, 6H), 2.03-1.96 (m, 3H), 1.67 (m, 1H); MS m/z 423 (M+1).

### Example 29

### N-Methyl-N-{[5-(4-methyl-1-piperazinyl)imidazo[1,2-a]pyridin-2-yl]methyl}-6,7,8,9-tetrahydro-5H-cyclohepta[b]pyridin-9-amine (Intermediate)

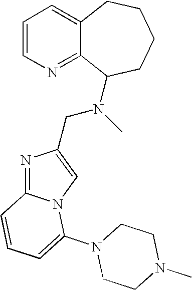

### A) 6,7,8,9-Tetrahydro-5H-cyclohepta[b]pyridin-9-ol

To a solution of 6,7,8,9-tetrahydro-5H-cyclohepta[b]pyridine (26.32 g, 179 mmol) in 100 mL of glacial acetic acid was added 30% aqueous hydrogen peroxide (36 mL) and the mixture heated at 70° C. for 16 hours. The reaction mixture was concentrated and the residue dissolved in chloroform. Solid sodium carbonate (100 g) was added and the mixture stirred for 2 hours. The solids were filtered off and the wash concentrated. The residue was dissolved in acetic anhydride (400 mL) and the mixture heated at 90° C. for 48 hours. The mixture was concentrated and the residue dissolved in water (500 mL) and potassium carbonate (50 g) was added carefully portionwise. Methanol (20 mL) was added and the mixture heated to 70° C. for 16 hours. The mixture was allowed to cool and extracted 3 times with 150 mL of dichloromethane. The organic layers were combined and concentrated. The residue was purified by silica chromatography eluting with a 1% to 2% gradient of 2M ammonia/methanol in dichloromethane. Appropriate fractions were concentrated to give 6.97 g (24%) of 6,7,8,9-tetrahydro-5H-cyclohepta[b]pyridin-9-ol. ¹H NMR (400 MHz, CDCl₃) δ 1.2 (m, 1H), 1.4 (m, 1H), 1.8 (m, 1H), 2.0 (m, 1H), 2.1 (m, 1H), 2.2 (m, 1H), 2.7 (m, 2H), 4.8 (d, J=11.2 Hz, 1H), 5.9 (s, 1H), 7.1 (dd, J=7.5, 4.9 Hz, 1H), 7.4 (d, J=7.3 Hz, 1H), 8.4 (d, J=4.8 Hz, 1H); MS m/z 164 (M+1).

### B) 5,6,7,8-Tetrahydro-9H-cyclohepta[b]pyridin-9-one

To a −78° C. solution of 2M oxalyl chloride in dichloromethane (23 mL, 46 mmol) in dichloromethane (150 mL) was added a solution of dimethyl sulfoxide (7.1 mL, 100 mmol) in dichloromethane (20 mL). The mixture was stirred for 10 minutes and a solution of 6,7,8,9-tetrahydro-5H-cyclohepta[b]pyridin-9-ol (6.9 g, 42 mmol) in 30 mL of dichloromethane added dropwise. The mixture was stirred for 30 minutes and triethylamine (21 g, 210 mmol) added dropwise. The mixture was allowed to warm to room temperature and stirred for 1 hour. The mixture washed with water, dried over magnesium sulfate, and concentrated. A solid formed while concentrating and was filtered off. The wash was concentrated and the residue purified by silica chromatography eluting with ethyl acetate. Appropriate fractions were concentrated to yield 5.12 g (74%) of 5,6,7,8-tetrahydro-9H-cyclohepta[b]pyridin-9-one. ¹H NMR (400 MHz, CDCl₃) δ 1.9 (m, 4H), 2.8 (m, 2H), 2.9 (m, 2H), 7.3 (m, 1H), 7.6 (d, 1H), 1.9 (d, 1H); MS m/z 162 (M+1).

### C)N-Methyl-6,7,8,9-tetrahydro-5H-cyclohepta[b]pyridin-9-amine

N-Methyl-6,7,8,9-tetrahydro-5H-cyclohepta[b]pyridin-9-amine was prepared from 5,6,7,8-tetrahydro-9H-cyclohepta[b]pyridin-9-one and methyl amine via reductive amination in a similar manner as described herein to give 23 mg (38% yield) of a yellow oil. ¹H NMR (400 MHz, CDCl₃) δ 8.33 (m, 1H), 7.33 (d, J=7.4 Hz, 1H), 7.00 (dd, J=7.3, 4.9 Hz, 1H), 3.75 (d, J=9.5 Hz, 1H), 2.81 (m, 1H), 2.73 (m, 1H), 2.59 (s, 1H), 2.44 (s, 3H), 1.98 (m, 2H), 1.77 (m, 2H), 1.43 (m, 2H).

### D) N-Methyl-N-{[5-(4-methyl-1-piperazinyl)imidazo[1,2-a]pyridin-2-yl]methyl}-6,7,8,9-tetrahydro-5H-cyclohepta[b]pyridin-9-amine

N-Methyl-N-{[5-(4-methyl-1-piperazinyl)imidazo[1,2-a]pyridin-2-yl]methyl}-6,7,8,9-tetrahydro-5H-cyclohepta[b]pyridin-9-amine was prepared from N-methyl-6,7,8,9-tetrahydro-5H-cyclohepta[b]pyridin-9-amine and 5-(4-methyl-1-piperazinyl)imidazo[1,2-a]pyridine-2-carbaldehyde via reductive amination in a similar manner as described herein to give 34 mg (64% yield) of a white oil. ¹H NMR (300 MHz, CDCl₃) δ 8.36 (m, 1H), 7.53 (s, 1H), 7.39-7.31 (m, 2H), 7.15 (dd, J=9.0, 7.2 Hz, 1H), 7.06 (dd, J=7.4, 4.9 Hz, 1H), 6.28 (d, J=7.0 Hz, 1H), 3.88-3.62 (m, 4H), 3.19 (s, 4H), 2.71 (s, 4H), 2.55 (m, 1H), 2.45 (s, 3H), 2.33 (s, 3H), 2.26 (m, 2H), 1.95 (m, 1H), 1.80 (m, 2H), 1.48 (m, 1H); MS m/z 405 (M+1).

### Example 30

### (5-(4-Methyl-1-piperazinyl)-2-{[methyl(6,7,8,9-tetrahydro-5H-cyclohepta[b]pyridin-9-yl)amino]methyl}imidazo[1,2-a]pyridin-3-yl)methanol

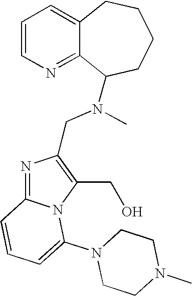

(5-(4-Methyl-1-piperazinyl)-2-{[methyl(6,7,8,9-tetrahydro-5H-cyclohepta[b]pyridin-9-yl)amino]methyl}imidazo[1,2-a]pyridin-3-yl)methanol was prepared from N-methyl-N-{[5-(4-methyl-1-piperazinyl)imidazo[1,2-a]pyridin-2-yl]methyl}-6,7,8,9-tetrahydro-5H-cyclohepta[b]pyridin-9-amine via hydroxymethylation in a similar manner as shown herein to give a white solid (85% yield). ¹H NMR (400 MHz, CDCl₃) δ 8.37 (d, J=4.6 Hz, 1H), 7.38-7.33 (m, 2H), 7.11-7.07 (m, 1H), 7.04 (dd, J=7.3, 4.9 Hz, 1H), 6.42 (d, J=7.1 Hz, 1H), 5.16 (dd, J=43.5, 13.7 Hz, 2H), 4.01 (m, 1H), 3.87 (m, 1H), 3.74 (m, 1H), 3.39-3.31 (m, 2H), 3.20 (m, 1H), 2.95-2.88 (m, 4H), 2.64 (m, 1H), 2.50-2.43 (m, 2H), 2.39 (s, 3H), 2.24 (s, 3H), 2.10 (m, 1H), 2.01-1.97 (m, 2H), 1.77-1.71 (m, 2H), 1.63 (m, 1H); MS m/z 435 (M+1).

### Example 31

### (8S)—N-Ethyl-N-{[5-(4-methyl-1-piperazinyl)imidazo[1,2-a]pyridin-2-yl]methyl}-5,6,7,8-tetrahydro-8-quinolinamine (Intermediate)

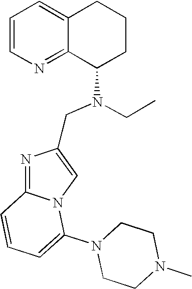

(8S)—N-Ethyl-N-{[5-(4-methyl-1-piperazinyl)imidazo[1,2-a]pyridin-2-yl]methyl}-5,6,7,8-tetrahydro-8-quinolinamine was prepared from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine and acetaldehyde via deprotection and reductive amination in a similar manner as described herein to give a pale yellow oil (19% yield, 2 steps). ¹H NMR (400 MHz, CDCl₃) δ 8.47 (d, J=4.4 Hz, 1H), 7.65 (s, 1H), 7.29 (d, J=7.7 Hz, 1H), 7.24-7.23 (m, 1H), 7.10-7.06 (m, 1H), 7.00 (dd, J=7.6, 4.7 Hz, 1H), 6.21 (d, J=7.2 Hz, 1H), 4.21 (m, 1H), 4.01-3.83 (m, 2H), 3.12 (s, 4H), 2.90-2.82 (m, 2H), 2.79-2.72 (m, 2H), 2.66 (s, 4H), 2.40 (s, 3H), 2.14 (m, 1H), 2.02-1.88 (m, 2H), 1.64 (m, 1H), 1.07 (t, J=7.0 Hz, 3H); MS m/z 405 (M+1).

### Example 32

### [2-({Ethyl[(8S)-5,6,7,8-tetrahydro-8-quinolinyl]amino}methyl)-5-(4-methyl-1-piperazinyl)imidazo[1,2-a]pyridin-3-yl]methanol

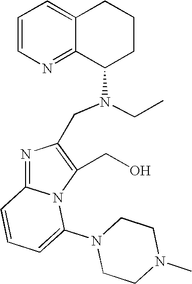

A solution of [2-({{(1S)-1-[4-(methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol (166 mg, 0.31 mmol) in dichloromethane (1 mL) was treated with trifluoroacetic acid (0.5 mL) and stirred at room temperature for 1.25 hours. The reaction was concentrated, diluted with dichloromethane, and washed with saturated aqueous sodium carbonate. The organic layer was separated, concentrated, and purified by flash chromatography (0-10% ammonium hydroxide in acetonitrile) to give 31 mg (25% yield) of the deprotected intermediate (5-(4-methyl-1-piperazinyl)-2-{[(8S)-5,6,7,8-tetrahydro-8-quinolinylamino]methyl}imidazo[1,2-a]pyridin-3-yl)methanol. This intermediate (30 mg, 0.074 mmol) was dissolved in dichloroethane (750 μL) and treated with acetaldehyde (8.3 μL, 0.15 mmol), glacial acetic acid (6.3 μL, 0.11 mmol), and sodium triacetoxyborohydride (24 mg, 0.11 mmol) and stirred at room temperature for 15 hours. The reaction was diluted with dichloromethane and washed with saturated aqueous sodium carbonate. The organic layer was separated and the aqueous extracted with dichloromethane. The organic layers were combined, concentrated, and purified by preparative chromatography (0-10% ammonium hydroxide in acetonitrile) to give 22 mg (69% yield) [2-({ethyl[(8S)-5,6,7,8-tetrahydro-8-quinolinyl]amino}methyl)-5-(4-methyl-1-piperazinyl)imidazo[1,2-a]pyridin-3-yl]methanol as a white solid. ¹H NMR (400 MHz, CDCl₃) δ 8.36 (d, J=4.4 Hz, 1H), 7.31-7.26 (m, 2H), 7.06 (m, 1H), 6.98 (dd, J=7.5, 4.9 Hz, 1H), 6.40 (d, J=7.2 Hz, 1H), 5.52 (d, J=13.2 Hz, 1H), 5.20 (d, J=12.9 Hz, 1H), 4.19 (d, J=14.0 Hz, 1H), 3.89-3.85 (m, 2H), 3.75 (m, 1H), 3.18 (m, 1H), 3.00-2.88 (m, 3H), 2.80-2.71 (m, 2H), 2.65-2.56 (m, 2H), 2.51-2.45 (m, 2H), 2.40 (s, 3H), 2.38-2.33 (m, 2H), 1.97 (m, 1H), 1.79 (m, 1H), 1.59 (m, 1H), 1.13 (t, J=7.1 Hz, 3H); MS m/z 435 (M+1).

Alternatively, [2-({Ethyl[(8S)-5,6,7,8-tetrahydro-8-quinolinyl]amino}methyl)-5-(4-methyl-1-piperazinyl)imidazo[1,2-a]pyridin-3-yl]methanol may be made from a compound of Example 31, (8S)—N-Ethyl-N-{[5-(4-methyl-1-piperazinyl)imidazo[1,2-a]pyridin-2-yl]methyl}-5,6,7,8-tetrahydro-8-quinolinamine by hydroxymethylation.

### Example 33

### (8S)—N-(1-Methylethyl)-N-{[5-(4-methyl-1-piperazinyl)imidazo[1,2-a]pyridin-2-yl]methyl}-5,6,7,8-tetrahydro-8-quinolinamine (Intermediate)

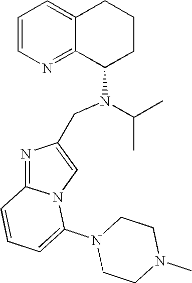

(8S)—N-(1-Methylethyl)-N-{[5-(4-methyl-1-piperazinyl)imidazo[1,2-a]pyridin-2-yl]methyl}-5,6,7,8-tetrahydro-8-quinolinamine was prepared from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine and acetone via deprotection and reductive amination in a similar manner as described herein to give a pale yellow oil (50% yield, 2 steps). ¹H NMR (400 MHz, CDCl₃) δ 8.44 (d, J=4.5 Hz, 1H), 7.64 (s, 1H), 7.24-7.18 (m, 2H), 7.06-7.02 (m, 1H), 6.96 (dd, J=7.6, 4.7 Hz, 1H), 6.17 (d, J=7.1 Hz, 1H), 4.22 (m, 1H), 3.92 (dd, J=29.9, 16.6 Hz, 2H), 3.20-3.14 (m, 1H), 3.10 (s, 4H), 2.81-2.73 (m, 2H), 2.66 (s, 4H), 2.41 (s, 3H), 2.03-1.93 (m, 3H), 1.63 (m, 1H), 1.12 (m, 6H); MS m/z 419 (M+1).

### Example 34

### [2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol

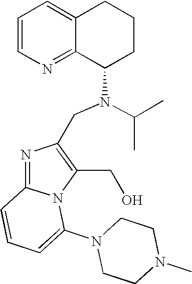

[2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol was prepared from [2-({{(1S)-1-[4-(methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol and acetone via deprotection and reductive amination in a similar manner as shown herein to give a white solid (39% yield). ¹H NMR (400 MHz, CDCl₃) δ 8.31 (s, 1H), 7.31-7.25 (m, 2H), 7.05 (m, 1H), 6.96 (m, 1H), 6.40 (d, J=7.1 Hz, 1H), 5.55 (d, J=13.0 Hz, 1H), 5.14-5.11 (m, 1H), 4.20-4.16 (m, 1H), 4.00-3.97 (m, 1H), 3.83-3.79 (m, 2H), 3.12 (m, 1H), 3.01 (m, 1H), 2.93-2.88 (m, 2H), 2.81-2.70 (m, 3H), 2.65-2.58 (m, 2H), 2.51-2.44 (m, 2H), 2.41 (s, 3H), 1.97 (m, 1H), 1.82 (m, 1H), 1.57 (m, 1H), 1.19 (m, 6H); MS m/z 449 (M+1).

Alternatively, [2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol may be formed from (8S)—N-(1-Methylethyl)-N-{[5-(4-methyl-1-piperazinyl)imidazo[1,2-a]pyridin-2-yl]methyl}-5,6,7,8-tetrahydro-8-quinolinamine by hydroxymethylation.

### Example 35

### (8S)—N-{[5-(4-Methyl-1-piperazinyl)imidazo[1,2-a]pyridin-2-yl]methyl}-N-propyl-5,6,7,8-tetrahydro-8-quinolinamine (Intermediate)

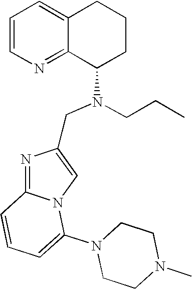

(8S)—N-{[5-(4-Methyl-1-piperazinyl)imidazo[1,2-a]pyridin-2-yl]methyl}-N-propyl-5,6,7,8-tetrahydro-8-quinolinamine was prepared from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine and propionaldehyde via deprotection and reductive amination in a similar manner as described herein to give a pale yellow oil (40% yield, 2 steps). ¹H NMR (400 MHz, CDCl₃) δ 8.45 (d, J=4.4 Hz, 1H), 7.67 (s, 1H), 7.28 (d, J=7.6 Hz, 1H), 7.24-7.22 (m, 1H), 7.08-7.04 (m, 1H), 6.99 (dd, J=7.6, 4.7 Hz, 1H), 6.20 (d, J=7.2 Hz, 1H), 4.16 (m, 1H), 3.94 (dd, J=62.0, 15.2 Hz, 2H), 3.12 (s, 4H), 2.77-2.60 (m, 8H), 2.39 (s, 3H), 2.11 (m, 1H), 1.99-1.86 (m, 2H), 1.47 (m, 2H), 1.62 (m, 1H), 0.81 (t, J=7.3 Hz, 3H); MS m/z 419 (M+1).

### Example 36

### [5-(4-Methyl-1-piperazinyl)-2-({propyl[(8S)-5,6,7,8-tetrahydro-8-quinolinyl]amino}methyl)imidazo[1,2-a]pyridin-3-yl]methanol

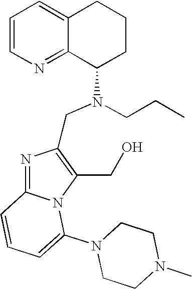

[5-(4-Methyl-1-piperazinyl)-2-({propyl[(8S)-5,6,7,8-tetrahydro-8-quinolinyl]amino}methyl)imidazo[1,2-a]pyridin-3-yl]methanol was prepared from [2-({{(1S)-1-[4-(methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol and propionaldehyde via deprotection and reductive amination in a similar manner as shown herein to give a white solid (64% yield). ¹H NMR (400 MHz, CDCl₃) δ 8.36 (d, J=4.5 Hz, 1H), 7.32-7.26 (m, 2H), 7.06 (dd, J=8.7, 7.2 Hz, 1H), 6.98 (dd, J=7.7, 4.7 Hz, 1H), 6.41 (d, J=7.1 Hz, 1H), 5.52 (d, J=12.9 Hz, 1H), 5.17 (d, J=13.0 Hz, 1H), 4.20 (d, J=13.4 Hz, 1H), 3.90 (d, J=13.5 Hz, 1H), 3.85 (m, 1H), 3.77 (m, 1H), 3.17 (m, 1H), 2.98 (m, 1H), 2.91 (m, 2H), 2.77 (m, 2H), 2.62 (m, 2H), 2.49 (m, 1H), 2.41 (s, 3H), 2.35 (m, 2H), 2.20 (m, 1H), 1.98 (m, 1H), 1.81 (m, 1H), 1.64-1.56 (m, 3H), 0.78 (t, J=7.3 Hz, 3H); MS m/z 449 (M+1).

Alternatively, [5-(4-Methyl-1-piperazinyl)-2-({propyl[(8S)-5,6,7,8-tetrahydro-8-quinolinyl]amino}methyl)imidazo[1,2-a]pyridin-3-yl]methanol may be formed from (8S)—N-{[5-(4-Methyl-1-piperazinyl)imidazo[1,2-a]pyridin-2-yl]methyl}-N-propyl-5,6,7,8-tetrahydro-8-quinolinamine by hydroxymethylation.

### Example 37

### (8S)—N-(Cyclopropylmethyl)-N-{[5-(4-methyl-1-piperazinyl)imidazo[1,2-a]pyridin-2-yl]methyl}-5,6,7,8-tetrahydro-8-quinolinamine (Intermediate)

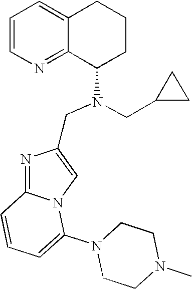

(8S)—N-(Cyclopropylmethyl)-N-{[5-(4-methyl-1-piperazinyl)imidazo[1,2-a]pyridin-2-yl]methyl}-5,6,7,8-tetrahydro-8-quinolinamine was prepared from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine and cyclopropane carboxaldehyde via deprotection and reductive amination in a similar manner as described herein to give a pale yellow oil (14% yield, 2 steps). ¹H NMR (400 MHz, CDCl₃) δ 8.47 (d, J=4.5 Hz, 1H), 7.72 (s, 1H), 7.30-7.26 (m, 2H), 7.08 (dd, J=8.9, 7.2 Hz, 1H), 7.00 (dd, J=7.5, 4.7 Hz, 1H), 6.22 (d, J=7.2 Hz, 1H), 4.36 (m, 1H), 4.17-3.94 (m, 2H), 3.13 (s, 4H), 2.81-2.74 (m, 2H), 2.67 (s, 4H), 2.57-2.51 (m, 2H), 2.40 (s, 3H), 2.18 (m, 1H), 2.00-1.88 (m, 2H), 1.65 (m, 1H), 0.95 (m, 1H), 0.44-0.37 (m, 2H), 0.11-0.05 (m, 2H); MS m/z 431 (M+1).

### Example 38

### [2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol

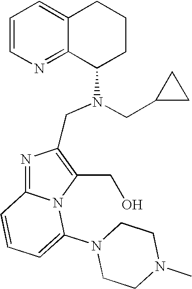 [2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol was prepared from [2-({{(1S)-1-[4-(methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol and cyclopropane carboxaldehyde via deprotection and reductive amination in a similar manner as shown herein to give a white solid (69% yield). ¹H NMR (400 MHz, CDCl₃) δ 8.37 (d, J=4.6 Hz, 1H), 7.33-7.26 (m, 2H), 7.07 (dd, J=8.7, 7.3 Hz, 1H), 6.99 (dd, J=7.5, 4.7 Hz, 1H), 6.42 (d, J=6.9 Hz, 1H), 5.54 (d, J=13.0 Hz, 1H), 5.31 (d, J=13.1 Hz, 1H), 4.47 (d, J=14.0 Hz, 1H), 3.92-3.89 (m, 2H), 3.78 (m, 1H), 3.18 (m, 1H), 3.01-2.89 (m, 3H), 2.83-2.71 (m, 2H), 2.64-2.58 (m, 2H), 2.50 (m, 1H), 2.42 (s, 3H), 2.36-2.29 (m, 2H), 2.16 (m, 1H), 1.99-1.86 (m, 2H), 1.76 (m, 1H), 1.59 (m, 1H), 1.06 (m, 1H), 0.58 (m, 1H), 0.39 (m, 1H), 0.11 (m, 1H), 0.03 (m, 1H); MS m/z 461 (M+1).

Alternatively, [2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol may be formed from (8S)—N-(Cyclopropylmethyl)-N-{[5-(4-methyl-1-piperazinyl)imidazo[1,2-a]pyridin-2-yl]methyl}-5,6,7,8-tetrahydro-8-quinolinamine by hydroxymethylation.

### Example 39

### (8S)—N-{[5-(4-Methyl-1-piperazinyl)imidazo[1,2-a]pyridin-2-yl]methyl}-N-(phenylmethyl)-5,6,7,8-tetrahydro-8-quinolinamine (Intermediate)

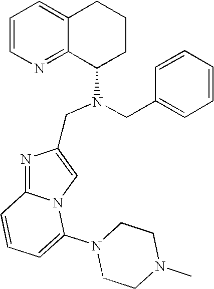

(8S)—N-{[5-(4-Methyl-1-piperazinyl)imidazo[1,2-a]pyridin-2-yl]methyl}-N-(phenylmethyl)-5,6,7,8-tetrahydro-8-quinolinamine was prepared from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine and benzaldehyde via deprotection and reductive amination in a similar manner as described herein to give an orange oil (88% yield, 2 steps). ¹H NMR (400 MHz, CDCl₃) δ 8.51 (d, J=4.4 Hz, 1H), 7.73 (s, 1H), 7.52-7.50 (m, 2H), 7.28-7.22 (m, 4H), 7.14 (m, 1H), 7.08-7.04 (m, 1H), 7.00 (dd, J=7.7, 4.7 Hz, 1H), 6.18 (d, J=7.4 Hz, 1H), 4.16 (m, 1H), 4.11-3.77 (m, 4H), 3.10 (s, 4H), 2.80-2.72 (m, 2H), 2.66 (s, 4H), 2.42 (s, 3H), 2.15 (m, 1H), 2.00-1.91 (m, 2H), 1.62 (m, 1H); MS m/z 467 (M+1).

### Example 40

### [5-(4-Methyl-1-piperazinyl)-2-([(phenylmethyl)[(8S)-5,6,7,8-tetrahydro-8-quinolinyl]amino]methyl)imidazo[1,2-a]pyridin-3-yl]methanol

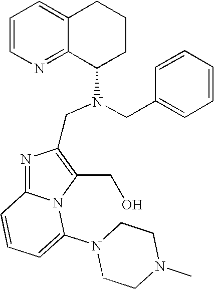

[5-(4-Methyl-1-piperazinyl)-2-({(phenylmethyl)[(8S)-5,6,7,8-tetrahydro-8-quinolinyl]amino}methyl)imidazo[1,2-a]pyridin-3-yl]methanol was prepared from (8S)—N-{[5-(4-methyl-1-piperazinyl)imidazo[1,2-a]pyridin-2-yl]methyl}-N-(phenylmethyl)-5,6,7,8-tetrahydro-8-quinolinamine via hydroxymethylation in a similar manner as shown herein to give an off-white solid (42% yield). ¹H NMR (400 MHz, CDCl₃) δ 8.51 (d, J=4.5 Hz, 1H), 7.53 (d, J=7.3 Hz, 2H), 7.37-7.29 (m, 3H), 7.27-7.23 (m, 1H), 7.07-7.01 (m, 3H), 6.41 (d, J=7.2 Hz, 1H), 5.33 (d, J=12.2 Hz, 1H), 4.75 (d, J=13.2 Hz, 1H), 4.02 (d, J=13.4 Hz, 1H), 3.90 (m, 1H), 3.82 (d, J=13.2 Hz, 1H), 3.74 (m, 1H), 3.56 (d, J=13.0 Hz, 1H), 3.34 (d, J=12.4 Hz, 1H), 3.03 (m, 1H), 2.95-2.90 (m, 2H), 2.87-2.75 (m, 3H), 2.66-2.57 (m, 2H), 2.48-2.44 (m, 2H), 2.41 (s, 3H), 2.02-1.94 (m, 2H), 1.61 (m, 1H); MS m/z 497 (M+1).

### Example 41

### (8S)—N-{[5-(4-Methyl-1-piperazinyl)imidazo[1,2-a]pyridin-2-yl]-methyl}-N-{[4-(trifluoromethyl)phenyl]methyl}-5,6,7,8-tetrahydro-8-quinolinamine (Intermediate)

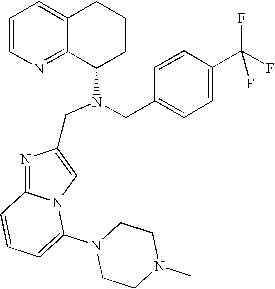

(8S)—N-{[5-(4-Methyl-1-piperazinyl)imidazo[1,2-a]pyridin-2-yl]methyl}-N-{[4-(trifluoromethyl)phenyl]methyl}-5,6,7,8-tetrahydro-8-quinolinamine was prepared from (8S)—N-{(1S)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine and 4-(trifluoromethyl)benzaldehyde via deprotection and reductive amination in a similar manner as described herein to give an orange oil (86% yield, 2 steps). ¹H NMR (400 MHz, CDCl₃) δ 8.52-8.46 (m, 1H), 7.65-7.59 (m, 3H), 7.47-7.40 (m, 2H), 7.31-7.19 (m, 2H), 7.10-6.98 (m, 2H), 6.21-6.15 (m, 1H), 4.18-4.07 (m, 2H), 4.00-3.94 (m, 2H), 3.81 (m, 1H), 3.07 (s, 4H), 2.80-2.72 (m, 2H), 2.63 (s, 4H), 2.42-2.37 (m, 3H), 2.18 (m, 1H), 2.01-1.92 (m, 2H), 1.64 (m, 1H); MS m/z 535 (M+1).

### Example 42

### {5-(4-Methyl-1-piperazinyl)-2-[((8S)-5,6,7,8-tetrahydro-8-quinolinyl{[4-(trifluoromethyl)phenyl]methyl}amino)methyl]imidazo[1,2-a]pyridin-3-yl}methanol

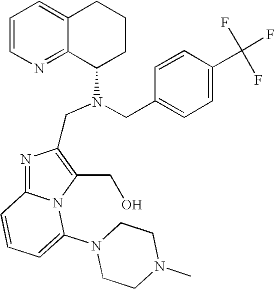

{5-(4-Methyl-1-piperazinyl)-2-[((8S)-5,6,7,8-tetrahydro-8-quinolinyl{[4-(trifluoromethyl)phenyl]methyl}amino)methyl]imidazo[1,2-a]pyridin-3-yl}methanol was prepared from (8S)—N-{[5-(4-methyl-1-piperazinyl)imidazo[1,2-a]pyridin-2-yl]methyl}-N-{[4-(trifluoromethyl)phenyl]methyl}-5,6,7,8-tetrahydro-8-quinolinamine via hydroxymethylation in a similar manner as shown herein to give an off-white solid (85% yield). ¹H NMR (400 MHz, CDCl₃) δ 8.50 (d, J=4.7 Hz, 1H), 7.65 (d, J=7.8 Hz, 2H), 7.58 (d, J=7.9 Hz, 2H), 7.32-7.28 (m, 2H), 7.07-7.01 (m, 2H), 6.87 (s, 1H), 6.40 (d, J=7.2 Hz, 1H), 5.35 (m, 1H), 4.69 (d, J=13.0 Hz, 1H), 3.96-3.83 (m, 3H), 3.71 (d, J=10.8 Hz, 1H), 3.59 (d, J=13.0 Hz, 1H), 3.42 (d, J=13.4 Hz, 1H), 3.03 (m, 1H), 2.92 (d, J=11.3 Hz, 2H), 2.86-2.73 (m, 3H), 2.66-2.54 (m, 2H), 2.47-2.43 (m, 2H), 2.41 (s, 3H), 2.01-1.90 (m, 2H), 1.60 (m, 1H); MS m/z 565 (M+1).

### Example 43

### (8S)—N-{[5-(Hexahydropyrrolo[1,2-a]pyrazin-2(1H)-yl)imidazo[1,2-a]pyridin-2-yl]methyl}-N-methyl-5,6,7,8-tetrahydro-8-quinolinamine (Intermediate)

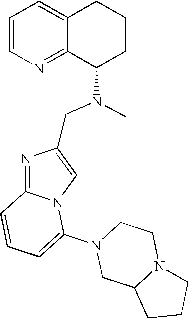

(8S)—N-{[5-(Hexahydropyrrolo[1,2-a]pyrazin-2(1H)-yl)imidazo[1,2-a]pyridin-2-yl]methyl}-N-methyl-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and 1,4-diazabicyclo[4.3.0]nonane via thermal displacement in a similar manner as described herein to give a yellow oil (82% yield). ¹H NMR (400 MHz, CDCl₃) δ 8.47 (s, 1H), 7.66 (d, J=4.8 Hz, 1H), 7.29 (d, J=7.6 Hz, 1H), 7.23 (d, J=8.9 Hz, 1H), 7.07-7.03 (m, 1H), 7.01-6.98 (m, 1H), 6.21 (d, J=7.0 Hz, 1H), 4.08 (m, 1H), 3.92 (s, 2H), 3.47 (d, J=10.8 Hz, 1H), 3.38 (d, J=11.9 Hz, 1H), 3.13-3.05 (m, 2H), 2.92-2.73 (m, 2H), 2.66-2.48 (m, 3H), 2.34 (s, 3H), 2.28-2.20 (m, 2H), 2.09 (m, 1H), 2.00-1.92 (m, 2H), 1.86-1.60 (m, 4H), 1.41 (m, 1H); MS m/z 417 (M+1).

### Example 44

### [5-(Hexahydropyrrolo[1,2-a]pyrazin-2(1H)-yl)-2-({methyl[(8S)-5,6,7,8-tetrahydro-8-quinolinyl]amino}methyl)imidazo[1,2-a]pyridin-3-yl]methanol

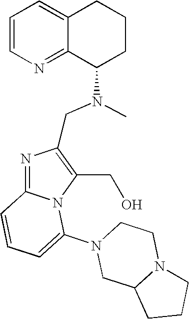

[5-(Hexahydropyrrolo[1,2-a]pyrazin-2(1H)-yl)-2-({methyl[(8S)-5,6,7,8-tetrahydro-8-quinolinyl]amino}methyl)imidazo[1,2-a]pyridin-3-yl]methanol was prepared from (8S)—N-{[5-(hexahydropyrrolo[1,2-a]pyrazin-2(1H)-yl)imidazo[1,2-a]pyridin-2-yl]methyl}-N-methyl-5,6,7,8-tetrahydro-8-quinolinamine via hydroxymethylation in a similar manner as shown herein to give a yellow solid (38% yield). ¹H NMR (400 MHz, CDCl₃) δ 8.42 (d, J=4.4 Hz, 1H), 7.32 (m, 2H), 7.07 (m, 1H), 7.02 (dd, J=7.5, 4.7 Hz, 1H), 6.43 (d, J=7.2 Hz, 1H), 5.36-5.20 (m, 2H), 4.08-3.95 (m, 3H), 3.70-3.41 (m, 2H), 3.18-3.13 (m, 2H), 2.90 (m, 1H), 2.79 (m, 1H), 2.69-2.62 (m, 2H), 2.58 (m, 1H), 2.44 (m, 1H), 2.30 (m, 1H), 2.21 (m, 1H), 2.15 (s, 3H), 2.03-1.98 (m, 2H), 1.94-1.87 (m, 2H), 1.83-1.65 (m, 2H), 1.46 (m, 1H); MS m/z 447 (M+1).

### Example 45

### (8S)—N-([5-[(3R)-3-(Dimethylamino)-1-pyrrolidinyl]imidazo[1,2-a]pyridin-2-yl]methyl)-N-methyl-5,6,7,8-tetrahydro-8-quinolinamine (Intermediate)

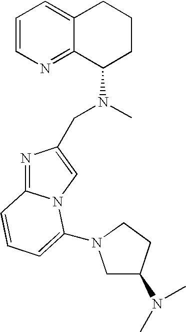

(8S)—N-({5-[(3R)-3-(Dimethylamino)-1-pyrrolidinyl]imidazo[1,2-a]pyridin-2-yl}methyl)-N-methyl-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and (3R)-(+)-3-dimethylaminopyrrolidine via thermal displacement in a similar manner as described herein to give a yellow oil (73% yield). ¹H NMR (400 MHz, CDCl₃) δ 8.46 (d, J=4.4 Hz, 1H), 7.76 (s, 1H), 7.30 (d, J=7.6 Hz, 1H), 7.13 (d, J=8.8 Hz, 1H), 7.05-6.98 (m, 2H), 6.07 (d, J=7.3 Hz, 1H), 4.10 (m, 1H), 3.97 (m, 2H), 3.54 (m, 1H), 3.42 (m, 1H), 3.30-3.22 (m, 2H), 2.87-2.73 (m, 2H), 2.62 (m, 1H), 2.35 (s, 3H), 2.26 (m, 6H), 2.17-2.10 (m, 2H), 1.98-1.86 (m, 3H), 1.62 (m, 1H); MS m/z 405 (M+1).

### Example 46

### [5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol

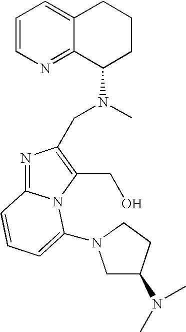

[5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol was prepared from (8S)—N-({5-[(3R)-3-(dimethylamino)-1-pyrrolidinyl]imidazo[1,2-a]pyridin-2-yl}methyl)-N-methyl-5,6,7,8-tetrahydro-8-quinolinamine via hydroxymethylation in a similar manner as shown herein to give a tan solid (28% yield). ¹H NMR (400 MHz, CDCl₃) δ 8.40 (d, J=4.2 Hz, 1H), 7.32 (d, J=7.6 Hz, 1H), 7.27 (d, J=8.9 Hz, 1H), 7.06-6.99 (m, 2H), 6.42 (d, J=7.0 Hz, 1H), 5.16 (m, 2H), 4.07-3.95 (m, 3H), 3.69 (br, 1H), 3.43 (br, 2H), 3.17 (br, 1H), 3.04-2.97 (m, 2H), 2.77 (m, 1H), 2.65 (m, 1H), 2.28 (s, 6H), 2.24-2.20 (m, 2H), 2.15 (s, 2H), 2.02-1.89 (m, 3H), 1.67 (m, 1H); MS m/z 435 (M+1).

### Example 47

### (8S)—N-{[5-(Hexahydro-1H-1,4-diazepin-1-yl)imidazo[1,2-a]pyridin-2-yl]methyl}-N-methyl-5,6,7,8-tetrahydro-8-quinolinamine (Intermediate)

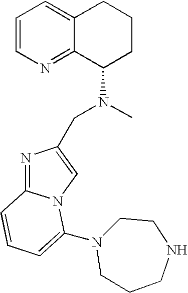

(8S)—N-{[5-(Hexahydro-1H-1,4-diazepin-1-yl)imidazo[1,2-a]pyridin-2-yl]methyl}-N-methyl-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and homopiperazine via thermal displacement in a similar manner as described herein to give a yellow oil (70% yield). ¹H NMR (400 MHz, CDCl₃) δ 8.46 (d, J=4.2 Hz, 1H), 7.64 (s, 1H), 7.29 (d, J=7.4 Hz, 1H), 7.20 (d, J=8.9 Hz, 1H), 7.06-7.02 (m, 1H), 6.99 (dd, J=7.5, 4.7 Hz, 1H), 6.24 (d, J=7.3 Hz, 1H), 4.07 (m, 1H), 3.89 (s, 2H), 3.30-3.24 (m, 4H), 3.08-3.03 (m, 4H), 2.77 (m, 1H), 2.62 (m, 1H), 2.33 (s, 3H), 2.07 (m, 1H), 1.99-1.90 (m, 4H), 1.63 (m, 1H); MS m/z 391 (M+1).

### Example 48

### [5-(Hexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol

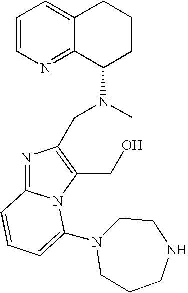

[5-(Hexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol was prepared from (8S)—N-{[5-(hexahydro-1H-1,4-diazepin-1-yl)imidazo[1,2-a]pyridin-2-yl]methyl}-N-methyl-5,6,7,8-tetrahydro-8-quinolinamine via hydroxymethylation in a similar manner as shown herein to give a clear oil (13% yield). ¹H NMR (400 MHz, CDCl₃) δ 8.43 (s, 1H), 7.34-7.31 (m, 2H), 7.10-7.01 (m, 2H), 6.51 (d, J=7.0 Hz, 1H), 5.34-5.22 (m, 2H), 4.08-3.97 (m, 3H), 3.62-3.43 (m, 2H), 3.25-3.16 (m, 3H), 3.08-3.02 (m, 3H), 2.78 (m, 1H), 2.67 (m, 1H), 2.17-2.14 (m, 5H), 2.05-1.89 (m, 3H), 1.68 (m, 1H); MS m/z 421 (M+1).

### Example 49

### (8S)—N-Methyl-N-{[5-(4-methylhexahydro-1H-1,4-diazepin-1-yl)imidazo[1,2-a]pyridin-2-yl]methyl}-5,6,7,8-tetrahydro-8-quinolinamine (Intermediate)

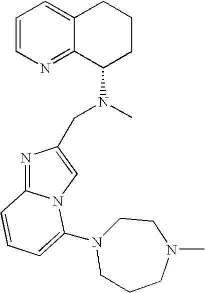

(8S)—N-Methyl-N-{[5-(4-methylhexahydro-1H-1,4-diazepin-1-yl)imidazo[1,2-a]pyridin-2-yl]methyl}-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and 1-methyl homopiperazine via thermal displacement in a similar manner as described herein to give a yellow oil (71% yield). ¹H NMR (400 MHz, CDCl₃) δ 8.49 (d, J=4.1 Hz, 1H), 7.64 (s, 1H), 7.32 (d, J=7.5 Hz, 1H), 7.21 (d, J=8.9 Hz, 1H), 7.08-7.00 (m, 2H), 6.23 (d, J=7.1 Hz, 1H), 4.09 (m, 1H), 3.92 (s, 2H), 3.38-3.34 (m, 4H), 2.85-2.63 (m, 6H), 2.42 (s, 3H), 2.36 (s, 3H), 2.10 (m, 1H), 2.03-1.95 (m, 4H), 1.66 (m, 1H); MS m/z 405 (M+1).

### Example 50

### [5-(4-Methylhexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol

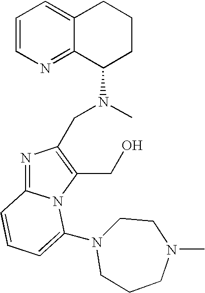

[5-(4-Methylhexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol was prepared from (8S)—N-methyl-N-{[5-(4-methylhexahydro-1H-1,4-diazepin-1-yl)imidazo[1,2-a]pyridin-2-yl]methyl}-5,6,7,8-tetrahydro-8-quinolinamine via hydroxymethylation in a similar manner as shown herein to give a clear oil (31% yield). ¹H NMR (400 MHz, CDCl₃) δ 8.42 (s, 1H), 7.34-7.29 (m, 2H), 7.08-7.00 (m, 2H), 6.49 (d, J=6.9 Hz, 1H), 5.32-5.21 (m, 2H), 4.08-3.96 (m, 3H), 3.63-3.43 (m, 2H), 3.33-3.17 (m, 2H), 2.87-2.64 (m, 6H), 2.45 (s, 3H), 2.23-2.18 (m, 2H), 2.15 (s, 3H), 2.04-1.91 (m, 3H), 1.67 (m, 1H); MS m/z 435 (M+1).

### Example 51

### (8S)—N-Methyl-N-([5-[methyl(1-methyl-3-pyrrolidinyl)amino]imidazo[1,2-a]pyridin-2-yl]methyl)-5,6,7,8-tetrahydro-8-quinolinamine (Intermediate)

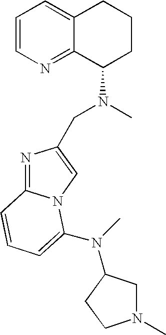

(8S)—N-Methyl-N-({5-[methyl(1-methyl-3-pyrrolidinyl)amino]imidazo[1,2-a]pyridin-2-yl}methyl)-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and N,N′-dimethyl-3-aminopyrrolidine via thermal displacement in a similar manner as described herein to give an orange oil (54% yield). ¹H NMR (400 MHz, CDCl₃) δ 8.47 (d, J=4.4 Hz, 1H), 7.67 (d, J=4.8 Hz, 1H), 7.31 (d, J=7.7 Hz, 1H), 7.24 (d, J=8.7 Hz, 1H), 7.06 (dd, J=8.7, 7.3 Hz, 1H), 7.01 (dd, J=7.6, 4.7 Hz, 1H), 6.22 (d, J=7.2 Hz, 1H), 4.09 (m, 1H), 3.97 (m, 1H), 3.90 (d, J=5.0 Hz, 2H), 2.78 (m, 2H), 2.71 (s, 3H), 2.63-2.48 (m, 4H), 2.33 (s, 3H), 2.30 (d, J=3.2 Hz, 3H), 2.13-1.95 (m, 4H), 1.84 (m, 1H), 1.66 (m, 1H); MS m/z 405 (M+1).

### Example 52

### [5-[Methyl(1-methyl-3-pyrrolidinyl)amino]-2-({methyl[(8S)-5,6,7,8-tetrahydro-8-quinolinyl]amino}methyl)imidazo[1,2-a]pyridin-3-yl]methanol

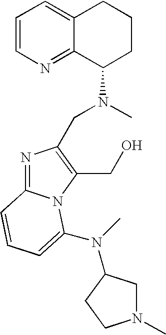

[5-[Methyl(1-methyl-3-pyrrolidinyl)amino]-2-({methyl[(8S)-5,6,7,8-tetrahydro-8-quinolinyl]amino}methyl)imidazo[1,2-a]pyridin-3-yl]methanol was prepared from (8S)—N-methyl-N-({5-[methyl(1-methyl-3-pyrrolidinyl)amino]imidazo[1,2-a]pyridin-2-yl}methyl)-5,6,7,8-tetrahydro-8-quinolinamine via hydroxymethylation in a similar manner as shown herein to give a yellow oil (52% yield). ¹H NMR (400 MHz, DMSO-d₆) δ 8.35 (d, J=4.5 Hz, 1H), 7.48 (d, J=7.6 Hz, 1H), 7.27 (d, J=8.9 Hz, 1H), 7.16-7.13 (m, 2H), 6.65-6.59 (m, 1H), 5.74 (br, 1H), 5.08-5.01 (m, 1H), 4.93-4.84 (m, 1H), 3.92-3.79 (m, 6H), 2.79-2.71 (m, 2H), 2.64-2.61 (m, 2H), 2.48 (s, 6H), 2.37-2.23 (m, 2H), 2.06 (s, 3H), 1.99-1.90 (m, 4H); MS m/z 457 (M+Na)⁺.

### Example 53

### (8S)—N-{[5-(4-Ethyl-1-piperazinyl)imidazo[1,2-a]pyridin-2-yl]methyl}-N-methyl-5,6,7,8-tetrahydro-8-quinolinamine (Intermediate)

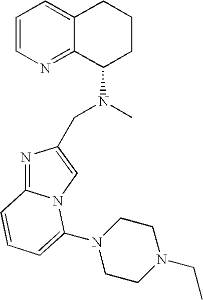

(8S)—N-{[5-(4-Ethyl-1-piperazinyl)imidazo[1,2-a]pyridin-2-yl]methyl}-N-methyl-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and N-ethylpiperazine via thermal displacement in a similar manner as described herein to give a yellow oil (87% yield). ¹H NMR (400 MHz, CDCl₃) δ 8.49 (d, J=4.4 Hz, 1H), 7.67 (s, 1H), 7.30 (d, J=7.6 Hz, 1H), 7.26-7.24 (m, 1H), 7.07 (m, 1H), 7.01 (m, 1H), 6.20 (d, J=7.2 Hz, 1H), 4.09 (m, 1H), 3.93 (s, 2H), 3.11 (s, 4H), 2.78 (m, 2H), 2.66 (s, 4H), 2.51 (q, J=7.2 Hz, 2H), 2.35 (s, 3H), 2.11 (m, 1H), 2.00-1.92 (m, 2H), 1.64 (m, 1H), 1.12 (t, J=7.0 Hz, 3H); MS m/z 405 (M+1).

### Example 54

### [5-(4-Ethyl-1-piperazinyl)-2-({methyl[(8S)-5,6,78-tetrahydro-8-quinolinyl]amino}methyl)imidazo[1,2-a]pyridin-3-yl]methanol

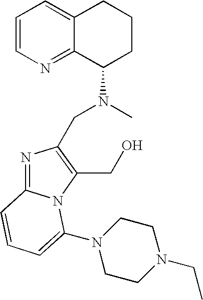

[5-(4-Ethyl-1-piperazinyl)-2-({methyl[(8S)-5,6,7,8-tetrahydro-8-quinolinyl]amino}methyl)imidazo[1,2-a]pyridin-3-yl]methanol was prepared from (8S)—N-{[5-(4-ethyl-1-piperazinyl)imidazo[1,2-a]pyridin-2-yl]methyl}-N-methyl-5,6,7,8-tetrahydro-8-quinolinamine via hydroxymethylation in a similar manner as shown herein to give an off-white solid (55% yield). ¹H NMR (400 MHz, CDCl₃) δ 8.42 (d, J=4.6 Hz, 1H), 7.33-7.29 (m, 2H), 7.08-7.04 (m, 1H), 7.01 (dd, J=7.6, 4.6 Hz, 1H), 6.40 (d, J=7.4 Hz, 1H), 5.28 (s, 2H), 4.08-3.95 (m, 3H), 3.53 (m, 1H), 3.40 (m, 1H), 3.02 (d, J=11.6 Hz, 2H), 2.94-2.87 (m, 2H), 2.77 (m, 1H), 2.66 (m, 1H), 2.56-2.43 (m, 4H), 2.21 (m, 1H), 2.13 (s, 3H), 2.02-1.89 (m, 2H), 1.67 (m, 1H), 1.14 (t, J=7.2 Hz, 3H); MS m/z 435 (M+1).

### Example 55

### (8S)—N-Methyl-N-({5-[4-(1-methylethyl)-1-piperazinyl]imidazo[1,2-a]pyridin-2-yl}methyl)-5,6,7,8-tetrahydro-8-quinolinamine (Intermediate)

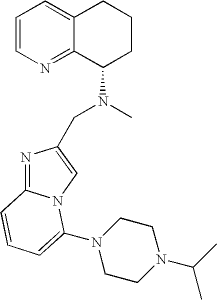

(8S)—N-Methyl-N-({5-[4-(1-methylethyl)-1-piperazinyl]imidazo[1,2-a]pyridin-2-yl}methyl)-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and 1-isopropylpiperazine via thermal displacement in a similar manner as described herein to give an off-white solid (76% yield). ¹H NMR (400 MHz, CDCl₃) δ 8.49 (d, J=4.5 Hz, 1H), 7.68 (s, 1H), 7.30 (d, J=7.6 Hz, 1H), 7.25-7.23 (m, 1H), 7.07 (m, 1H), 7.00 (m, 1H), 6.19 (d, J=7.0 Hz, 1H), 4.10 (m, 1H), 3.94 (s, 2H), 3.10 (s, 4H), 2.79-2.62 (m, 7H), 2.34 (s, 3H), 2.11 (m, 1H), 2.01-1.91 (m, 2H), 1.64 (m, 1H), 1.09 (d, J=6.7 Hz, 6H); MS m/z 419 (M+1).

### Example 56

### [5-[4-(1-Methylethyl)-1-piperazinyl]-2-({methyl[(8S)-5,6,7,8-tetrahydro-8-quinolinyl]amino}methyl)imidazo[1,2-a]pyridin-3-yl]methanol

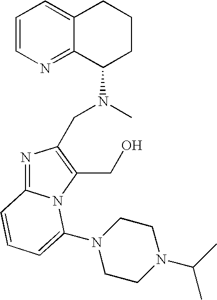

[5-[4-(1-Methylethyl)-1-piperazinyl]-2-({methyl[(8S)-5,6,7,8-tetrahydro-8-quinolinyl]amino}methyl)imidazo[1,2-a]pyridin-3-yl]methanol was prepared from (8S)—N-methyl-N-({5-[4-(1-methylethyl)-1-piperazinyl]imidazo[1,2-a]pyridin-2-yl}methyl)-5,6,7,8-tetrahydro-8-quinolinamine via hydroxymethylation in a similar manner as shown herein to give an off-white solid (50% yield). ¹H NMR (400 MHz, CDCl₃) δ 8.43 (d, J=4.5 Hz, 1H), 7.34-7.29 (m, 2H), 7.07 (m, 1H), 7.02 (dd, J=7.6, 4.7 Hz, 1H), 6.41 (d, J=7.2 Hz, 1H), 5.29 (s, 2H), 4.08-3.96 (m, 3H), 3.54 (m, 1H), 3.41 (m, 1H), 2.98 (d, J=11.2 Hz, 2H), 2.93-2.86 (m, 2H), 2.80-2.74 (m, 2H), 2.69-2.62 (m, 3H), 2.21 (m, 1H), 2.13 (s, 3H), 2.02-1.88 (m, 2H), 1.68 (m, 1H), 1.13 (d, J=6.4 Hz, 6H); MS m/z 449 (M+1).

### Example 57

### (8S)—N-([5-[(3S)-3-(Dimethylamino)-1-pyrrolidinyl]imidazo[1,2-a]pyridin-2-yl]methyl)-N-methyl-5,6,7,8-tetrahydro-8-quinolinamine (Intermediate)

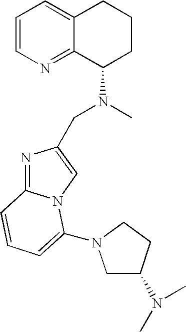

(8S)—N-({5-[(3S)-3-(Dimethylamino)-1-pyrrolidinyl]imidazo[1,2-a]pyridin-2-yl}methyl)-N-methyl-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and (3S)-(−)-3-dimethylaminopyrrolidine via thermal displacement in a similar manner as described herein to give a red oil (81% yield). ¹H NMR (400 MHz, CDCl₃) δ 8.47 (d, J=4.5 Hz, 1H), 7.72 (s, 1H), 7.30 (d, J=7.7 Hz, 1H), 7.15 (d, J=8.7 Hz, 1H), 7.06-6.99 (m, 2H), 6.08 (d, J=7.2 Hz, 1H), 4.09 (m, 1H), 3.93 (s, 2H), 3.55 (m, 1H), 3.43 (m, 1H), 3.29-3.23 (m, 2H), 2.87-2.75 (m, 2H), 2.64 (m, 1H), 2.34 (s, 3H), 2.25 (s, 6H), 2.19-2.05 (m, 2H), 1.98-1.86 (m, 3H), 1.64 (m, 1H); MS m/z 405 (M+1).

### Example 58

### [5-[(3S)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol

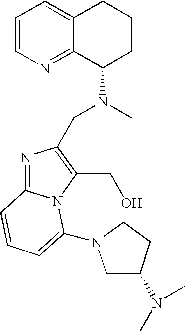

[5-[(3S)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol was prepared from (8S)—N-({5-[(3S)-3-(dimethylamino)-1-pyrrolidinyl]imidazo[1,2-a]pyridin-2-yl}methyl)-N-methyl-5,6,7,8-tetrahydro-8-quinolinamine via hydroxymethylation in a similar manner as shown herein to give a tan solid (47% yield). ¹H NMR (400 MHz, CDCl₃) δ 8.38 (d, J=4.3 Hz, 1H), 7.29 (d, J=7.7 Hz, 1H), 7.25 (d, J=8.4 Hz, 1H), 7.04-6.97 (m, 2H), 6.41 (d, J=7.0 Hz, 1H), 5.18-5.10 (m, 2H), 4.05-3.93 (m, 3H), 3.52 (br, 2H), 3.17 (br, 1H), 3.00-2.95 (m, 2H), 2.75 (m, 1H), 2.63 (m, 1H), 2.26 (s, 6H), 2.21-2.16 (m, 2H), 2.11 (s, 3H), 1.99-1.86 (m, 3H), 1.65 (m, 1H); MS m/z 435 (M+1).

### Example 59

### (8S)—N-([5-{(3R)-3-Amino-1-pyrrolidinyl]imidazo[1,2-a]pyridin-2-yl}methyl)-N-methyl-5,6,7,8-tetrahydro-8-quinolinamine (Intermediate)

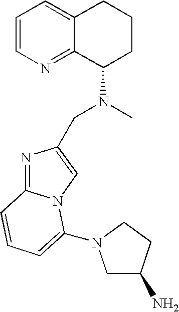

### A) 1,1-Dimethylethyl{(3R)-1-[2-({methyl[(8S)-5,6,7,8-tetrahydro-8-quinolinyl]amino}methyl)imidazo[1,2-a]pyridin-5-yl]-3-pyrrolidinyl}carbamate

1,1-Dimethylethyl{(3R)-1-[2-({methyl[(8S)-5,6,7,8-tetrahydro-8-quinolinyl]amino}methyl)imidazo[1,2-a]pyridin-5-yl]-3-pyrrolidinyl}carbamate was prepared from (8S)—N-[(5-fluoroimidazo[1,2-a]pyridin-2-yl)methyl]-N-methyl-5,6,7,8-tetrahydro-8-quinolinamine and (3R)-(+)-3-(tert-butoxycarbonyl)aminopyrrolidine via thermal displacement in a similar manner as described herein to give a tan solid (84% yield). ¹H NMR (400 MHz, CDCl₃) δ 8.52 (d, J=4.6 Hz, 1H), 7.86 (br, 1H), 7.35 (d, J=7.3 Hz, 1H), 7.21 (d, J=8.8 Hz, 1H), 7.10-7.03 (m, 2H), 6.11 (d, J=7.2 Hz, 1H), 4.94 (m, 1H), 4.39 (m, 1H), 4.16 (m, 1H), 3.98 (s, 2H), 3.70 (m, 1H), 3.52 (m, 1H), 3.24 (m, 1H), 3.10 (m, 1H), 2.82 (m, 1H), 2.68 (m, 1H), 2.42 (s, 3H), 2.34 (m, 1H), 2.14 (m, 1H), 2.04-1.94 (m, 3H), 1.67 (m, 1H), 1.47 (s, 9H); MS m/z 477 (M+1).

### B) (8S)—N-([5-[(3R)-3-Amino-1-pyrrolidinyl]imidazo[1,2-a]pyridin-2-yl]methyl)-N-methyl-5,6,7,8-tetrahydro-8-quinolinamine

(8S)—N-({5-[(3R)-3-Amino-1-pyrrolidinyl]imidazo[1,2-a]pyridin-2-yl}methyl)-N-methyl-5,6,7,8-tetrahydro-8-quinolinamine was prepared from 1,1-dimethylethyl{(3R)-1-[2-({methyl[(8S)-5,6,7,8-tetrahydro-8-quinolinyl]amino}methyl)imidazo[1,2-a]pyridin-5-yl]-3-pyrrolidinyl}carbamate via trifluoroacetic acid deprotection in a similar manner as described herein to give a pink oil (68% yield). ¹H NMR (400 MHz, CDCl₃) δ 8.48 (d, J=4.2 Hz, 1H), 7.76 (s, 1H), 7.31 (d, J=7.5 Hz, 1H), 7.14 (d, J=8.8 Hz, 1H), 7.06-6.99 (m, 2H), 6.07 (d, J=7.2 Hz, 1H), 4.09 (m, 1H), 3.91 (s, 2H), 3.71 (m, 1H), 3.57-3.49 (m, 2H), 3.32 (m, 1H), 3.10 (m, 1H), 2.78 (m, 1H), 2.64 (m, 1H), 2.34 (s, 3H), 2.26 (m, 1H), 2.08 (m, 1H), 2.01-1.92 (m, 2H), 1.76 (m, 1H), 1.64 (m, 1H); MS m/z 377 (M+1).

### Example 60

### [5-[(3R)-3-Amino-1-pyrrolidinyl]-2-({methyl[(8S)-5,6,7,8-tetrahydro-8-quinolinyl]amino}methyl)imidazo[1,2-a]pyridin-3-yl]methanol

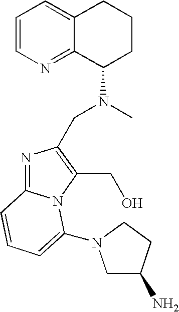

1,1-Dimethylethyl{(3R)-1-[3-(hydroxymethyl)-2-({methyl[(8S)-5,6,7,8-tetrahydro-8-quinolinyl]amino}methyl)imidazo[1,2-a]pyridin-5-yl]-3-pyrrolidinyl}carbamate was prepared from 1,1-dimethylethyl{(3R)-1-[2-({methyl[(8S)-5,6,7,8-tetrahydro-8-quinolinyl]amino}methyl)imidazo[1,2-a]pyridin-5-yl]-3-pyrrolidinyl}carbamate via hydroxymethylation in a similar manner as shown herein to give a crude red oil (50% yield). [5-[(3R)-3-Amino-1-pyrrolidinyl]-2-({methyl[(8S)-5,6,7,8-tetrahydro-8-quinolinyl]amino}methyl)imidazo[1,2-a]pyridin-3-yl]methanol was prepared from 1,1-dimethylethyl{(3R)-1-[3-(hydroxymethyl)-2-({methyl[(8S)-5,6,7,8-tetrahydro-8-quinolinyl]amino}methyl)imidazo[1,2-a]pyridin-5-yl]-3-pyrrolidinyl}carbamate via trifluoroacetic acid deprotection in a similar manner as shown herein'to give a pale yellow oil (24% yield). ¹H NMR (400 MHz, CDCl₃) δ 8.42 (d, J=4.4 Hz, 1H), 7.34-7.27 (m, 2H), 7.07-7.00 (m, 2H), 6.42 (d, J=7.1 Hz, 1H), 5.21-5.12 (m, 2H), 4.06-3.94 (m, 3H), 3.78 (m, 1H), 3.45 (br, 2H), 3.06 (br, 2H), 2.77 (m, 1H), 2.66 (m, 1H), 2.45-2.36 (m, 2H), 2.22-2.17 (m, 2H), 2.13 (s, 3H), 2.03-1.89 (m, 2H), 1.77-1.63 (m, 2H); MS m/z 407 (M+1).

### Example 61

### 1,1-Dimethylethyl methyl{(3R)-1-[2-({methyl[(8S)-5,6,7,8-tetrahydro-8-quinolinyl]amino}methyl)imidazo[1,2-a]pyridin-5-yl]-3-pyrrolidinyl}carbamate (Intermediate)

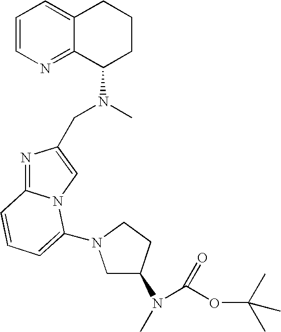

1,1-Dimethylethyl methyl{(3R)-1-[2-({methyl[(8S)-5,6,7,8-tetrahydro-8-quinolinyl]amino}methyl)imidazo[1,2-a]pyridin-5-yl]-3-pyrrolidinyl}carbamate was prepared from 1,1-dimethylethyl{(3R)-1-[2-({methyl[(8S)-5,6,7,8-tetrahydro-8-quinolinyl]amino}methyl)imidazo[1,2-a]pyridin-5-yl]-3-pyrrolidinyl}carbamate and methyl iodide via sodium hydride alkylation in a similar manner as described herein to give a yellow oil (79% yield). ¹H NMR (400 MHz, CDCl₃) δ 8.46 (d, J=4.3 Hz, 1H), 7.72 (s, 1H), 7.30 (d, J=7.5 Hz, 1H), 7.18 (d, J=8.8 Hz, 1H), 7.07-6.99 (m, 2H), 6.10 (d, J=7.3 Hz, 1H), 4.90 (br, 1H), 4.09 (m, 1H), 3.94 (s, 2H), 3.48 (m, 1H), 3.35-3.31 (m, 2H), 3.19 (m, 1H), 2.89 (s, 3H), 2.77 (m, 1H), 2.63 (m, 1H), 2.35 (s, 3H), 2.23 (m, 1H), 2.09-1.94 (m, 4H), 1.63 (m, 1H), 1.45 (s, 9H); MS m/z 491 (M+1).

### Example 62

### 1,1-Dimethylethyl{(3R)-1-[3-(hydroxymethyl)-2-({methyl[(8S)-5,6,7,8-tetrahydro-8-quinolinyl]amino}methyl)imidazo[1,2-a]pyridin-5-yl]-3-pyrrolidinyl}methylcarbamate (Intermediate)

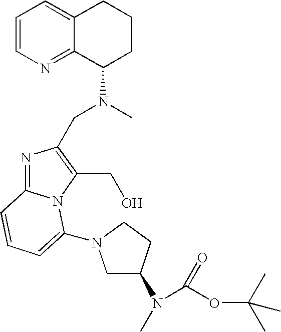

1,1-Dimethylethyl{(3R)-1-[3-(hydroxymethyl)-2-({methyl[(8S)-5,6,7,8-tetrahydro-8-quinolinyl]amino}methyl)imidazo[1,2-a]pyridin-5-yl]-3-pyrrolidinyl}methylcarbamate was prepared from 1,1-dimethylethyl methyl{(3R)-1-[2-({methyl[(8S)-5,6,7,8-tetrahydro-8-quinolinyl]amino}methyl)imidazo[1,2-a]pyridin-5-yl]-3-pyrrolidinyl}carbamate via hydroxymethylation in a similar manner as shown herein to give a yellow oil (64% yield).

¹H NMR (400 MHz, CDCl₃) δ 8.39 (d, J=4.1 Hz, 1H), 7.32-7.24 (m, 2H), 7.05-6.98 (m, 2H), 6.39 (d, J=7.0 Hz, 1H), 5.16 (s, 2H), 5.13 (br, 1H), 4.08-3.94 (m, 3H), 3.44 (br, 1H), 3.12 (br, 1H), 2.90 (s, 3H), 2.79-2.72 (m, 2H), 2.64 (m, 1H), 2.30 (m, 1H), 2.19 (m, 1H), 2.12 (s, 3H), 2.07-1.96 (m, 3H), 1.88 (m, 1H), 1.66 (m, 1H), 1.45 (s, 9H); MS m/z 521 (M+1).

### Example 63

### [5-[(3R)-3-(Methylamino)-1-pyrrolidinyl]-2-({methyl[(8S)-5,6,7,8-tetrahydro-8-quinolinyl]amino}methyl)imidazo[1,2-a]pyridin-3-yl]methanol

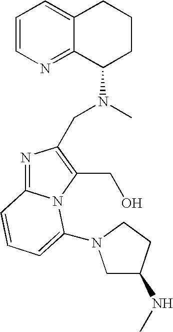

[5-[(3R)-3-(Methylamino)-1-pyrrolidinyl]-2-({methyl[(8S)-5,6,7,8-tetrahydro-8-quinolinyl]amino}methyl)imidazo[1,2-a]pyridin-3-yl]methanol was prepared from 1,1-dimethylethyl{(3R)-1-[3-(hydroxymethyl)-2-({methyl[(8S)-5,6,7,8-tetrahydro-8-quinolinyl]amino}methyl)imidazo[1,2-a]pyridin-5-yl]-3-pyrrolidinyl}methylcarbamate via trifluoroacetic acid deprotection in a similar manner as shown herein to give an orange oil (17% yield). ¹H NMR (400 MHz, CDCl₃) δ 8.42 (m, 1H), 7.33-7.27 (m, 2H), 7.06-7.00 (m, 2H), 6.43 (d, J=7.2 Hz, 1H), 5.18-5.10 (m, 2H), 4.05-3.94 (m, 3H), 3.43 (m, 3H), 3.12 (br, 2H), 2.78 (m, 1H), 2.66 (m, 1H), 2.45 (s, 3H), 2.35 (m, 1H), 2.20 (m, 1H), 2.15 (s, 3H), 2.02-1.89 (m, 2H), 1.80 (m, 1H), 1.68 (m, 1H); MS m/z 443 (M+Na)⁺.

### Example 64

### (8S)—N-({5-[(3R)-3-(Dimethylamino)-1-pyrrolidinyl]imidazo[1,2-a]pyridin-2-yl}methyl)-N-{(1S)-1-[4-(methyloxy)phenyl]ethyl}-5,6,7,8-tetrahydro-8-quinolinamine (Intermediate)

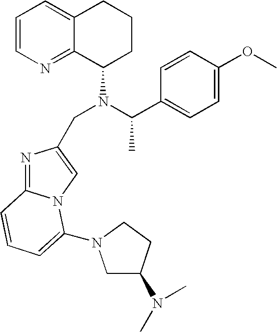

(8S)—N-({5-[(3R)-3-(Dimethylamino)-1-pyrrolidinyl]imidazo[1,2-a]pyridin-2-yl}methyl)-N-{(1S)-1-[4-(methyloxy)phenyl]ethyl}-5,6,7,8-tetrahydro-8-quinolinamine was prepared from (8S)—N-[(5-fluoroimidazo[1,2-a]pyridin-2-yl)methyl]-N-{(1S)-1-[4-(methyloxy)phenyl]ethyl}-5,6,7,8-tetrahydro-8-quinolinamine and (3R)-(+)-3-dimethylaminopyrrolidine via thermal displacement in a similar manner as described herein to give a pink oil (89% yield). ¹H NMR (400 MHz, CDCl₃) δ 8.47 (d, J=4.2 Hz, 1H), 7.91 (s, 1H), 7.55 (d, J=8.5 Hz, 2H), 7.20 (d, J=7.4 Hz, 1H), 7.08-7.00 (m, 2H), 6.95 (dd, J=7.6, 4.7 Hz, 1H), 6.82-6.80 (m, 2H), 6.05 (d, J=6.7 Hz, 1H), 4.04 (m, 1H), 4.87 (m, 1H), 3.86 (m, 2H), 3.75 (s, 3H), 3.58-3.51 (m, 2H), 3.45 (m, 1H), 3.34 (m, 1H), 2.91 (m, 1H), 2.63 (m, 1H), 2.52 (m, 1H), 2.34 (s, 6H), 2.26 (m, 1H), 2.06-1.98 (m, 2H), 1.84-1.77 (m, 2H), 1.48 (m, 1H), 1.29 (d, J=7.1 Hz, 3H); MS m/z 525 (M+1).

### Example 65

### (8S)—N-([5-[(3R)-3-(Dimethylamino)-1-pyrrolidinyl]imidazo[1,2-a]pyridin-2-yl]methyl)-N-ethyl-5,6,7,8-tetrahydro-8-quinolinamine (Intermediate)

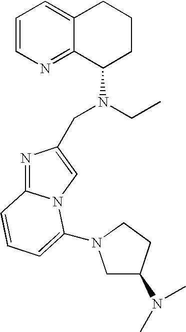

(8S)—N-({5-[(3R)-3-(Dimethylamino)-1-pyrrolidinyl]imidazo[1,2-a]pyridin-2-yl}methyl)-N-ethyl-5,6,7,8-tetrahydro-8-quinolinamine was prepared from (8S)—N-({5-[(3R)-3-(dimethylamino)-1-pyrrolidinyl]imidazo[1,2-a]pyridin-2-yl}methyl)-N-{(1S)-1-[4-(methyloxy)phenyl]ethyl}-5,6,7,8-tetrahydro-8-quinolinamine and acetaldehyde via deprotection and reductive amination in a similar manner as described herein to give a yellow oil (48% yield). ¹H NMR (400 MHz, CDCl₃) δ 8.46 (d, J=4.2 Hz, 1H), 7.72 (s, 1H), 7.29 (d, J=7.4 Hz, 1H), 7.14 (d, J=8.8 Hz, 1H), 7.06-6.98 (m, 2H), 6.09 (d, J=7.1 Hz, 1H), 4.17 (m, 1H), 3.88 (dd, J=64.6, 15.1 Hz, 2H), 3.57 (m, 1H), 3.42 (m, 1H), 3.34-3.27 (m, 2H), 2.89-2.61 (m, 5H), 2.29 (s, 6H), 2.22 (m, 1H), 2.10 (m, 1H), 1.98-1.85 (m, 3H), 1.62 (m, 1H), 1.05 (t, J=7.0 Hz, 3H); MS m/z 419 (M+1).

### Example 66

### [5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridin-3-yl]methanol

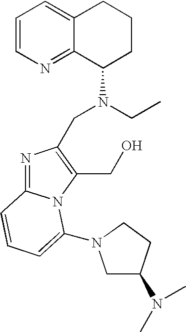

[5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridin-3-yl]methanol was prepared from (8S)—N-({5-[(3R)-3-(dimethylamino)-1-pyrrolidinyl]imidazo[1,2-a]pyridin-2-yl}methyl)-N-ethyl-5,6,7,8-tetrahydro-8-quinolinamine via hydroxymethylation in a similar manner as shown herein to give a yellow oil (28% yield). ¹H NMR (400 MHz, CDCl₃) δ 8.34 (d, J=4.4 Hz, 1H), 7.29-7.26 (m, 2H), 7.04 (m, 1H), 6.97 (dd, J=7.6, 4.7 Hz, 1H), 6.42 (d, J=7.2 Hz, 1H), 5.29-5.25 (m, 1H), 5.18-5.15 (m, 1H), 4.18 (d, J=13.2 Hz, 1H), 3.89-3.85 (m, 2H), 3.26 (br, 1H), 3.06 (m, 1H), 2.79-2.71 (m, 2H), 2.62 (m, 1H), 2.47 (m, 1H), 2.40-2.32 (m, 2H), 2.29 (s, 6H), 2.24-2.19 (m, 2H), 1.99-1.92 (m, 3H), 1.79 (m, 1H), 1.59 (m, 1H), 1.13 (t, J=7.0 Hz, 3H); MS m/z 449 (M+1).

### Example 67

### (8S)—N-([5-[(3R)-3-(Dimethylamino)-1-pyrrolidinyl]imidazo[1,2-a]pyridin-2-yl]methyl)-N-propyl-5,6,7,8-tetrahydro-8-quinolinamine (Intermediate)

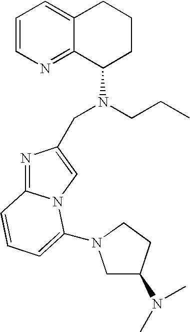

(8S)—N-({5-[(3R)-3-(Dimethylamino)-1-pyrrolidinyl]imidazo[1,2-a]pyridin-2-yl}methyl)-N-propyl-5,6,7,8-tetrahydro-8-quinolinamine was prepared from (8S)—N-({5-[(3R)-3-(dimethylamino)-1-pyrrolidinyl]imidazo[1,2-a]pyridin-2-yl}methyl)-N-{(1S)-1-[4-(methyloxy)phenyl]ethyl}-5,6,7,8-tetrahydro-8-quinolinamine and propionaldehyde via deprotection and reductive amination in a similar manner as described herein to give a yellow oil (65% yield). ¹H NMR (400 MHz, CDCl₃) δ 8.45 (d, J=4.2 Hz, 1H), 7.78 (s, 1H), 7.27 (d, J=7.4 Hz, 1H), 7.12 (d, J=8.8 Hz, 1H), 7.05-7.01 (m, 1H), 6.98 (dd, J=7.6, 4.8 Hz, 1H), 6.07 (d, J=7.3 Hz, 1H), 4.14 (m, 1H), 3.91 (dd, J=68.5, 15.3 Hz, 2H), 3.56 (m, 1H), 3.43 (m, 1H), 3.36-3.28 (m, 2H), 2.87 (m, 1H), 2.78-2.69 (m, 2H), 2.64-2.57 (m, 2H), 2.29 (s, 6H), 2.21 (m, 1H), 2.09 (m, 1H), 1.98-1.84 (m, 3H), 1.61 (m, 1H), 1.49-1.42 (m, 2H), 0.79 (t, J=7.4 Hz, 3H); MS m/z 433 (M+1).

### Example 68

### [5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridin-3-yl]methanol

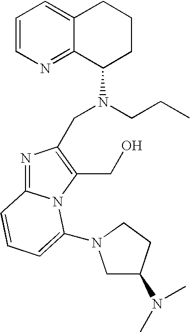

[5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridin-3-yl]methanol was prepared from (8S)—N-({5-[(3R)-3-(dimethylamino)-1-pyrrolidinyl]imidazo[1,2-a]pyridin-2-yl}methyl)-N-propyl-5,6,7,8-tetrahydro-8-quinolinamine via hydroxymethylation in a similar manner as shown herein to give a yellow oil (34% yield). ¹H NMR (400 MHz, CDCl₃) δ 8.32 (d, J=4.3 Hz, 1H), 7.28-7.26 (m, 2H), 7.04 (m, 1H), 6.96 (dd, J=7.7, 4.9 Hz, 1H), 6.43 (d, J=7.0 Hz, 1H), 5.27-5.24 (m, 1H), 5.16-5.13 (m, 1H), 4.19 (d, J=13.5 Hz, 1H), 3.90-3.82 (m, 2H), 3.24 (br, 1H), 3.06 (m, 1H), 2.79-2.70 (m, 2H), 2.60 (m, 1H), 2.43-2.32 (m, 2H), 2.28 (s, 6H), 2.22-2.15 (m, 3H), 1.98-1.91 (m, 3H), 1.80 (m, 1H), 1.63-1.54 (m, 3H), 0.77 (t, J=7.3 Hz, 3H); MS m/z 463 (M+1).

### Example 69

### (8S)—N-({5-[(3R)-3-(Dimethylamino)-1-pyrrolidinyl]imidazo[1,2-a]pyridin-2-yl}methyl)-N-(1-methylethyl)-5,6,7,8-tetrahydro-8-quinolinamine (Intermediate)

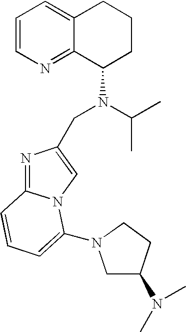

(8S)—N-({5-[(3R)-3-(Dimethylamino)-1-pyrrolidinyl]imidazo[1,2-a]pyridin-2-yl}methyl)-N-(1-methylethyl)-5,6,7,8-tetrahydro-8-quinolinamine was prepared from (8S)—N-({5-[(3R)-3-(dimethylamino)-1-pyrrolidinyl]imidazo[1,2-a]pyridin-2-yl}methyl)-N-{(1S)-1-[4-(methyloxy)phenyl]ethyl}-5,6,7,8-tetrahydro-8-quinolinamine and acetone via deprotection and reductive amination in a similar manner as described herein to give a yellow oil (49% yield). ¹H NMR (400 MHz, CDCl₃) δ 8.45 (d, J=4.3 Hz, 1H), 7.84 (s, 1H), 7.24 (m, 1H), 7.10-7.02 (m, 2H), 7.00-6.95 (m, 1H), 6.05 (d, J=7.1 Hz, 1H), 4.23 (m, 1H), 3.89 (dd, J=41.0, 16.5 Hz, 2H), 3.55 (m, 1H), 3.44-3.28 (m, 2H), 3.14 (m, 1H), 2.91-2.84 (m, 2H), 2.76 (m, 1H), 2.61 (m, 1H), 2.30 (s, 6H), 2.23 (m, 1H), 2.03 (m, 1H), 1.99-1.89 (m, 3H), 1.61 (m, 1H), 1.12-1.09 (m, 6H); MS m/z 433 (M+1).

### Example 70

### [5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol

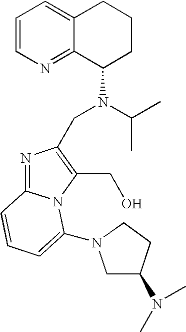

[5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol was prepared from (8S)—N-({5-[(3R)-3-(dimethylamino)-1-pyrrolidinyl]imidazo[1,2-a]pyridin-2-yl}methyl)-N-(1-methylethyl)-5,6,7,8-tetrahydro-8-quinolinamine via hydroxymethylation in a similar manner as shown herein to give a yellow oil (32% yield). ¹H NMR (400 MHz, CDCl₃) δ 8.31 (m, 1H), 7.29-7.27 (m, 2H), 7.05 (m, 1H), 6.97 (m, 1H), 6.44 (m, 1H), 5.31-5.28 (m, 1H), 5.15-5.12 (m, 1H), 4.18 (m, 1H), 4.00 (m, 1H), 3.85 (m, 1H), 3.21 (br, 1H), 3.10 (m, 1H), 2.83-2.75 (m, 3H), 2.62 (m, 1H), 2.46 (m, 1H), 2.30 (s, 6H), 2.24-2.20 (m, 2H), 2.01-1.93 (m, 3H), 1.83 (m, 1H), 1.58 (m, 1H), 1.20 (m, 6H); MS m/z 463 (M+1).

### Example 71

### (8S)—N-(Cyclopropylmethyl)-N-([5-[(3R)-3-(dimethylamino)-1-pyrrolidinyl]imidazo[1,2-a]pyridin-2-yl]methyl)-5,6,7,8-tetrahydro-8-quinolinamine (Intermediate)

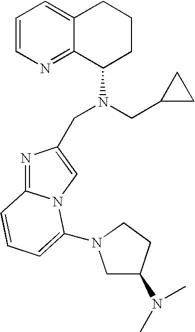

(8S)—N-(Cyclopropylmethyl)-N-({5-[(3R)-3-(dimethylamino)-1-pyrrolidinyl]imidazo[1,2-a]pyridin-2-yl}methyl)-5,6,7,8-tetrahydro-8-quinolinamine was prepared from (8S)—N-({5-[(3R)-3-(dimethylamino)-1-pyrrolidinyl]imidazo[1,2-a]pyridin-2-yl}methyl)-N-{(1S)-1-[4-(methyloxy)phenyl]ethyl}-5,6,7,8-tetrahydro-8-quinolinamine and cyclopropane carboxaldehyde via deprotection and reductive amination in a similar manner as described herein to give a yellow oil (75% yield). ¹H NMR (400 MHz, CDCl₃) δ 8.45 (d, J=4.4 Hz, 1H), 7.77 (s, 1H), 7.27 (d, J=7.8 Hz, 1H), 7.13 (d, J=8.7 Hz, 1H), 7.05-7.01 (m, 1H), 6.97 (dd, J=7.5, 4.8 Hz, 1H), 6.07 (d, J=7.3 Hz, 1H), 4.30 (m, 1H), 4.09 (d, J=15.0 Hz, 1H), 3.87 (d, J=15.0 Hz, 1H), 3.56 (m, 1H), 3.41 (m, 1H), 3.35-3.26 (m, 2H), 2.86 (m, 1H), 2.76-2.68 (m, 2H), 2.61 (m, 1H), 2.47 (m, 1H), 2.28 (s, 6H), 2.20 (m, 1H), 2.11 (m, 1H), 1.96-1.83 (m, 3H), 1.61 (m, 1H), 0.91 (m, 1H), 0.39-0.33 (m, 2H), 0.06-0.01 (m, 2H); MS m/z 445 (M+1).

### Example 72

### {2-({(Cyclopropylmethyl)[(8S)-5,6,7,8-tetrahydro-8-quinolinyl]amino}methyl)-5-[(3R)-3-(dimethylamino)-1-pyrrolidinyl]imidazo[1,2-a]pyridin-3-yl}methanol

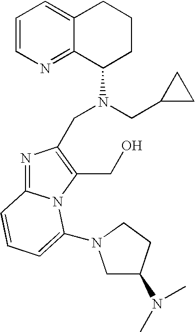

{2-({(Cyclopropylmethyl)[(8S)-5,6,7,8-tetrahydro-8-quinolinyl]amino}methyl)-5-[(3R)-3-(dimethylamino)-1-pyrrolidinyl]imidazo[1,2-a]pyridin-3-yl}methanol was prepared from (8S)—N-(cyclopropylmethyl)-N-({5-[(3R)-3-(dimethylamino)-1-pyrrolidinyl]imidazo[1,2-a]pyridin-2-yl}methyl)-5,6,7,8-tetrahydro-8-quinolinamine via hydroxymethylation in a similar manner as shown herein to give an off-white solid (35% yield). ¹H NMR (400 MHz, CDCl₃) δ 8.35 (m, 1H), 7.30-7.26 (m, 2H), 7.05 (m, 1H), 6.98 (m, 1H), 6.44 (d, J=7.0 Hz, 1H), 5.29 (s, 2H), 4.47 (d, J=13.2 Hz, 1H), 3.94-3.88 (m, 3H), 3.27 (br, 1H), 3.07 (m, 1H), 2.83-2.70 (m, 2H), 2.61 (m, 1H), 2.34-2.13 (m, 11H), 1.98-1.93 (m, 2H), 1.76 (m, 1H), 1.59 (m, 1H), 1.05 (m, 1H), 0.57 (m, 1H), 0.38 (m, 1H), 0.12-0.00 (m, 2H); MS m/z 475 (M+1).

### Example 73

### (8S)—N-Methyl-N-{1-[5-(4-methyl-1-piperazinyl)imidazo[1,2-a]pyridin-2-yl]ethyl}-5,6,7,8-tetrahydro-8-quinolinamine (Intermediate)

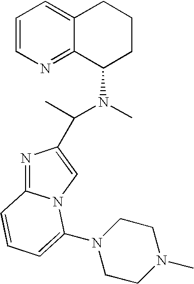

### A) 1-[5-(4-Methyl-1-piperazinyl)imidazo[1,2-a]pyridin-2-yl]ethanone

A solution of 5-(4-methyl-1-piperazinyl)imidazo[1,2-a]pyridine-2-carbaldehyde (150 mg, 0.61 mmol) in tetrahydrofuran (3 mL) at 0° C. was treated with methyl magnesium bromide (407 μL, 1.22 mmol) and stirred for 15 hours. The reaction was diluted with dichloromethane, washed with saturated aqueous sodium carbonate, separated, and concentrated to give 1-[5-(4-methyl-1-piperazinyl)imidazo[1,2-a]pyridin-2-yl]ethanol. This intermediate was dissolved in chloroform (3 mL), treated with manganese dioxide (530 mg, 6.1 mmol), and stirred for 15 hours. The reaction was filtered through celite, rinsed with dichloromethane, concentrated, and purified by flash chromatography (0-7.5% ammonium hydroxide in acetonitrile) to give 79 mg (50% yield, 2 steps) 1-[5-(4-methyl-1-piperazinyl)imidazo[1,2-a]pyridin-2-yl]ethanone. ¹H NMR (400 MHz, CDCl₃) δ 8.10 (s, 1H), 7.40 (d, J=9.1 Hz, 1H), 7.24 (m, 1H), 6.35 (d, J=7.1 Hz, 1H), 3.14 (s, 4H), 2.71 (s, 3H), 2.66 (s, 4H), 2.40 (s, 3H).

### B) (8S)—N-Methyl-N-{1-[5-(4-methyl-1-piperazinyl)imidazo[1,2-a]pyridin-2-yl]ethyl}-5,6,7,8-tetrahydro-8-quinolinamine

(8S)—N-Methyl-N-{1-[5-(4-methyl-1-piperazinyl)imidazo[1,2-a]pyridin-2-yl]ethyl}-5,6,7,8-tetrahydro-8-quinolinamine was prepared from (8S)—N-methyl-5,6,7,8-tetrahydro-8-quinolinamine and 1-[5-(4-methyl-1-piperazinyl)imidazo[1,2-a]pyridin-2-yl]ethanone via reductive amination in a similar manner as described herein to give an orange oil (47% yield). ¹H NMR (400 MHz, CD₃OD) δ 8.40 (m, 1H), 7.82 (d, J=7.0 Hz, 1H), 7.45 (d, J=7.7 Hz, 1H), 7.27-7.21 (m, 2H), 7.16-7.12 (m, 1H), 6.43 (d, J=6.5 Hz, 1H), 4.35-4.03 (m, 2H), 3.14 (s, 4H), 2.86-2.78 (m, 2H), 2.70 (s, 4H), 2.40 (d, J=2.8 Hz, 3H), 2.18-2.09 (m, 3H), 2.01-1.92 (m, 3H), 1.65 (m, 1H), 1.53 (t, J=7.0 Hz, 3H); MS m/z 405 (M+1).

### Example 74

### (8S)—N-{[5-(4-Amino-1-piperidinyl)imidazo[1,2-a]pyridin-2-yl]methyl}-N-methyl-5,6,7,8-tetrahydro-8-quinolinamine (Intermediate)

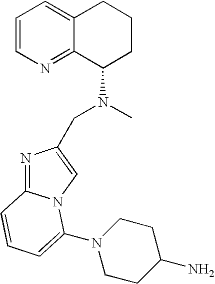

### A) (8S)—N-[(5-Fluoroimidazo[1,2-a]pyridin-2-yl)methyl]-N-[(1S)-1-[4-(methyloxy)phenyl]ethyl]-5,6,7,8-tetrahydro-8-quinolinamine

(8S)—N-[(5-Fluoroimidazo[1,2-a]pyridin-2-yl)methyl]-N-{(1S)-1-[4-(methyloxy)phenyl]ethyl}-5,6,7,8-tetrahydro-8-quinolinamine was prepared from (8S)—N-{(1S)-1-[4-(methyloxy)phenyl]ethyl}-5,6,7,8-tetrahydro-8-quinolinamine and 5-fluoroimidazo[1,2-a]pyridine-2-carbaldehyde via reductive amination in a similar manner as described herein to give a tan solid (98% yield). ¹H NMR (400 MHz, CDCl₃) δ 8.47 (d, J=4.3 Hz, 1H), 7.80 (s, 1H), 7.53 (d, J=8.4 Hz, 2H), 7.28-7.24 (m, 1H), 7.20 (d, J=7.5 Hz, 1H), 7.09 (m, 1H), 6.96 (dd, J=7.6, 4.6 Hz, 1H), 6.85 (d, J=8.6 Hz, 2H), 6.38 (dd, J=7.3, 4.8 Hz, 1H), 4.95 (m, 1H), 4.05-3.95 (m, 2H), 3.78 (s, 4H), 2.67-2.52 (m, 2H), 2.05 (m, 1H), 1.87-1.73 (m, 2H), 1.51 (m, 1H), 1.28 (d, J=6.6 Hz, 3H); MS m/z 431 (M+1).

### B) (8S)—N-[(5-Fluoroimidazo[1,2-a]pyridin-2-yl)methyl]-N-methyl-5,6,7,8-tetrahydro-8-quinolinamine

(8S)—N-[(5-Fluoroimidazo[1,2-a]pyridin-2-yl)methyl]-N-methyl-5,6,7,8-tetrahydro-8-quinolinamine was prepared from (8S)—N-[(5-fluoroimidazo[1,2-a]pyridin-2-yl)methyl]-N-{(1S)-1-[4-(methyloxy)phenyl]ethyl}-5,6,7,8-tetrahydro-8-quinolinamine and formaldehyde via deprotection and reductive amination in a similar manner as described herein to give a yellow oil (88% yield, 2 steps). ¹H NMR (400 MHz, CDCl₃) δ 8.52 (d, J=4.3 Hz, 1H), 7.79 (s, 1H), 7.36 (m, 2H), 7.13 (m, 1H), 7.05 (dd, J=7.7, 4.7 Hz, 1H), 6.39 (dd, J=7.3, 4.9 Hz, 1H), 4.07 (m, 1H), 3.92 (s, 2H), 2.76 (m, 2H), 2.43 (s, 3H), 2.02 (m, 3H), 1.70 (m, 1H); MS m/z 311 (M+1).

### C) (8S)—N-{[5-(4-Amino-1-piperidinyl)imidazo[1,2-a]pyridin-2-yl]methyl}-N-methyl-5,6,7,8-tetrahydro-8-quinolinamine

A solution of (8S)—N-[(5-fluoroimidazo[1,2-a]pyridin-2-yl)methyl]-N-methyl-5,6,7,8-tetrahydro-8-quinolinamine (94 mg, 0.30 mmol) in acetonitrile (1 mL) was treated with 4-(N—BOC-amino)piperidine (300 mg, 1.50 mmol) and heated at 50° C. for 15 hours and 70° C. for 24 hours. The reaction was diluted with dichloromethane, washed with saturated aqueous sodium carbonate, separated, concentrated, and purified by flash chromatography (0-10% ammonium hydroxide in acetonitrile) to give 117 mg (80% yield) 1,1-dimethylethyl{1-[2-({methyl[(8S)-5,6,7,8-tetrahydro-8-quinolinyl]amino}methyl)imidazo[1,2-a]pyridin-5-yl]-4-piperidinyl}carbamate as the protected intermediate. This intermediate was dissolved in dichloromethane (1 mL), treated with trifluoroacetic acid (0.50 mL,) and stirred at room temperature for 2 hours. The reaction was concentrated, diluted with dichloromethane, and washed with saturated aqueous sodium carbonate. The organic layer was isolated, concentrated, and purified by flash chromatography (0-10% ammonium hydroxide in acetonitrile) to give 72 mg (77% yield) (8S)—N-{[5-(4-amino-1-piperidinyl)imidazo[1,2-a]pyridin-2-yl]methyl}-N-methyl-5,6,7,8-tetrahydro-8-quinolinamine as a yellow oil. ¹H NMR (400 MHz, CDCl₃) δ 8.49 (d, J=3.5 Hz, 1H), 7.59 (s, 1H), 7.31 (d, J=7.7 Hz, 1H), 7.24 (d, J=8.5 Hz, 1H), 7.06 (m, 1H), 7.01 (m, 1H), 6.18 (d, J=7.4 Hz, 1H), 4.09 (m, 1H), 3.92 (s, 2H), 3.40 (d, J=11.1 Hz, 2H), 2.90 (m, 1H), 2.81-2.63 (m, 4H), 2.34 (s, 3H), 2.10 (m, 1H), 2.02-1.93 (m, 4H), 1.68-1.57 (m, 3H); MS m/z 391 (M+1).

### Example 75

### 1,1-Dimethylethyl{1-[2-({methyl[(8S)-5,6,7,8-tetrahydro-8-quinolinyl]amino}methyl)imidazo[1,2-a]pyridin-5-yl]-4-piperidinyl}carbamate (Intermediate)

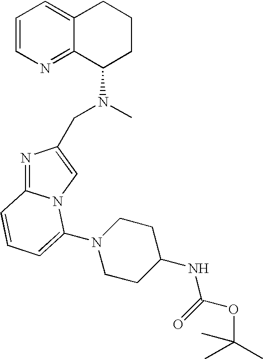

1,1-Dimethylethyl{1-[2-({methyl[(8S)-5,6,7,8-tetrahydro-8-quinolinyl]amino}methyl)imidazo[1,2-a]pyridin-5-yl]-4-piperidinyl}carbamate was prepared from (8S)—N-[(5-fluoroimidazo[1,2-a]pyridin-2-yl)methyl]-N-methyl-5,6,7,8-tetrahydro-8-quinolinamine and 4-(N—BOC-amino)piperidine via thermal displacement in a similar manner as described herein to give a tan solid (32% yield). ¹H NMR (400 MHz, CDCl₃) δ 8.47 (d, J=4.3 Hz, 1H), 7.60 (s, 1H), 7.31 (d, J=7.7 Hz, 1H), 7.25-7.23 (m, 1H), 7.08-7.04 (m, 1H), 7.01 (dd, J=7.5, 4.6 Hz, 1H), 6.18 (d, J=7.3 Hz, 1H), 4.57 (s, 1H), 4.09 (m, 1H), 3.92 (s, 2H), 3.67 (s, 1H), 3.39-3.36 (m, 2H), 2.83-2.74 (m, 3H), 2.65 (m, 1H), 2.35 (s, 3H), 2.12-1.95 (m, 5H), 1.70-1.61 (m, 3H), 1.44 (s, 9H); MS m/z 491 (M+1).

### Example 76

### [5-(4-Amino-1-piperidinyl)-2-({methyl[(8S)-5,6,7,8-tetrahydro-8-quinolinyl]amino}methyl)imidazo[1,2-a]pyridin-3-yl]methanol

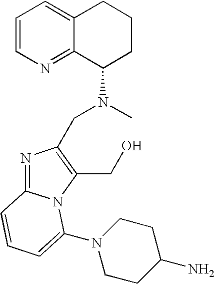

[5-(4-Amino-1-piperidinyl)-2-({methyl[(8S)-5,6,7,8-tetrahydro-8-quinolinyl]amino}methyl)imidazo[1,2-a]pyridin-3-yl]methanol was prepared from 1,1-dimethylethyl{1-[2-({methyl[(8S)-5,6,7,8-tetrahydro-8-quinolinyl]amino}methyl)imidazo[1,2-a]pyridin-5-yl]-4-piperidinyl}carbamate via hydroxymethylation (81% yield) and deprotection (48% yield) in a similar manner as shown herein to give an off-white solid. ¹H NMR (400 MHz, CDCl₃) δ 8.42 (d, J=4.3 Hz, 1H), 7.32-7.27 (m, 2H), 7.06-6.99 (m, 2H), 6.35 (d, J=7.2 Hz, 1H), 5.23 (s, 2H), 4.05-3.92 (m, 3H), 3.51 (m, 1H), 3.41 (m, 1H), 2.83-2.73 (m, 2H), 2.69-2.62 (m, 3H), 2.18 (m, 1H), 2.12 (s, 3H), 2.01-1.90 (m, 4H), 1.77-1.62 (m, 3H); MS m/z 421 (M+1).

### Example 77

### (8S)—N-[(5-{[2-(Dimethylamino)ethyl]oxy}imidazo[1,2-a]pyridin-2-yl)methyl]-N-methyl-5,6,7,8-tetrahydro-8-quinolinamine (Intermediate)

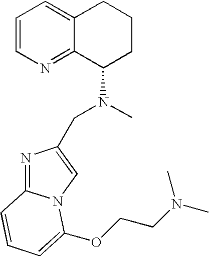

To a solution of 2-(dimethylamino)ethanol (64 μL, 0.64 mmol) in tetrahydrofuran (3.2 mL) at 0° C. was added sodium hydride (60% in oil, 43 mg, 0.64 mmol). The reaction was stirred for 30 minutes, treated with (8S)—N-[(5-fluoroimidazo[1,2-a]pyridin-2-yl)methyl]-N-methyl-5,6,7,8-tetrahydro-8-quinolinamine (100 mg, 0.32 mmol), and stirred at room temperature 15 hours. The reaction was quenched with saturated aqueous sodium carbonate, extracted into ethyl acetate, and concentrated. The residue was purified by flash chromatography (0-10% ammonium hydroxide in acetonitrile) to give 95 mg (79% yield) (8S)—N-[(5-{[2-(dimethylamino)ethyl]oxy}imidazo[1,2-a]pyridin-2-yl)methyl]-N-methyl-5,6,7,8-tetrahydro-8-quinolinamine as a yellow oil. ¹H NMR (400 MHz, CDCl₃) δ 8.45 (d, J=4.4 Hz, 1H), 7.71 (s, 1H), 7.28 (d, J=7.7 Hz, 1H), 7.13-7.11 (m, 1H), 7.06-7.02 (m, 1H), 6.98 (dd, J=7.6, 4.7 Hz, 1H), 5.93 (d, J=7.4 Hz, 1H), 4.23 (m, 2H), 4.04 (m, 1H), 3.86 (s, 2H), 2.80 (t, J=5.8 Hz, 2H), 2.74 (m, 1H), 2.62 (m, 1H), 2.33 (s, 3H), 2.32 (s, 6H), 2.06 (m, 1H), 2.00-1.89 (m, 2H), 1.62 (m, 1H); MS m/z 380 (M+1).

### Example 78

### [5-{[2-(Dimethylamino)ethyl]oxy}-2-({methyl[(8S)-5,6,7,8-tetrahydro-8-quinolinyl]amino}methyl)imidazo[1,2-a]pyridin-3-yl]methanol

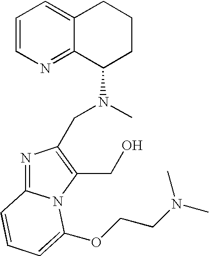 [5-{[2-(Dimethylamino)ethyl]oxy}-2-({methyl[(8S)-5,6,7,8-tetrahydro-8-quinolinyl]amino}methyl)imidazo[1,2-a]pyridin-3-yl]methanol was prepared from (8S)—N-[(5-{[2-(dimethylamino)ethyl]oxy}imidazo[1,2-a]pyridin-2-yl)methyl]-N-methyl-5,6,7,8-tetrahydro-8-quinolinamine via hydroxymethylation in a similar manner as shown herein to give a yellow oil (52% yield). ¹H NMR (400 MHz, CDCl₃) δ 8.40 (d, J=4.5 Hz, 1H), 7.28 (d, J=7.6 Hz, 1H), 7.13-7.11 (m, 1H), 7.03-6.96 (m, 2H), 5.93 (d, J=7.4 Hz, 1H), 5.08 (m, 2H), 4.24-4.21 (m, 2H), 3.96-3.92 (m, 3H), 2.89 (m, 1H), 2.83-2.71 (m, 2H), 2.61 (m, 1H), 2.31 (s, 6H), 2.18 (s, 3H), 2.11 (m, 1H), 1.98-1.89 (m, 2H), 1.61 (m, 1H); MS m/z 410 (M+1).

### Example 79

### (8S)—N-Methyl-N-[(5-{[2-(1-pyrrolidinyl)ethyl]oxy}imidazo[1,2-a]pyridin-2-yl)methyl]-5,6,7,8-tetrahydro-8-quinolinamine (Intermediate)

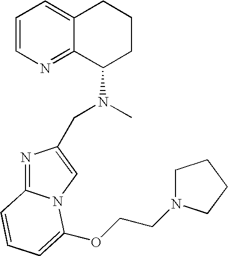

(8S)—N-Methyl-N-[(5-{[2-(1-pyrrolidinyl)ethyl]oxy}imidazo[1,2-a]pyridin-2-yl)methyl]-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and pyrrolidinoethanol via alkylation in a similar manner as described herein to give a pink oil (80% yield). ¹H NMR (400 MHz, CDCl₃) δ 8.51 (d, J=4.5 Hz, 1H), 7.77 (s, 1H), 7.33 (d, J=7.6 Hz, 1H), 7.17-7.15 (m, 1H), 7.11-7.07 (m, 1H), 7.03 (dd, J=7.6, 4.6 Hz, 1H), 5.98 (d, J=7.2 Hz, 1H), 4.33 (t, J=5.8 Hz, 2H), 4.09 (m, 1H), 3.91 (d, J=4.4 Hz, 2H), 3.01 (t, J=5.9 Hz, 2H), 2.82 (m, 1H), 2.71-2.64 (m, 4H), 2.57 (m, 1H), 2.40 (s, 3H), 2.13 (m, 1H), 2.06-1.97 (m, 2H), 1.82-1.77 (m, 4H), 1.67 (m, 1H); MS m/z 406 (M+1).

### Example 80

### (2-([Methyl[(8S)-5,6,7,8-tetrahydro-8-quinolinyl]amino]methyl)-5-{[2-(1-pyrrolidinyl)ethyl]oxy}imidazo[1,2-a]pyridin-3-yl)methanol

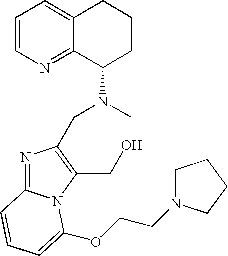

(2-({Methyl[(8S)-5,6,7,8-tetrahydro-8-quinolinyl]amino}methyl)-5-{[2-(1-pyrrolidinyl)ethyl]oxy}imidazo[1,2-a]pyridin-3-yl)methanol was prepared from (8S)—N-methyl-N-[(5-{[2-(1-pyrrolidinyl)ethyl]oxy}imidazo[1,2-a]pyridin-2-yl)methyl]-5,6,7,8-tetrahydro-8-quinolinamine via hydroxymethylation in a similar manner as shown herein to give a yellow oil (55% yield). ¹H NMR (400 MHz, CDCl₃) δ 8.42 (d, J=4.4 Hz, 1H), 7.29 (d, J=7.7 Hz, 1H), 7.13 (d, J=9.1 Hz, 1H), 7.04-6.97 (m, 2H), 5.93 (d, J=7.4 Hz, 1H), 5.08 (d, J=5.7 Hz, 2H), 4.31-4.22 (m, 2H), 3.99-3.91 (m, 3H), 3.08 (m, 1H), 2.98 (m, 1H), 2.76 (m, 1H), 2.65-2.58 (m, 5H), 2.18 (s, 3H), 2.12 (m, 1H), 1.99-1.91 (m, 2H), 1.81-1.76 (m, 4H), 1.63 (m, 1H); MS m/z 436 (M+1).

### Example 81

### (8S)—N-Methyl-N-[(5-{[2-(1-piperidinyl)ethyl]oxy}imidazo[1,2-a]pyridin-2-yl)methyl]-5,6,7,8-tetrahydro-8-quinolinamine (Intermediate)

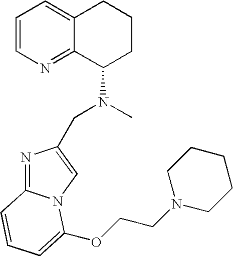

(8S)—N-Methyl-N-[(5-{[2-(1-piperidinyl)ethyl]oxy}imidazo[1,2-a]pyridin-2-yl)methyl]-5,6,7,8-tetrahydro-8-quinolinamine was prepared from (8S)—N-[(5-fluoroimidazo[1,2-a]pyridin-2-yl)methyl]-N-methyl-5,6,7,8-tetrahydro-8-quinolinamine and 1-piperidine ethanol via alkylation in a similar manner as described herein to give a yellow oil (76% yield). ¹H NMR (400 MHz, CDCl₃) δ 8.50 (d, J=4.3 Hz, 1H), 7.75 (s, 1H), 7.32 (d, J=7.6 Hz, 1H), 7.16-7.14 (m, 1H), 7.09-7.05 (m, 1H), 7.02 (dd, J=7.5, 4.7 Hz, 1H), 5.96 (d, J=7.4 Hz, 1H), 4.30 (t, J=5.9 Hz, 2H), 4.08 (m, 1H), 3.90 (s, 2H), 2.87 (t, J=5.9 Hz, 2H), 2.79 (m, 1H), 2.66 (m, 1H), 2.54-2.49 (m, 4H), 2.37 (s, 3H), 2.11 (m, 1H), 2.03-1.94 (m, 2H), 1.66 (m, 1H), 1.59-1.54 (m, 4H), 1.45-1.40 (m, 2H); MS m/z 420 (M+1).

### Example 82

### (2-({Methyl[(8S)-5,6,7,8-tetrahydro-8-quinolinyl]amino}methyl)-5-{[2-(1-piperidinyl)ethyl]oxy}imidazo[1,2-a]pyridin-3-yl)methanol

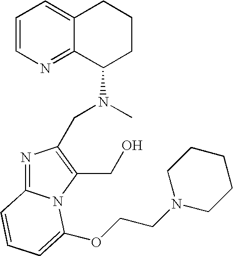

(2-({Methyl[(8S)-5,6,7,8-tetrahydro-8-quinolinyl]amino}methyl)-5-{[2-(1-piperidinyl)ethyl]oxy}imidazo[1,2-a]pyridin-3-yl)methanol was prepared from (8S)—N-methyl-N-[(5-{[2-(1-piperidinyl)ethyl]oxy}imidazo[1,2-a]pyridin-2-yl)methyl]-5,6,7,8-tetrahydro-8-quinolinamine-via hydroxymethylation in a similar manner as shown herein to give a yellow oil (67% yield). ¹H NMR (400 MHz, CDCl₃) δ 8.41 (d, J=4.3 Hz, 1H), 7.29 (d, J=7.4 Hz, 1H), 7.11 (d, J=9.0 Hz, 1H), 7.03-6.97 (m, 2H), 5.92 (d, J=7.2 Hz, 1H), 5.07 (d, J=4.2 Hz, 2H), 4.24 (t, J=5.5 Hz, 2H), 3.97 (m, 1H), 3.93 (s, 2H), 2.92-2.71 (m, 3H), 2.61 (m, 1H), 2.48 (s, 4H), 2.18 (s, 3H), 2.11 (m, 1H), 1.98-1.89 (m, 2H), 1.64-1.55 (m, 5H), 1.43-1.38 (m, 2H); MS m/z 450 (M+1).

### Example 83

### 5-(4-Methyl-1-piperazinyl)-2-({methyl[(8S)-5,6,7,8-tetrahydro-8-quinolinyl]amino}methyl)imidazo[1,2-a]pyridine-3-carbaldehyde (Intermediate)

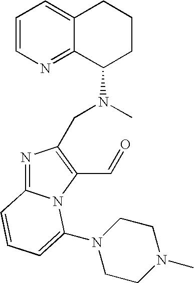

A solution of [5-(4-methyl-1-piperazinyl)-2-({methyl[(8S)-5,6,7,8-tetrahydro-8-quinolinyl]amino}methyl)imidazo[1,2-a]pyridin-3-yl]methanol (572 mg, 1.36 mmol) in dichloromethane (7 mL) was treated with IBX polystyrene (2 g, 2.8 mmol, Novabiochem), stirred at room temperature for 15 hours, treated with additional IBX polystyrene (3 g, 4.2 mmol, Novabiochem), and stirred at room temperature 24 hours. The resin was filtered, rinsed with dichloromethane, dissolved in methanol, heated at 40° C. for 15 hours, filtered, concentrated, and purified by flash chromatography (0-10% ammonium hydroxide in acetonitrile) to give 330 mg (58% yield) of 5-(4-methyl-1-piperazinyl)-2-({methyl[(8S)-5,6,7,8-tetrahydro-8-quinolinyl]amino}methyl)imidazo[1,2-a]pyridine-3-carbaldehyde as an orange oil. ¹H-NMR (CDCl₃): δ10.86 (s, 1H), 8.47 (d, 1H), 7.47 (d, 1H), 7.36 (m, 2H), 7.04 (m, 1H), 6.61 (dd, 1H), 4.29 (s, 2H), 4.23 (m, 1H), 3.35 (m, 2H), 2.94-2.81 (m, 4H), 2.71-2.67 (m, 2H), 2.50 (s, 3H), 2.40 (m, 2H), 2.37 (s, 3H), 2.19 (m, 1H), 2.08 (m, 2H), 1.71 (m, 1H).

### Example 84

### 1-[5-(4-Methyl-1-piperazinyl)-2-({methyl[(8S)-5,6,7,8-tetrahydro-8-quinolinyl]amino}methyl)imidazo[1,2-a]pyridin-3-yl]ethanol

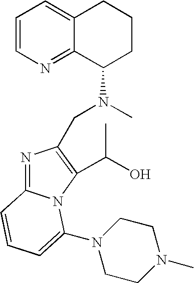

A solution of 5-(4-methyl-1-piperazinyl)-2-({methyl[(8S)-5,6,7,8-tetrahydro-8-quinolinyl]amino}methyl)imidazo[1,2-a]pyridine-3-carbaldehyde (51 mg, 0.12 mmol) in tetrahydrofuran (0.50 mL) at 0° C. was treated with methyl magnesium bromide (80 μL, 0.24 mmol), brought to room temperature, and stirred for 15 hours. The reaction was diluted with dichloromethane, washed with saturated aqueous sodium carbonate, separated, concentrated, and purified by flash chromatography (0-10% ammonium hydroxide in acetonitrile) to give 29 mg (56% yield) of 1-[5-(4-methyl-1-piperazinyl)-2-({methyl[(8S)-5,6,7,8-tetrahydro-8-quinolinyl]amino}methyl)imidazo[1,2-a]pyridin-3-yl]ethanol as a yellow solid. ¹H NMR (400 MHz, CDCl₃) δ 8.49-8.45 (m, 1H), 7.36-7.33 (m, 1H), 7.31-7.29 (m, 1H), 7.06-7.01 (m, 2H), 6.41 (d, J=6.9 Hz, 1H), 6.12-6.07 (m, 1H), 4.37 (m, 1H), 4.23-4.04 (m, 2H), 3.34 (m, 1H), 3.12-3.02 (m, 2H), 2.90-2.79 (m, 3H), 2.74-2.64 (m, 2H), 2.52 (m, 1H), 2.39-2.33 (m, 4H), 2.10-1.96 (m, 6H), 1.71 (m, 1H), 1.59-1.46 (m, 3H); MS m/z 435 (M+1).

### Example 85

### 1-[5-(4-Methyl-1-piperazinyl)-2-({methyl[(8S)-5,6,7,8-tetrahydro-8-quinolinyl]amino}methyl)imidazo[1,2-a]pyridin-3-yl]-1-propanol

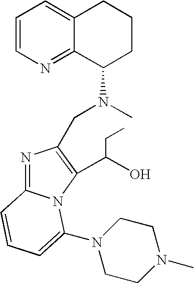

1-[5-(4-Methyl-1-piperazinyl)-2-({methyl[(8S)-5,6,7,8-tetrahydro-8-quinolinyl]amino}methyl)imidazo[1,2-a]pyridin-3-yl]-1-propanol was prepared from 5-(4-methyl-1-piperazinyl)-2-({methyl[(8S)-5,6,7,8-tetrahydro-8-quinolinyl]amino}methyl)imidazo[1,2-a]pyridine-3-carbaldehyde and ethyl magnesium bromide via a Grignard reaction in a similar manner as shown herein to give a tan solid (57% yield). ¹H NMR (400 MHz, CDCl₃) δ 8.48 (m, 1H), 7.38-7.34 (m, 1H), 7.33-7.31 (m, 1H), 7.08-7.02 (m, 2H), 6.42 (d, J=7.1 Hz, 1H), 5.82 (m, 1H), 4.45-4.00 (m, 3H), 3.43 (m, 1H), 3.20-3.11 (m, 2H), 2.94-2.83 (m, 2H), 2.75-2.54 (m, 3H), 2.39 (s, 3H), 2.37-2.34 (m, 2H), 2.14-2.00 (m, 5H), 1.93-1.83 (m, 2H), 1.74-1.65 (m, 2H), 1.00-0.84 (m, 3H); MS m/z 449 (M+1).

### Example 86

### (8S)—N-Methyl-N-{[3-[(methyloxy)methyl]-5-(4-methyl-1-piperazinyl)imidazo[1,2-a]pyridin-2-yl]methyl}-5,6,7,8-tetrahydro-8-quinolinamine

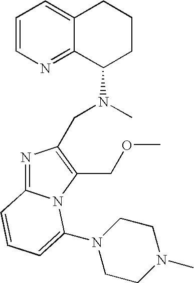

A solution of [5-(4-methyl-1-piperazinyl)-2-({methyl[(8S)-5,6,7,8-tetrahydro-8-quinolinyl]amino}methyl)imidazo[1,2-a]pyridin-3-yl]methanol (100 mg, 0.24 mmol) in tetrahydrofuran (2.4 mL) at 0° C. was treated with sodium hydride (48 mg, 0.72 mmol, 60% dispersion in oil). The reaction was stirred for 30 minutes, treated with methyl iodide (33 μL, 0.53 mmol), brought to room temperature, and stirred for 15 hours. The reaction was treated with saturated aqueous sodium carbonate, extracted into ethyl acetate, dried over magnesium sulfate, filtered, concentrated, and purified by preparative chromatography (0-60% acetonitrile-water; 0.1% trifluoroacetic acid) and then diluted with dichloromethane, washed with saturated aqueous sodium carbonate, and dried with magnesium sulfate to give 13 mg (13% yield) of an orange solid. ¹H NMR (400 MHz, CDCl₃) δ 8.32 (d, J=4.3 Hz, 1H), 7.38-7.34 (m, 2H), 7.11-7.03 (m, 2H), 6.66 (d, J=7.3 Hz, 1H), 5.18 (dd, J=28.7, 13.2 Hz, 2H), 4.10-3.83 (m, 6H), 3.78-3.72 (m, 2H), 3.56-3.52 (m, 8H), 3.28 (m, 1H), 2.81-2.63 (m, 2H), 2.17 (s, 3H), 2.02-1.86 (m, 3H), 1.66 (m, 1H); MS m/z 435 (M+1).

### Example 87

### (8S)—N-{[3-[(Ethyloxy)methyl]-5-(4-methyl-1-piperazinyl)imidazo[1,2-a]pyridin-2-yl]methyl}-N-methyl-5,6,7,8-tetrahydro-8-quinolinamine

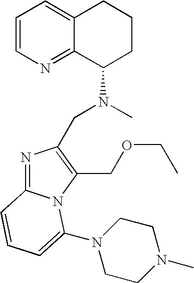

(8S)—N-{[3-[(Ethyloxy)methyl]-5-(4-methyl-1-piperazinyl)imidazo[1,2-a]pyridin-2-yl]methyl}-N-methyl-5,6,7,8-tetrahydro-8-quinolinamine was prepared from [5-(4-methyl-1-piperazinyl)-2-({methyl[(8S)-5,6,7,8-tetrahydro-8-quinolinyl]amino}methyl)imidazo[1,2-a]pyridin-3-yl]methanol and ethyl iodide via alklyation in a similar manner as shown herein to give a yellow oil (45% yield). ¹H NMR (400 MHz, CDCl₃) δ 8.53 (d, J=4.7 Hz, 1H), 7.36-7.33 (m, 2H), 7.10-7.04 (m, 2H), 6.48 (d, J=7.0 Hz, 1H), 5.05 (dd, J=42.3, 11.5 Hz, 2H), 4.05 (t, J=7.1 Hz, 1H), 3.86 (dd, J=25.0, 12.9 Hz, 2H), 3.41-3.33 (m, 2H), 3.23-3.17 (m, 2H), 2.97 (m, 1H), 2.88-2.80 (m, 4H), 2.68 (m, 1H), 2.43-2.34 (m, 8H), 2.13-2.02 (m, 3H), 1.70 (m, 1H), 1.04 (t, J=7.1 Hz, 3H); MS m/z 449 (M+1).

### Example 88

### [5-(4-Methyl-1-piperazinyl)-2-(1-{methyl[(8S)-5,6,7,8-tetrahydro-8-quinolinyl]amino}ethyl)imidazo[1,2-a]pyridin-3-yl]methanol

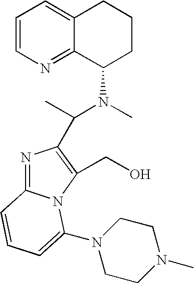 [5-(4-Methyl-1-piperazinyl)-2-(1-{methyl[(8S)-5,6,7,8-tetrahydro-8-quinolinyl]amino}ethyl)imidazo[1,2-a]pyridin-3-yl]methanol was prepared from (8S)—N-methyl-N-{1-[5-(4-methyl-1-piperazinyl)imidazo[1,2-a]pyridin-2-yl]ethyl}-5,6,7,8-tetrahydro-8-quinolinamine via hydroxymethylation in a similar manner as shown herein to give a white solid (34% yield). ¹H NMR (400 MHz, CDCl₃) δ 8.39 (m, 1H), 7.36-7.31 (m, 2H), 7.09-7.05 (m, 1H), 7.04-7.00 (m, 1H), 6.43 (dd, J=17.5, 7.1 Hz, 1H), 5.47-5.25 (m, 2H), 4.46 (m, 1H), 4.12 (m, 1H), 3.49 (m, 1H), 3.41-3.31 (m, 1H), 2.95-2.88 (m, 4H), 2.77 (m, 1H), 2.68-2.53 (m, 3H), 2.42 (s, 3H), 2.10-2.06 (m, 3H), 2.01-1.92 (m, 3H), 1.66-1.61 (m, 4H); MS m/z 435 (M+1).

### Example 89A and B

### 2,2,2-Trifluoro-1-(5-(4-methyl-1-piperazinyl)-2-{[methyl(5,6,7,8-tetrahydro-8-quinolinyl)amino]methyl}imidazo[1,2-a]pyridin-3-yl)ethanol

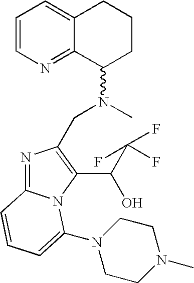

A solution of 5-(4-methyl-1-piperazinyl)-2-({methyl[(8S)-5,6,7,8-tetrahydro-8-quinolinyl]amino}methyl)imidazo[1,2-a]pyridine-3-carbaldehyde (100 mg, 0.24 mmol) in tetrahydrofuran (1.7 mL) was treated with trimethyl(trifluoromethyl)silane (85 μL, 0.57 mmol) and cooled to 0° C. Tetrabutylammonium fluoride (3.6 μL, 0.0036 mmol, 1 M in tetrahydrofuran) was added, the reaction brought to room temperature and stirred for 15 hours. Additional trimethyl(trifluoromethyl)silane (170 μL, 1.14 mmol) and tetrabutylammonium fluoride (7.2 μL, 0.0072 mmol, 1 M in tetrahydrofuran) were added and the reaction stirred for 15 hours. Tetrabutylammonium fluoride (240 μL, 0.24 mmol, 1 M in tetrahydrofuran) was added and the reaction stirred for 2 hours. The reaction was concentrated and purified by preparative chromatography (0-60% acetonitrile-water; 0.1% trifluoroacetic acid) which allowed for the separation of the two diastereomers. Each isomer was concentrated, diluted with dichloromethane, washed with saturated aqueous sodium carbonate, and dried with magnesium sulfate to give a total of 31 mg (26% yield) 2,2,2-trifluoro-1-(5-(4-methyl-1-piperazinyl)-2{[methyl(5,6,7,8-tetrahydro-8-quinolinyl)amino]methyl}imidazo[1,2-a]pyridin-3-yl)ethanol as an off-white solid.

A) First isomer off column: ¹H NMR (400 MHz, CDCl₃) δ 8.47 (d, J=4.3 Hz, 1H), 7.42 (d, J=8.8 Hz, 1H), 7.36 (d, J=7.6 Hz, 1H), 7.22-7.18 (m, 1H), 7.08-7.05 (m, 1H), 6.80 (dd, J=15.0, 7.5 Hz, 1H), 6.59 (d, J=7.1 Hz, 1H), 4.78 (br, 1H), 4.50 (br, 1H), 4.19 (br, 1H), 3.26-3.20 (m, 2H), 3.14 (m, 1H), 2.96-2.86 (m, 2H), 2.80-2.66 (m, 3H), 2.52 (m, 1H), 2.43-2.34 (m, 6H), 2.12-2.02 (m, 4H), 1.92 (m, 1H), 1.74 (m, 1H); MS m/z 489 (M+1).

B) Second isomer off column: ¹H NMR (400 MHz, CDCl₃) δ 8.50 (d, J=4.5 Hz, 1H), 7.41-7.37 (m, 2H), 7.21-7.17 (m, 1H), 7.09 (m, 1H), 6.78 (dd, J=14.7, 7.3 Hz, 1H), 6.60 (d, J=7.2 Hz, 1H), 4.36 (br, 1H), 4.16 (br, 2H), 3.30-3.21 (m, 2H), 3.11 (m, 1H), 2.95-2.86 (m, 2H), 2.83-2.70 (m, 3H), 2.55 (m, 1H), 2.42-2.32 (m, 6H), 2.04 (m, 5H), 1.74 (m, 1H); MS m/z 489 (M+1).

### Example 90

### [5-(4-Methyl-1-piperazinyl)-2-({methyl[(8R)-5,6,7,8-tetrahydro-8-quinolinyl]amino}methyl)imidazo[1,2-a]pyridin-3-yl]methanol

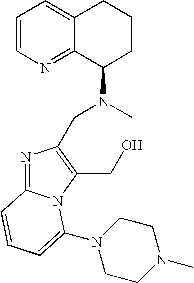

### A) 2-(Chloromethyl)-5-fluoroimidazo[1,2-a]pyridine

A solution of 6-fluoro-2-pyridinamine (6.7 g, 60 mmol) in ethyl acetate (30 mL) was treated with 1,3-dichloroacetone (15 g, 120 mmol) dissolved in ethyl acetate (15 mL) and heated at 65° C. for 15 hours. The reaction was cooled to room temperature and the precipitate filtered, rinsed with acetone and ether, and dried to yield a tan solid. This intermediate was dissolved in water and treated with saturated aqueous sodium bicarbonate until the pH=7. The precipitate was collected by filtration and dried to yield 2-(chloromethyl)-5-fluoroimidazo[1,2-a]pyridine (1.9 g, 77% yield) as a tan solid. ¹H-NMR (CDCl₃): δ 7.68 (s, 1H), 7.42 (d, 1H), 7.26-7.20 (m, 1H), 6.47 (dd, 1H), 4.76 (s, 2H).

### B) (8R)—N-{(1R)-1-[4-(Methyloxy)phenyl]ethyl}-5,6,7,8-tetrahydro-8-quinolinamine

(8R)—N-{(1R)-1-[4-(Methyloxy)phenyl]ethyl}-5,6,7,8-tetrahydro-8-quinolinamine was prepared from {(1R)-1-[4-(methyloxy)phenyl]ethyl}amine and 6,7-dihydro-8(5H)-quinolinone in a similar manner as described herein to give clear crystals (54% yield). ¹H-NMR (CDCl₃): δ 8.40 (m, 1H), 7.34 (m, 3H), 7.05 (m, 1H), 6.85 (d, 2H), 4.04 (m, 1H), 3.84-3.79 (m, 4H), 2.73-2.62 (m, 2H), 1.82 (m, 1H), 1.72 (m, 1H), 1.57 (m, 2H), 1.44 (d, 3H).

### C) (8R)—N-[(5-Fluoroimidazo[1,2-a]pyridin-2-yl)methyl]-N—{(1R)-1-[4-(methyloxy)phenyl]ethyl}-5,6,7,8-tetrahydro-8-quinolinamine

To a solution of (8R)—N-{(1R)-1-[4-(methyloxy)phenyl]ethyl}-5,6,7,8-tetrahydro-8-quinolinamine (2.32 g, 8.23 mmol) in acetonitrile (40 mL) was added N,N-diisopropylethylamine (3.15 mL, 18.1 mmol), 2-(chloromethyl)-5-fluoroimidazo[1,2-a]pyridine (1.67 g, 9.05 mmol), and potassium iodide (1.50 g, 9.05 mmol). The reaction was stirred at room temperature for 15 hours, diluted with ethyl acetate, and washed with saturated aqueous sodium bicarbonate. The organic layer was separated and the aqueous extracted with additional ethyl acetate. The organic layers were combined, dried over magnesium sulfate, filtered, concentrated, and purified by flash chromatography (0-5% ammonium hydroxide in acetonitrile) to give 2.12 g (60% yield) (8R)—N-[(5-fluoroimidazo[1,2-a]pyridin-2-yl)methyl]-N-{(1R)-1-[4-(methyloxy)phenyl]ethyl}-5,6,7,8-tetrahydro-8-quinolinamine as an off-white solid. ¹H NMR (400 MHz, CDCl₃) δ 8.47 (d, J=4.5 Hz, 1H), 7.80 (s, 1H), 7.54-7.52 (m, 2H), 7.26 (d, J=8.9 Hz, 1H), 7.20 (d, J=7.6 Hz, 1H), 7.12-7.07 (m, 1H), 6.96 (dd, J=7.6, 4.7 Hz, 1H), 6.86-6.84 (m, 2H), 6.38 (dd, J=7.4, 5.0 Hz, 1H), 4.95 (m, 1), 4.05-3.95 (m, 2H), 3.79-3.75 (m, 4H), 2.68-2.52 (m, 2H), 2.05 (m, 1H), 1.86-1.73 (m, 2H), 1.51 (m, 1H), 1.28 (d, J=6.7 Hz, 3H); MS m/z 431 (M+1).

### D) (8R)—N-{(1R)-1-[4-(Methyloxy)Phenyl]ethyl}-N-{[5-(4-methyl-1-piperazinyl)imidazo[1,2-a]pyridin-2-yl]methyl}-5,6,7,8-tetrahydro-8-quinolinamine

A solution of (8R)—N-[(5-fluoroimidazo[1,2-a]pyridin-2-yl)methyl]-N-{(1R)-1-[4-(methyloxy)phenyl]ethyl}-5,6,7,8-tetrahydro-8-quinolinamine (1.0 g, 2.32 mmol) in 1-methylpiperazine (10 mL) was heated at 100° C. for 48 hours. The reaction was diluted with dichloromethane and washed with saturated aqueous sodium bicarbonate. The organic layer was separated and the aqueous extracted with additional dichloromethane. The organic layers were combined, dried over magnesium sulfate, filtered, concentrated, and purified by flash chromatography (0-8% ammonium hydroxide in acetonitrile) to give a quantitative yield (1.2 g) of (8R)—N-{(1R)-1-[4-(methyloxy)phenyl]ethyl}-N-{[5-(4-methyl-1-piperazinyl)imidazo[1,2-a]pyridin-2-yl]methyl}-5,6,7,8-tetrahydro-8-quinolinamine as a yellow oil. ¹H NMR (400 MHz, CDCl₃) δ 8.49 (d, J=4.5 Hz, 1H), 7.78 (s, 1H), 7.62-7.60 (m, 2H), 7.24-7.19 (m, 2H), 7.08 (dd, J=8.9, 7.2 Hz, 1H), 6.98 (dd, J=7.7, 4.7 Hz, 1H), 6.86-6.84 (m, 2H), 6.21 (d, J=7.0 Hz, 1H), 4.84 (m, 1H), 4.07 (m, 1H), 3.91 (dd, J=61.8, 17.4 Hz, 2H), 3.79 (s, 3H), 3.21-3.14 (m, 4H), 2.76 (s, 4H), 2.68-2.54 (m, 2H), 2.48 (s, 3H), 2.07 (m, 1H), 1.86 (m, 2H), 1.54 (m, 1H), 1.35 (d, J=6.9 Hz, 3H); MS m/z 511 (M+1).

### E) (8R)—N-Methyl-N-{[5-(4-methyl-1-piperazinyl)imidazo[1,2-a]pyridin-2-yl]methyl}-5,6,7,8-tetrahydro-8-quinolinamine: (8R)—N-Methyl-N-{[5-(4-methyl-1-piperazinyl)imidazo[1,2-a]pyridin-2-yl]methyl}-5,6,7,8-tetrahydro-8-quinolinamine was prepared from (8R)—N-{(1R)-1-[4-(methyloxy)phenyl]ethyl}-5,6,7,8-tetrahydro-8-quinolinamine and formaldehyde via deprotection and reductive amination in a similar manner as described herein to give a pale yellow oil (53% yield, 2 steps). ¹H NMR (400 MHz, CDCl₃) δ 8.52 (d, 1H), 7.68 (s, 1H), 7.33 (d, 1H), 7.28 (d, 1H), 7.10 (dd, 1H), 7.04 (dd, 1H), 6.23 (dd, 1H), 4.12 (m, 1H), 3.96 (s, 2H), 3.13 (m, 4H), 2.85-2.77 (m, 2H), 2.70-2.65 (m, 4H), 2.39 (s, 3H), 2.37 (s, 3H), 2.14 (m, 1H), 2.05-1.96 (m, 2H), 1.68 (m, 1H); MS m/z 391 (M+1).

### F): [5-(4-Methyl-1-piperazinyl)-2-({methyl[(8R)-5,6,7,8-tetrahydro-8-quinolinyl]amino}methyl)imidazo[1,2-a]pyridin-3-yl]methanol: [5-(4-Methyl-1-piperazinyl)-2-({methyl[(8R)-5,6,7,8-tetrahydro-8-quinolinyl]amino}methyl)imidazo[1,2-a]pyridin-3-yl]methanol was prepared from (8R)—N-methyl-N-{[5-(4-methyl-1-piperazinyl)imidazo[1,2-a]pyridin-2-yl]methyl}-5,6,7,8-tetrahydro-8-quinolinamine via hydroxymethylation in a similar manner as shown herein to give a yellow oil (22% yield). ¹H-NMR (CDCl₃): δ8.42 (d, 1H), 7.31 (m, 2H), 7.06 (m, 1H), 7.02 (m, 1H), 6.40 (d, 1H), 5.29 (m, 2H), 4.01 (m, 3H), 3.52 (m, 1H), 3.38 (m, 1H), 2.90 (m, 4H), 2.78 (m, 1H), 2.67 (m, 1H), 2.52 (m, 2H), 2.40 (s, 3H), 2.21 (m, 1H), 2.14 (s, 3H), 1.96 (m, 2H), 1.69 (m, 1H); MS m/z 421 (M+1).

### Biological Section

Fusion Assay

Plasmid Generation

The complete coding sequences of HIV-1 tat (GenBank Accession No. X07861) and rev (GenBank Accession No. M34378) were cloned into pcDNA3.1 expression vectors containing G418 and hygromycin resistance genes, respectively. The complete coding sequence of the HIV-1 (HXB2 Strain) gp160 envelope gene (nucleotide bases 6225-8795 of GenBank Accession No. K03455) was cloned into plasmid pCRII-TOPO. The three HIV genes were additionally inserted into the baculovirus shuttle vector, pFastBacMam1, under the transcriptional control of the CMV promoter. A construction of the pHIV-I LTR containing mutated NFkB sequences linked to the luciferase reporter gene was prepared by digesting pcDNA3.1, containing the G418 resistance gene, with Nru I and Bam HI to remove the CMV promoter. LTR-luc was then cloned into the Nru I/Bam HI sites of the plasmid vector. Plasmid preparations were performed after the plasmids were amplified in Escherichia coli Strain DH5-alpha. The fidelity of the inserted sequences was confirmed by double-strand nucleotide sequencing using an ABI Prism Model 377 automated sequencer.

BacMam Baculovirus Generation

Recombinant BacMam baculoviruses were constructed from pFastBacMam shuttle plasmids by using the bacterial cell-based Bac-to-Bac system. Viruses were propagated in Sf9 (Spodoptera frugiperda) cells cultured in Hink's TNM-FH Insect media supplemented with 10% (v/v) fetal bovine serum and 0.1% (v/v) pluronic F-68 according to established protocols.

Cell Culture

Human osteosarcoma (HOS) cells that naturally express human CXCR4 were transfected with human CCR5, human CD4 and the pHIV-LTR-luciferase plasmid using FuGENE 6 transfection reagent. Single cells were isolated and grown under selection condition in order to generate a stable HOS (hCXCR4/hCCR5/hCD4/pHIV-LTR-luciferase) clonal cell line. The cells were maintained in Dulbeccos modified Eagles media supplemented with 10% fetal calf serum (FCS), G418 (400 ug/ml), puromycin (1 ug/ml), mycophenolic acid (40 ug/ml), xanthine (250 ug/ml) and hypoxanthine (13.5 ug/ml) to maintain a selection pressure for cells expressing the LTR-luciferase, hCCR5 and hCD4, respectively. Human embryonic kidney (HEK-293) cells stably transfected to express the human macrophage scavenging receptor (Class A, type 1; GenBank Accession No. D90187), were maintained in DMEM/F-12 media (1:1) supplemented with 10% FCS and 1.5 ug/ml puromycin. The expression of this receptor by the HEK-293 cells enhances their ability to stick to tissue culture treated plasticware.

Transduction of HEK-293 Cells

HEK-293 cells were harvested using enzyme-free cell dissociation buffer. The cells were resuspended in DMEM/F-12 media supplemented with 10% FCS and 1.5 ug/ml and counted. Tranductions were performed by direct addition of BacMam baculovirus containing insect cell media to cells. The cells were simultaneously transduced with BacMam baculovirus expressing HIV-1 tat, HIV-1 rev and HIV-1 gp160 (from the HXB2 HIV strain). Routinely an MOI of 10 of each virus was added to the media containing the cells. 2 mM butyric acid was also added to the cells at this stage to increase protein expression in transduced cells. The cells were subsequently mixed and seeded into a flask at 30 million cells per T225. The cells were incubated at 37° C., 5% CO₂, 95% humidity for 24 h to allow for protein expression.

Cell/Cell Fusion Assay Format

HEK and HOS cells were harvested in DMEM/F-12 media containing 2% FCS and DMEM media containing 2% FCS, respectively, with no selection agents added. Compounds were plated as 1 ul spots in 100% DMSO on a 96-well CulturPlate plates. HOS cells (50 ul) were added first to the wells, followed immediately by the HEK cells (50 ul). The final concentration of each cell type was 20,000 cells per well. Following these additions, the cells were returned to a tissue culture incubator (37° C.; 5% CO₂/95% air) for an additional 24 h.

Measurement of Lucifèrase Production

Following the 24 h incubation, total cellular luciferase activity was measured using the LucLite Plus assay kit (Packard, Meridien, Conn.). In brief, 100 ul of this reagent was added to each well. The plates were sealed and mixed. The plates were dark adapted for approximately 10 min prior to the luminescence being read on a Packard TopCount.

Functional Assay

Cell Culture

Human embryonic kidney (HEK-293) cells were maintained and harvested as described above. Cells were plated in 96-well, black clear bottom, poly-lysine coated plates at a concentration of 40,000 cells per well in a final volume of 100 ul containing human CXCR4BacMam (MOI=25) and Gqi5 BacMam (MOI=12.5). The cells were incubated at 37° C., 5% CO₂, 95% humidity for 24 h to allow for protein expression.

Functional FLIPR Assay

After the required incubation time the cells were washed once with 50 ul of fresh serum-free DMEM/F12 media containing probenicid. 50 ul of dye solution was then added to the cells (Calcium Plus Assay Kit Dye; Molecular Devices) was dissolved in 200 ml of the above probenicid/BSA containing media and incubated for 1 h. Cell plates were transferred to a Fluorometric Imaging Plate Reader (FLIPR). Upon addition the effect of the compounds on the change in [Ca²⁺], was examined to determine if the compounds were agonists or antagonists (ability to block SDF-1 alpha activity) at the CXCR4 receptor. IC₅₀ values are determined and pK_{b} values are calculated using the Leff and Dougall equation: K_{B}=IC₅₀/((2+([agonist]/EC₅₀ˆb)ˆ1/b−1) Where IC₅₀ is that defined by the antagonist concentration-response curve [agonist] is the EC₈₀ concentration of agonist used EC₅₀ is that defined by the agonist concentration-response curve b is the slope of the agonist concentration-response curve.

HOS HIV-1 Infectivity Assay

HIV Virus Preparation

Compounds were profiled against two HIV-1 viruses, the M-tropic (CCR5 utilizing) Ba-L strain and the T-tropic (CXCR4 utilizing) IIIB strain. Both viruses were propagated in human peripheral blood lymphocytes. Compounds were tested for there ability to block infection of the HOS cell line (expressing hCXCR4/hCCR5/hCD4/pHIV-LTR-luciferase) by either HIV-1 Ba-L or HIV-1 IIIB. Compound cytotoxicity was also examined in the absence of virus addition.

HOS HIV-1 Infectivity Assay Format

HOS cells (expressing hCXCR4/hCCR5/hCD4/pHIV-LTR-luciferase) were harvested and diluted in Dulbeccos modified Eagles media supplemented with 2% FCS and non-essential amino acid to a concentration of 60,000 cells/ml. The cells were plated into 96-well plates (100 ul per well) and the plates were placed in a tissue culture incubator (37° C.; 5% CO₂/95% air) for a period of 24 h.

Subsequently, 50 ul of the desired drug solution (4 times the final concentration) was added to each well and the plates were returned to the tissue culture incubator (37° C.; 5% CO₂/95% air) for 1 h. Following this incubation 50 ul of diluted virus was added to each well (approximately 2 million RLU per well of virus). The plates were returned to the tissue culture incubator (37° C.; 5% CO₂/95% air) and were incubated for a further 96 h.

Following this incubation the endpoint for the virally infected cultures was quantified following addition of Steady-Glo Luciferase assay system reagent (Promega, Madison, Wis.). Cell viability or non-infected cultures was measured using a CellTiter-Glo luminescent cell viability assay system (Promega, Madison, Wis.). All luminescent readouts are performed on a Topcount luminescence detector (Packard, Meridien, Conn.).

TABLE 1

| | | Functional | Fusion | | HOS |
|---|---|---|---|---|---|
| | | assay | assay | Cytotox | (3B) |
| Example | Structure | (pIC50) | (pIC50) | (pIC50) | (μM) |
| 6 | 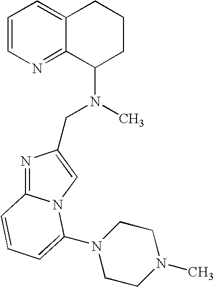 | 8.10 (n = 1) | 8.48 (n = 2) | <4.00 (n = 1) | 0.0063 (n = 1) |
| 7 | 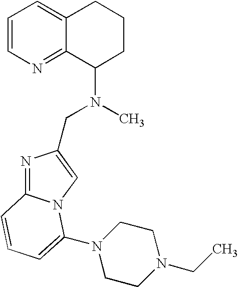 | 8.16 (n = 1) | 7.95 (n = 2) | <4.00 (n = 1) | 0.011 (n = 2) |
| 8 | 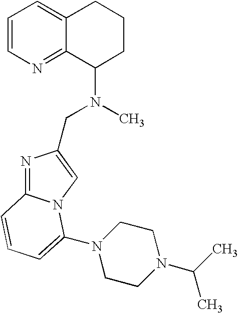 | 8.01 (n = 1) | 7.65 (n = 2) | <4.00 (n = 1) | 0.016 (n = 2) |
| 9 | 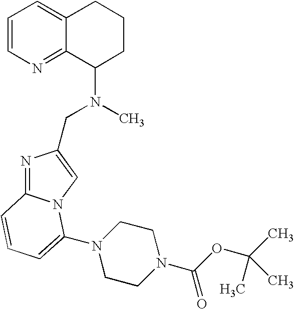 | 6.44 (n = 1) | 6.04 (n = 2) | <4.00 (n = 1) | 0.30 (n = 1) |
| 10 | 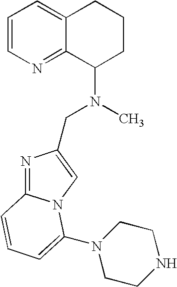 | 8.16 (n = 1) | 7.75 (n = 2) | <4.00 (n = 1) | 0.020 (n = 2) |
| 11 | 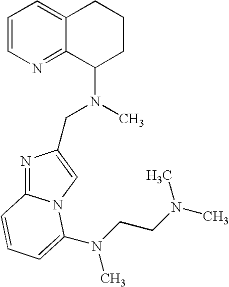 | 7.47 (n = 1) | 7.17 (n = 2) | <4.00 (n = 1) | 0.032 (n = 1) |
| 12 | 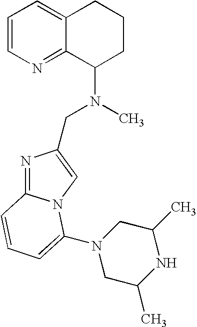 | 7.55 (n = 1) | 6.78 (n = 1) | <4.00 (n = 1) | 0.087 |
| 13 | 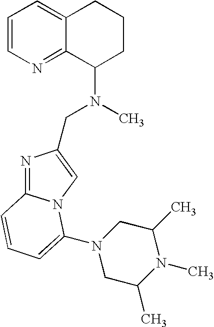 | 7.74 (n = 1) | 7.11 (n = 1) | <4.00 (n = 1) | 0.062 |
| 14 | 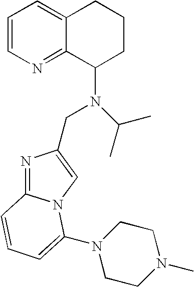 | 8.7 | 7.66 | <4 | 0.013 |
| 15 | 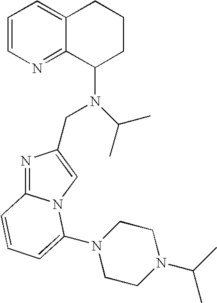 | 8.44 | 7.1 | <4 | 0.038 |
| 16 | 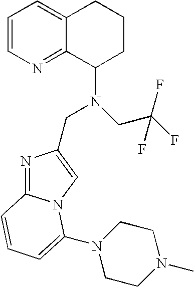 | 8.36 | 6.38 | <4 | 0.145 |
| 17 | 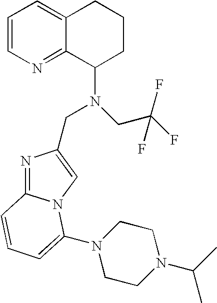 | 8.05 | 5.67 | <4 | 0.30 |
| 18 | 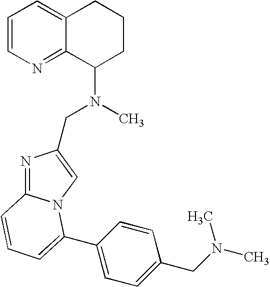 | 7.41 (n = 1) | 6.83 (n = 2) | <4.00 (n = 1) | 0.065 (n = 1) |
| 19 | 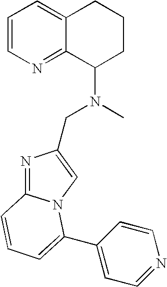 | 7.69 | 5.5 | <4 | 0.28 |

TABLE 2

| | | Activity |
|---|---|---|
| Example | Structure | Level* |
| 20 | 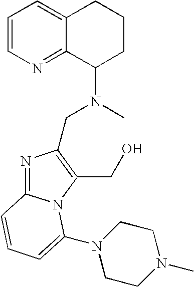 | A |
| 22 | 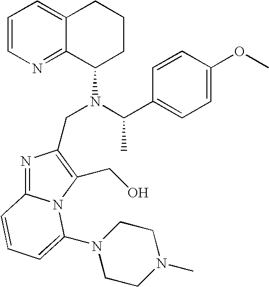 | A |
| 25 | 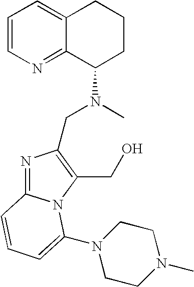 | A |
| 28 | 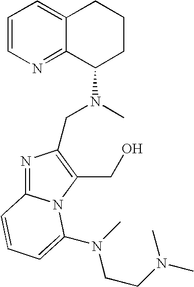 | A |
| 30 | 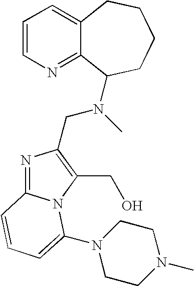 | A |
| 32 | 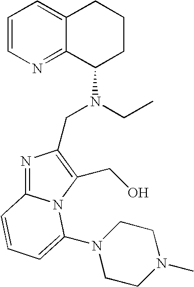 | A |
| 34 | 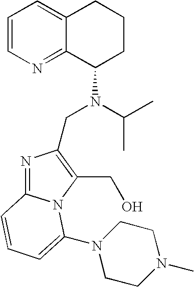 | A |
| 36 | 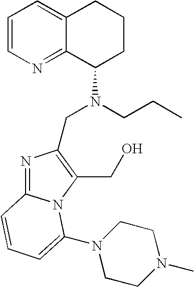 | A |
| 38 | 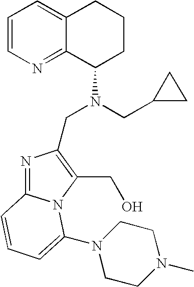 | A |
| 40 | 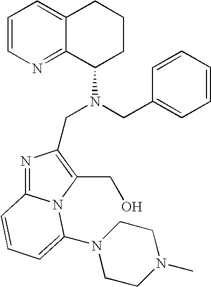 | A |
| 42 | 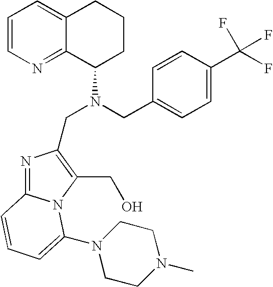 | A |
| 44 | 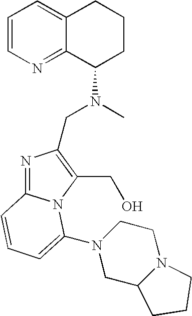 | A |
| 46 | 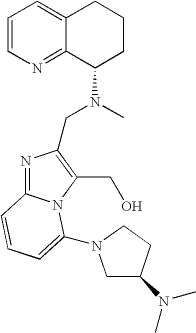 | A |
| 48 | 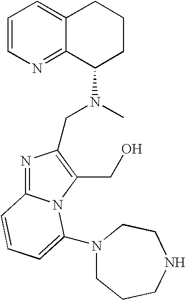 | A |
| 50 | 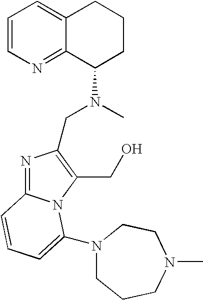 | A |
| 52 | 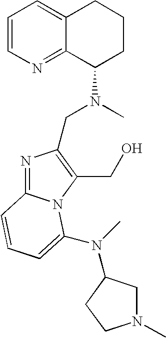 | A |
| 54 | 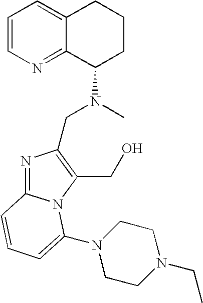 | A |
| 56 | 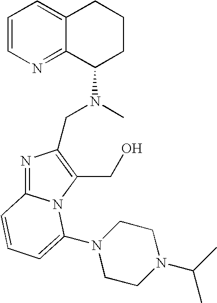 | A |
| 58 | 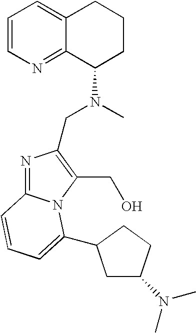 | A |
| 60 | 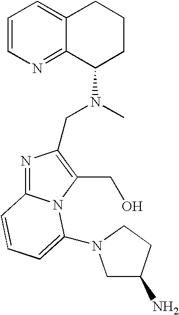 | A |
| 63 | 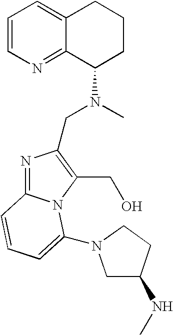 | A |
| 66 | 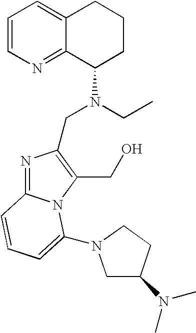 | A |
| 68 | 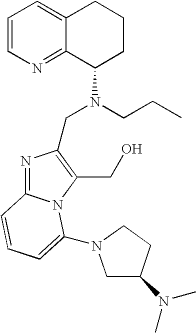 | A |
| 70 | 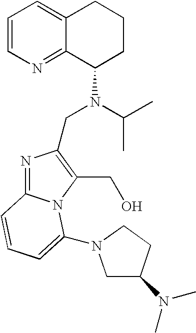 | A |
| 72 | 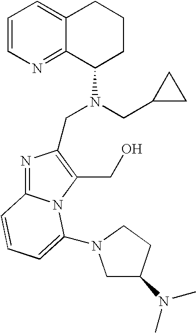 | A |
| 76 | 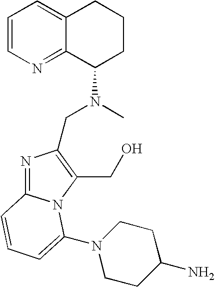 | A |
| 78 | 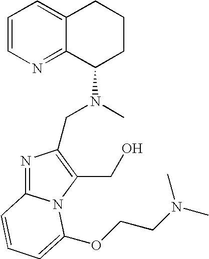 | A |
| 80 | 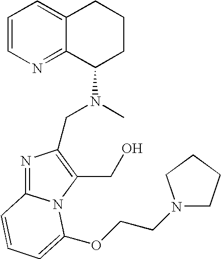 | A |
| 82 | 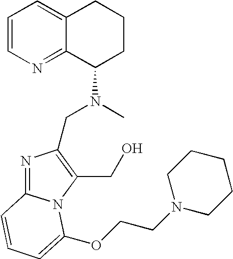 | A |
| 84 | 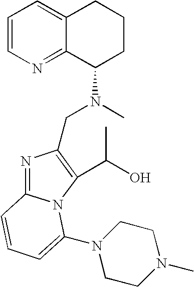 | A |
| 85 | 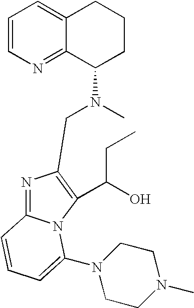 | A |
| 86 | 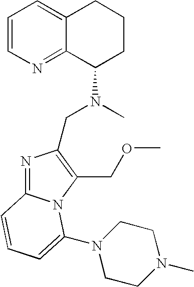 | A |
| 87 | 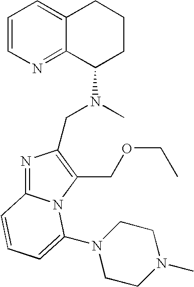 | A |
| 88 | 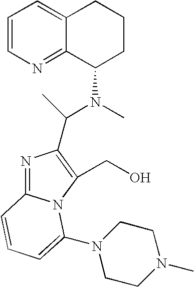 | A |
| 89A | 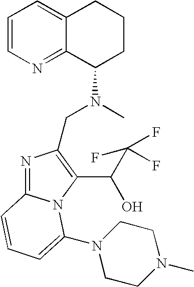 | A |
| 89B | 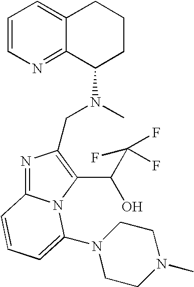 | B |

*“A” indicates an activity level of less than 100 nM in the HOS HIV anti-infectivity assay.
“B” indicates an activity level of between 100 nM to 500 nM in the HOS HIV anti-infectivity assay.
“C” indicates an activity level of between 500 nM and 10 μM in the HOS HIV anti-infectivity assay.

Compounds of the present invention demonstrate anti-HIV activity in the range of IC₅₀ of about 1 nM to about 50 μM. In one aspect of the invention, compounds of the present invention have anti-HIV activity in the range of up to about 100 nM. In another aspect of the invention, compounds of the present invention have anti-HIV activity in the range of from about 100 nM to about 500 nM. In another aspect of the invention, compounds of the present invention have anti-HIV activity in the range of from about 500 nM to 10 μM. In another aspect of the invention, compounds have anti-HIV activity in the range of from about 10 μM to about 50 μM.

Compounds of the present invention demonstrate desired potency. Antiviral activity is separated from cytotoxicity. Moreover, compounds of the present invention are believed to provide a desired pharamcokinetic profile. Also, compounds of the present invention are believed to provide a desired secondary biological profile. One aspect of the invention includes compounds of the present invention possessing desired physicochemical properties (e.g. desirable solid state properties).

Test compounds were employed in free or salt form.

All research complied with the principles of laboratory animal care (NIH publication No. 85-23, revised 1985) and GlaxoSmithKline policy on animal use.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of formula (I-G): 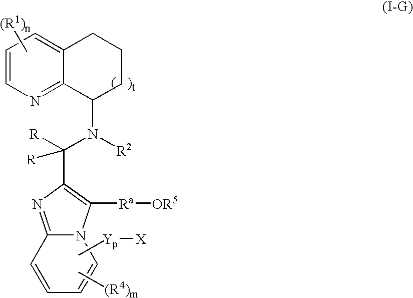
wherein:
t is 0, 1, or 2;
each R independently is H, alkyl, alkenyl, alkynyl, haloalkyl, cycloalkyl, —R^{a}Ay, —R^{a}OR¹⁰, or —R^{a}S(O)_{q}R¹⁰;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰—NR⁶R⁷, —R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R⁴ is selected from a group consisting of H, alkyl, haloalkyl, cycloalkyl, alkenyl, alkynyl, —R^{a}Ay, —R^{a}OR¹⁰, —R^{a}S(O)_{q}R⁵ or R^{a}cycloalkyl, and wherein R² is not substituted with amine or alkylamine;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, —R^{a}C(O)R¹⁰, —C(O)R¹⁰, —CO₂R¹⁰, —R^{a}CO₂R¹, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, —S(O)_{q}NR¹⁰—, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is alkylene optionally substituted with one or more of alkyl, oxo or hydroxyl, cycloalkylene optionally substituted with one or more of alkyl, oxo or hydroxyl, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a} OH, —R^{a}OR⁵, —R^{a}NR⁶R⁷, or —R^{a}Het
each of R⁶ and R⁷ independently are selected from H, alkyl, alkenyl, alkynyl, cycloalkyl, cycloalkenyl, —R^{a}cycloalkyl, —R^{a} OH, —R^{a}OR⁵, —R^{a}NR⁸R⁹, -Ay, -Het, —R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted heterocyclyl or heteroaryl group; or pharmaceutically acceptable salts or esters thereof.

2. The compound of claim 1 wherein -Het is optionally substituted with at least one of alkyl, alkoxy, hydroxyl, halogen, haloalkyl, cycloalkyl, cycloalkoxy, cyano, amide, amino, or alkylamino.

3. The compound of claim 1 wherein -Ay is optionally substituted with at least one of alkyl, alkoxy, hydroxyl, halogen, haloalkyl, cycloalkyl, cycloalkoxy, cyano, amide, amino, or alkylamino.

4. The compound of claim 1 wherein t is 1.

5. The compound of claim 1 wherein t is 2.

6. The compound of claim 1 wherein R is H, alkyl, cycloalkyl, or R^{a}OR¹⁰.

7. (canceled)

8. (canceled)

9. The compound of claim 1 wherein n is 0.

10. The compound of claim 1 wherein n is 1 and R¹ is halogen, haloalkyl, alkyl, OR¹⁰, NR⁶R⁷, CO₂R¹⁰, CONR⁶R⁷ or cyano.

11. The compound of claim 1 wherein R² is H, alkyl, haloalkyl, R^{a}OR⁵ or R^{a}cycloalkyl.

12. (canceled)

13. (canceled)

14. The compound of claim 1 wherein R² is R^{a}Ay or R^{a}cycloalkyl.

15. The compound of claim 1 wherein R^{a} is alkylene optionally substituted with C₁-C₆alkyl and R⁵ is H, alkyl or cycloalkyl.

16. The compound of claim 1 wherein R^{a} is methylene (—CH₂—) optionally substituted with C₁-C₆alkyl and R⁵ is H, alkyl or cycloalkyl.

17. The compound of claim 1 wherein R^{a} is methylene (—CH₂—) and R⁵ is H.

18. The compound of claim 1 wherein m is 0.

19. (canceled)

20. The compound of claim 1 wherein m is 1.

21. The compound of claim 20 wherein R⁴ is halogen, haloalkyl, alkyl, OR¹⁰, NR⁶R⁷, CO₂R¹⁰, CONR⁶R⁷ or cyano.

22. The compound of claim 1 wherein p is 0 and X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂.

23. The compound of claim 22 wherein X is —R^{a}N(R¹⁰)₂, -Het, —R^{a}Het, -HetN(R¹⁰)₂, —R^{a}HetN(R¹⁰)₂, or -HetR^{a}N(R¹⁰)₂.

24. The compound of claim 23 wherein X is —R^{a}N(R¹⁰)₂, -Het, —R^{a}Het, or -HetN(R¹⁰)₂.

25. The compound of claim 1 wherein
p is 1;
Y is —N(R¹⁰)—, —O—, —S—, —C(O)NR¹⁰—, —NR¹⁰C(O)—, or —S(O)_{q}NR¹⁰—; and
X is —R^{a}N(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, or -HetR^{a}N(R¹⁰)₂.

26. The compound of claim 25 wherein
Y is —N(R¹⁰)—, —O—, —C(O)NR¹⁰—, or —NR¹⁰C(O)—; and
X is —R^{a}N(R¹⁰)₂, -Het, —R^{a}Het, or -HetN(R¹⁰)₂.

27. The compound of claim 1 wherein t is 1 or 2; R is H or alkyl; R² is H, alkyl, R^{a}cycloalkyl or cycloalkyl; n is 0; m is 0; and wherein with respect to —R^{a}OR⁵, R^{a} is alkylene optionally substituted with C₁-C₆alkyl and R⁵ is H, alkyl or cycloalkyl.

28. The compound of claim 27 wherein p is 0 and X is -Het or -HetN(R¹⁰)₂, R¹⁰ is H or alkyl and -Het is unsubstituted or substituted with C₁-C₆ alkyl or C₃-C₈ cycloalkyl.

29. The compound of claim 27 wherein —R^{a}OR⁵ is —CH₂OH.

30. The compound of claim 27 wherein p is 1; Y is —N(R¹⁰)—, —O—, —CONR¹⁰—, or —NR¹⁰CO—; X is -Het or -HetN(R¹⁰)₂, and R¹⁰ is H or alkyl and Het is unsubstituted or substituted with C₁-C₆ alkyl or C₃-C₈ cycloalkyl.

31. The compound of claim 30 wherein Y is —N(R¹⁰)— or —O— and X is -Het.

32. The compound of claim 1 wherein p is 0; X is -HetN(R¹⁰)₂;
and R¹⁰ is H or alkyl.

33. The compound of claim 1 wherein p is 1 and Y is —N(R¹⁰)—, —O—, —C(O)NR¹⁰—, or —NR¹⁰C(O)—; X is -Het or -HetN(R¹⁰)₂, and Het is unsubstituted or substituted with C₁-C₆ alkyl or C₃-C₈ cycloalkyl.

34. The compound of claim 1 wherein the substituent —Yₚ—X is located on the depicted imidazopyridine ring as in formula (I-G′):

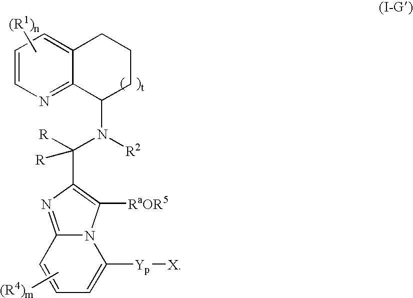

35. The compound of claim 1 wherein -Het is piperidine, piperazine, azetidine, pyrrolidine, imidazole, or pyridine.

36. The compound of claim 34 where p is 0 and X is -Het.

37. (canceled)

38. The compound of claim 36 wherein -Het is unsubstituted or substituted with one or more C₁-C₆ alkyl or C₃-C₈ cycloalkyl.

39. A compound selected from the group consisting of:
(5-(4-Methyl-1-piperazinyl)-2-{[methyl(5,6,7,8-tetrahydro-8-quinolinyl)amino]methyl}imidazo[1,2-a]pyridin-3-yl)methanol;
[2-({{(1S)-1-[4-(Methyloxy)phenyl]ethyl}[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({methyl[(8S)-5,6,7,8-tetrahydro-8-quinolinyl]amino}methyl)imidazo[1,2-a]pyridin-3-yl]methanol;
[5-[[2-(Dimethylamino)ethyl](methyl)amino]-2-({methyl[(8S)-5,6,7,8-tetrahydro-8-quinolinyl]amino}methyl)imidazo[1,2-a]pyridin-3-yl]methanol;
(5-(4-M ethyl-1-piperazinyl)-2-{[methyl(6,7,8,9-tetrahydro-5H-cyclohepta[b]pyridin-9-yl)amino]methyl}imidazo[1,2-a]pyridin-3-yl)methanol;
[2-({Ethyl[(8S)-5,6,7,8-tetrahydro-8-quinolinyl]amino}methyl)-5-(4-methyl-1-piperazinyl)imidazo[1,2-a]pyridin-3-yl]methanol;
[2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({propyl[(8S)-5,6,7,8-tetrahydro-8-quinolinyl]amino}methyl)imidazo[1,2-a]pyridin-3-yl]methanol;
[2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({(phenylmethyl)[(8S)-5,6,7,8-tetrahydro-8-quinolinyl]amino}methyl)imidazo[1,2-a]pyridin-3-yl]methanol;
{5-(4-Methyl-1-piperazinyl)-2-[((8S)-5,6,7,8-tetrahydro-8-quinolinyl{[4-(trifluoromethyl)phenyl]methyl}amino)methyl]imidazo[1,2-a]pyridin-3-yl}methanol;
[5-(Hexahydropyrrolo[1,2-a]pyrazin-2(1H)-yl)-2-({methyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-(Hexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol;
[5-(4-Methylhexahydro-1H-1,4-diazepin-1-yl)-2-({methyl[(8S)-5,6,7,8-tetrahydro-8-quinolinyl]amino}methyl)imidazo[1,2-a]pyridin-3-yl]methanol;
[5-[Methyl(1-methyl-3-pyrrolidinyl)amino]-2-({methyl[(8S)-5,6,7,8-tetrahydro-8-quinolinyl]amino}methyl imidazo[1,2-a]pyridin-3-yl]methanol;
[5-(4-Ethyl-1-piperazinyl)-2-({methyl[(8S)-5,6,7,8-tetrahydro-8-quinolinyl]amino}methyl)imidazo[1,2-a]pyridin-3-yl]methanol;
[5-[4-(1-Methylethyl)-1-piperazinyl]-2-({methyl[(8S)-5,6,7,8-tetrahydro-8-quinolinyl]amino}methyl)imidazo[1,2-a]pyridin-3-yl]methanol;
[5-[(3S)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-Amino-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Methylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol;
{2-({(Cyclopropylmethyl)[(8S)-5,6,7,8-tetrahydro-8-quinolinyl]amino}methyl)-5-[(3R)-3-(dimethylamino)-1-pyrrolidinyl]imidazo[1,2-a]pyridin-3-yl}methanol;
1-[5-(4-Methyl-1-piperazinyl)-2-({methyl[(8S)-5,6,7,8-tetrahydro-8-quinolinyl]amino}methyl)imidazo[1,2-a]pyridin-3-yl]ethanol;
1-[5-(4-M ethyl-1-piperazinyl)-2-({methyl[(8S)-5,6,7,8-tetrahydro-8-quinolinyl]amino}methyl)imidazo[1,2-a]pyridin-3-yl]-1-propanol;
(8S)—N-Methyl-N-{[3-[(methyloxy)methyl]-5-(4-methyl-1-piperazinyl)imidazo[1,2-a]pyridin-2-yl]methyl}-5,6,7,8-tetrahydro-8-quinolinamine;
(8S)—N-{[3-[(Ethyloxy)methyl]-5-(4-methyl-1-piperazinyl)imidazo[1,2-a]pyridin-2-yl]methyl}-N-methyl-5,6,7,8-tetrahydro-8-quinolinamine;
[5-(4-M ethyl-1-piperazinyl)-2-(1-{methyl[(8S)-5,6,7,8-tetrahydro-8-quinolinyl]amino}ethyl)imidazo[1,2-a]pyridin-3-yl]methanol;
2,2,2-Trifluoro-1-(5-(4-methyl-1-piperazinyl)-2-{([methyl(5,6,7,8-tetrahydro-8-quinolinyl)amino]methyl}imidazo[1,2-a]pyridin-3-yl)ethanol; and pharmaceutically acceptable salts and esters thereof.

40. A compound selected from the group consisting of:
(5-(4-Methyl-1-piperazinyl)-2-{[methyl(5,6,7,8-tetrahydro-8-quinolinyl)amino]methyl}imidazo[1,2-a]pyridin-3-yl)methanol;
[5-(4-Methyl-1-piperazinyl)-2-({methyl[(8S)-5,6,7,8-tetrahydro-8-quinolinyl]amino}methyl)imidazo[1,2-a]pyridin-3-yl]methanol;
[2-({Ethyl[(8S)-5,6,7,8-tetrahydro-8-quinolinyl]amino}methyl)-5-(4-methyl-1-piperazinyl)imidazo[1,2-a]pyridin-3-yl]methanol;
[2-({(1-Methylethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Methyl-1-piperazinyl)-2-({propyl[(8S)-5,6,7,8-tetrahydro-8-quinolinyl]amino}methyl)imidazo[1,2-a]pyridin-3-yl]methanol;
[2-({(Cyclopropylmethyl)[(8S)-5,6,7,8-tetrahydro-8-quinolinyl]amino}methyl)-5-(4-methyl-1-piperazinyl)imidazo[1,2-a]pyridin-3-yl]methanol;
[5-(4-Ethyl-1-piperazinyl)-2-({methyl[(8S)-5,6,7,8-tetrahydro-8-quinolinyl]amino}methyl)imidazo[1,2-a]pyridin-3-yl]methanol;
[5-[4-(1-Methylethyl)-1-piperazinyl]-2-({methyl[(8S)-5,6,7,8-tetrahydro-8-quinolinyl]amino}methyl)imidazo[1,2-a]pyridin-3-yl]methanol; and pharmaceutically acceptable salts or esters thereof.

41. A compound selected from the group consisting of:
[5-[(3R)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[Methyl(1-methyl-3-pyrrolidinyl)amino]-2-({methyl[(8S)-5,6,7,8-tetrahydro-8-quinolinyl]amino}methyl)imidazo[1,2-a]pyridin-3-yl]methanol;
[5-[(3S)-3-(Dimethylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-Amino-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Methylamino)-1-pyrrolidinyl]-2-({methyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({ethyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({propyl[(8S)-5,6,7,8-tetrahydro-8-quinolinyl]amino}methyl)imidazo[1,2-a]pyridin-3-yl]methanol;
[5-[(3R)-3-(Dimethylamino)-1-pyrrolidinyl]-2-({(1-methylethyl)[(8S)-5,6,7,8-tetrahydro-8-quinolinyl]amino}methyl)imidazo[1,2-a]pyridin-3-yl]methanol;
{2-({(Cyclopropylmethyl)[(8S)-5,6,7,8-tetrahydro-8-quinolinyl]amino}methyl)-5-[(3R)-3-(dimethylamino)-1-pyrrolidinyl]imidazo[1,2-a]pyridin-3-yl}methanol;
[5-(4-Amino-1-piperidinyl)-2-({methyl[(8S)-5,6,7,8-tetrahydro-8-quinolinyl]amino}methyl)imidazo[1,2-a]pyridin-3-yl]methanol; and pharmaceutically acceptable salts or esters thereof.

42. The compound of claim 40 wherein said compound is in solid form.

43. (canceled)

44. A pharmaceutical composition comprising a compound according to claim 1, and a pharmaceutically acceptable carrier.

45. A composition according to claim 44, wherein said composition comprises at least one additional therapeutic agent selected from the group consisting of nucleotide reverse transcriptase inhibitors; non-nucleotide reverse transcriptase; protease inhibitors; entry inhibitors; Integrase inhibitors; budding inhibitors; CXCR4; and CCR5.

46. A compound according to claim 1 for use as an active therapeutic substance.

47. A compound according to claim 1 for use in the treatment or prophylaxis of diseases and conditions caused by inappropriate activity of CXCR4.

48. A compound according to claim 1 for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers.

49. The compound of claim 48 wherein the condition or disease is HIV infection, rheumatoid arthritis, inflammation, or cancer.

50. The compound of claim 48 wherein the condition or disease is HIV infection.

51. Use of a compound according to claim 1 in the manufacture of a medicament for use in the treatment or prophylaxis of a condition or disease modulated by a chemokine receptor.

52. Use of a compound according to claim 50 wherein the chemokine receptor is CXCR4.

53. Use of a compound according to claim 1 in the manufacture of a medicament for use in the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers.

54. Use of a compound as in claim 53 wherein the condition or disorder is HIV infection, rheumatoid arthritis, inflammation, or cancer.

55. Use of a compound as in claim 53 wherein the condition or disorder is HIV infection.

56. A method for the treatment or prophylaxis of a condition or disease modulated by a chemokine receptor comprising the administration of a compound of claim 1.

57. The method of claim 56 wherein the chemokine receptor is CXCR4

58. A method for the treatment or prophylaxis of HIV infection, diseases associated with hematopoiesis, controlling the side effects of chemotherapy, enhancing the success of bone marrow transplantation, enhancing wound healing and burn treatment, combating bacterial infections in leukemia, inflammation, inflammatory or allergic diseases, asthma, allergic rhinitis, hypersensitivity lung diseases, hypersensitivity pneumonitis, eosinophilic pneumonitis, delayed-type hypersensitivity, interstitial lung disease (ILD), idiopathic pulmonary fibrosis, systemic lupus erythematosus, ankylosing spondylitis, systemic sclerosis, Sjogren's syndrome, polymyositis or dermatomyositis, systemic anaphylaxis or hypersensitivity responses, drug allergies, insect sting allergies, autoimmune diseases, rheumatoid arthritis, psoriatic arthritis, systemic lupus erythematosus, myastenia gravis, juvenile onset diabetes, glomerulonephritis, autoimmune throiditis, graft rejection, allograft rejection, graft-versus-host disease, inflammatory bowel diseases, Crohn's disease, ulcerative colitus, spondylo-arthropathies, scleroderma, psoriasis, T-cell-mediated psoriasis, inflammatory dermatoses, dermatitis, eczema, atopic dermatitis, allergic contact dermatitis, urticaria, vasculitis, necrotizing, cutaneous, hypersensitivity vasculitis, eoosinophilic myotis, eosinophilic fasciitis, and brain, breast, prostate, lung, or haematopoetic tissue cancers comprising the administration of a compound according to claim 1.

59. A method for the treatment or prophylaxis of HIV infection rheumatoid arthritis, inflammation, or cancer comprising the administration of a compound according to claim 1.

60. A method for the treatment or prophylaxis of HIV infection comprising the administration of a compound according to claim 1.

61. A method of treatment or prevention of a viral infection in a human comprising administering to said human a composition comprising a compound according to claim 1 to and another therapeutic agent.

62. A method according to claim 61, wherein said therapeutic agent is selected from the group consisting of nucleotide reverse transcriptase inhibitors; non-nucleotide reverse transcriptase inhibitors; protease inhibitors; entry inhibitors; Integrase inhibitors; budding inhibitors; CXCR4; and CCR5 inhibitors.

63. The process of preparing a compound of formula (I-G) 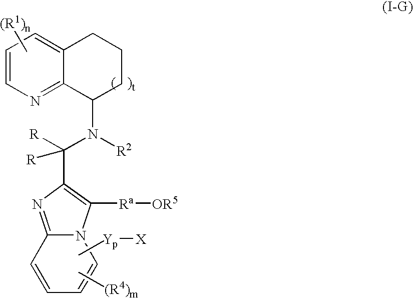
wherein t is 1; each R is H;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, alkyl, haloalkyl, cycloalkyl, alkenyl, alkynyl —R^{a}Ay, —R^{a}OR⁵, —R^{a}S(O)_{q}R⁵ or R^{a}cycloalkyl;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, S(O)_{q}NR¹⁰—, or —NR¹⁰S(O)_{q}—;
X is —N(R¹⁰)₂, —R^{a}N(R¹⁰)₂, -AyN(R¹⁰)₂, R^{a}AyN(R¹)₂, -AyR^{a}N(R¹⁰)₂—R^{a}AyR^{a}N(R)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is alkylene optionally substituted with one or more of alkyl, oxo or hydroxyl, cycloalkylene optionally substituted with one or more of alkyl, oxo or hydroxyl, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het;
each of R⁶ and R⁷ independently are selected from H, alkyl, alkenyl, alkynyl, cycloalkyl, cycloalkenyl, —R^{a}cycloalkyl, —R^{a} OH, —R^{a}OR⁵, —R^{a}NR⁸R⁹, -Ay, -Het, —R^{a}Ay —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted heterocyclyl or heteroaryl group,
comprising the step of reacting a compound of formula (II) 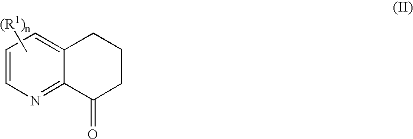
wherein R¹ and n is as defined with respect to formula (I-G) with compound of formula (IV) 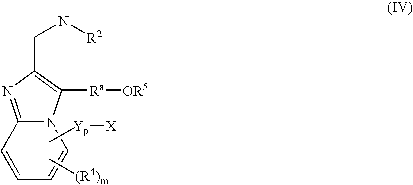
wherein R², R^{a}, R⁴, R⁵, Y, X, p and m are as defined with respect to formula (I-G) under reductive amination conditions to form a compound of formula (I-G).

64. The process of preparing a compound of formula (I-G) 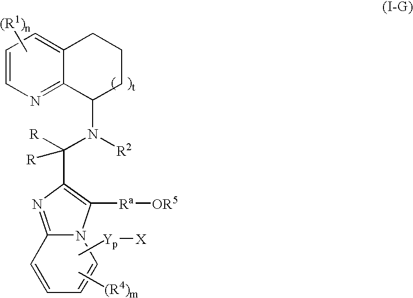
wherein t is 1; each R is H;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, alkyl, haloalkyl, cycloalkyl, alkenyl, alkynyl, —R^{a}Ay, —R^{a}OR⁵, —R^{a}S(O)_{q}R⁵ or R^{a}cycloalkyl;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, —R^{a}C(O)R¹⁰, —C(O)R¹⁰—CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, S(O)_{q}NR¹⁰—, or —NR¹⁰S(O)_{q}—;
X is —N(R¹⁰)₂, R^{a}N(R¹⁰)₂, -AyN(R¹⁰)₂, —R^{a}AyN(R¹⁰)₂, -AyR^{a}N(R¹⁰)₂, —R AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is alkylene optionally substituted with one or more of alkyl, oxo or hydroxyl, cycloalkylene optionally substituted with one or more of alkyl, oxo or hydroxyl, alkenylene, cycloalkenylene, or alkynylene;
each R¹⁰ independently is H, alkyl, cycloalkyl, alkenyl, alkynyl, cycloalkenyl, R^{a}cycloalkyl, —R^{a} OH, —R^{a}OR⁵, —R^{a}NR⁶R⁷, or R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het, —R^{a}Ay, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted heterocyclyl or heteroaryl group;
comprising the step of reacting a compound of formula (III) 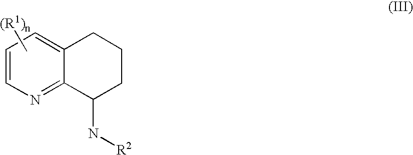
wherein R¹, R² and n are as defined with respect to formula (I-G) with compound of formula (V) 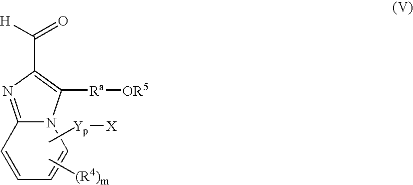
wherein R^{a}, R⁴, R⁵, Y, X, p and m are as defined with respect to formula (I-G) under reductive amination conditions to form a compound of formula (I-G).

65. The process of preparing a compound of formula (I-G) 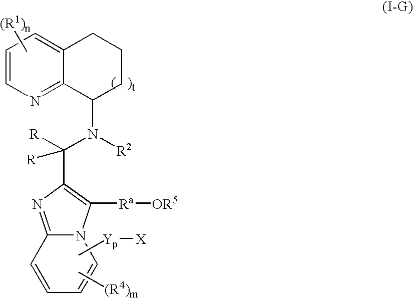
wherein t is 1; each R is H;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, alkyl, haloalkyl, cycloalkyl, alkenyl, alkynyl, —R^{a}Ay, —R^{a}OR⁵, —R^{a}S(O)_{q}R⁵ or R^{a}cycloalkyl;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, S(O)_{q}NR¹, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is alkylene optionally substituted with one or more of alkyl, oxo or hydroxyl, cycloalkylene optionally substituted with one or more of alkyl, oxo or hydroxyl, alkenylene, cycloalkenylene, or alkynylene;
each R¹⁰ independently is H, alkyl, cycloalkyl, alkenyl, alkynyl, cycloalkenyl, R^{a}cycloalkyl, —R^{a}OH, —RₐOR⁵, —R^{a}NR⁶R⁷, or —R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het, —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted heterocyclyl or heteroaryl group;
comprising the step of reacting a compound of formula (III) 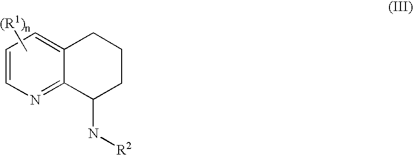
wherein R¹, R² and n are as defined with respect to formula (I-G) with compound of formula (VI) 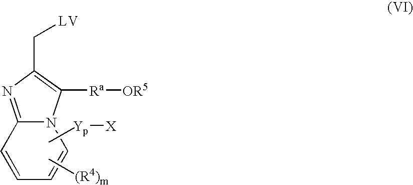
wherein R^{a}, R⁴, R⁵, Y, X, p and m are as defined with respect to formula (I-G) and LV is a leaving group to form compound of formula (I-G).

66. The process of preparing a compound of formula (I-E) 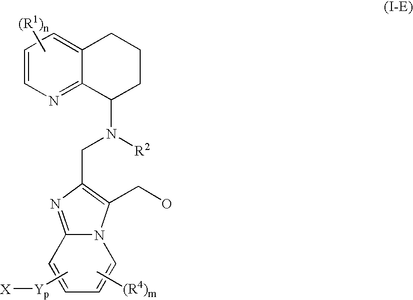
wherein each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR₁₁, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, alkyl, haloalkyl, cycloalkyl, alkenyl, alkynyl, —R^{a}Ay, —R^{a}OR⁵, —R^{a}S(O)_{q}R⁵ or R^{a}cycloalkyl;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰), —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, S(O)_{q}NR¹⁰, or —NR¹⁰S(O)_{q}—;
X is —N(R¹⁰)₂, —R^{a}N(R¹⁰)₂, -AyN(R¹⁰)₂, —R^{a}AyN(R¹⁰)₂, -AyR^{a}N(R¹⁰)₂—R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, —R^{a}HetR^{a}N(R¹⁰)₂, -HetR^{a}Ay, or -HetR^{a}Het;
each R^{a} independently is alkylene optionally substituted with one or more of alkyl, oxo or hydroxyl, cycloalkylene optionally substituted with one or more of alkyl, oxo or hydroxyl, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or —R^{a}Het;
each of R⁶ and R⁷ independently are selected from H, alkyl, alkenyl, alkynyl, cycloalkyl, cycloalkenyl, R^{a}cycloalkyl, —R^{a} OH, —R^{a}OR⁵, —R^{a}NR⁵R⁹, -Ay, -Het, —R^{a}Ay —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted heterocyclyl or heteroaryl group;
comprising the step of treating a compound of formula (X-B) 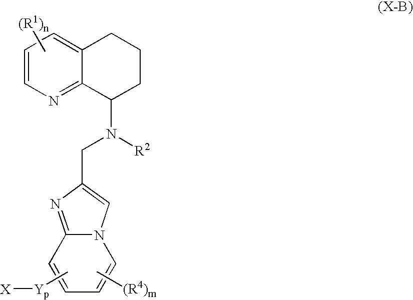
wherein R², R¹, R⁴, Y, X, n, p and m are as defined with respect to formula (I-E), with formaldehyde under acidic conditions to form compound of formula (I-E).

67. The process of preparing a compound of formula (I-G) 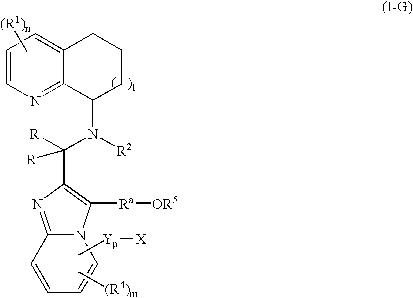
wherein t is 1; each R is H; —R^{a}OR⁵ is —CH₂OH;
each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, —R^{a}C(O)R¹⁰, —C(O)R¹⁰, —CO₂R¹⁰-R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, alkyl, haloalkyl, cycloalkyl, alkenyl, alkynyl, —R^{a}Ay, —R^{a}OR⁵, —R^{a}S(O)_{q}R⁵ or R^{a}cycloalkyl;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
p is 0 or 1;
Y is —NR¹⁰—, —O—, —C(O)NR¹⁰—, —NR¹⁰C(O)—, —C(O)—, —C(O)O—, —NR¹⁰C(O)N(R¹⁰)—, —S(O)_{q}—, S(O)_{q}NR¹⁰—, or —NR¹⁰S(O)_{q};
X is —N(R¹⁰)₂, —R^{a}N(R¹⁰)₂, -AyN(R¹⁰)₂, —R^{a}AyN(R¹⁰)₂, -AyR^{a}N(R¹⁰)₂, —R^{a}AyR^{a}N(R¹⁰)₂, -Het, —R^{a}Het, -HetN(R¹⁰)₂, —R^{a}HetN(R¹⁰)₂, -HetR^{a}N(R¹⁰)₂, R^{a}HetR^{a}N(R¹⁰)₂, -HetR^{a}Ay, or -HetR^{a}Het;
each R^{a} independently is alkylene optionally substituted with one or more of alkyl, oxo or hydroxyl, cycloalkylene optionally substituted with one or more of alkyl, oxo or hydroxyl, alkenylene, cycloalkenylene, or alkynylene;
each R¹⁰ independently is H, alkyl, cycloalkyl, alkenyl, alkynyl, cycloalkenyl, R^{a}cycloalkyl, —R^{a} OH, —R^{a}OR⁵, —R^{a}NR⁶R⁷, or —R^{a}Het
each of R⁶ and R⁷ independently are selected from H, alkyl, alkenyl, alkynyl, cycloalkyl, cycloalkenyl, —R^{a}cycloalkyl, —R^{a}OH, R^{a}OR⁵, —R^{a}NR⁸R⁹, -Ay, -Het, —R^{a}Ay, —R²Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted heterocyclyl or heteroaryl group,
comprising the step of formylating a compound of formula (X-B) 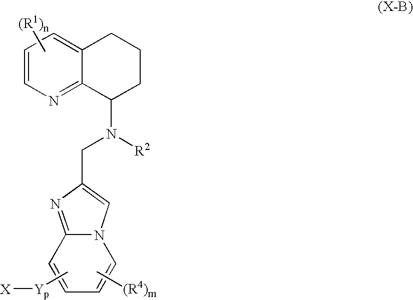
wherein R¹, R², R⁴, Y, X, n, p and m are as defined with respect to formula (I-G) followed by reduction to form a compound of formula (I-G).

68. The process of preparing a compound of formula (I-B) 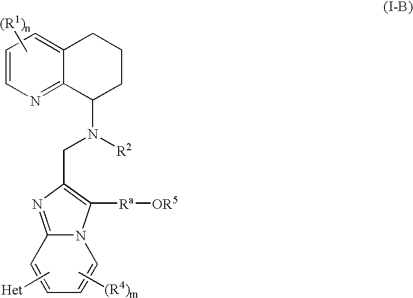
wherein each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, alkyl, haloalkyl, cycloalkyl, alkenyl, alkynyl, —R^{a}Ay, —R^{a}OR⁵, —R^{a}S(O)_{q}R⁵ or R^{a}cycloalkyl;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, R^{a}NR⁶R⁷, R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
each R^{a} independently is alkylene optionally substituted with one or more of alkyl, oxo or hydroxyl, cycloalkylene optionally substituted with one or more of alkyl, oxo or hydroxyl, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a} OH, —R^{a}OR⁵, —R^{a}NR⁶R⁷, or —R^{a}Het,
each of R⁶ and R⁷ independently are selected from H, alkyl, alkenyl, alkynyl, cycloalkyl, cycloalkenyl, —R^{a}cycloalkyl, —R^{a} OH, —R^{a}OR⁵, —R⁸NR⁸R⁹, -Ay, -Het, -—R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted heterocyclyl or heteroaryl group;
comprising the step of coupling a compound of formula (X) 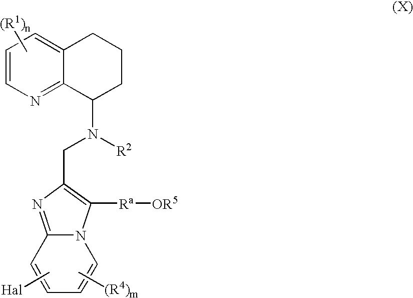
wherein R¹, R², R^{a}, R⁴, R⁵, n and m are as defined with respect to formula (I-B), with a compound of formula (XI-B)
Het-B(OH)₂ (XI-B)
in the presence of catalyst to form a compound of formula (I-B).

69. The process of preparing a compound of formula (I-C) 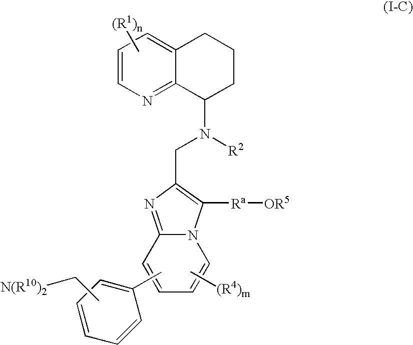
wherein each R¹ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR¹⁰, —NR⁶R⁷, —R^{A}NR⁶R⁷, —R^{a}C(O)R¹⁰, —C(O)R¹⁰, —CO₂R¹⁰, —R^{a}CO₂R¹⁰—C(O)NR⁶R₇, —C(O)Ay, —C(O)Het, —S(O)₂NR⁶R⁷, —S(O)_{q}R¹⁰, —S(O)_{q}Ay, cyano, nitro, or azido;
n is 0, 1, or 2;
R² is selected from a group consisting of H, alkyl, haloalkyl, cycloalkyl, alkenyl, alkynyl, —R^{a}Ay, —R^{a}OR⁵, —R^{a}S(O)_{q}R⁵ or R^{a}cycloalkyl;
each R⁴ independently is halogen, haloalkyl, alkyl, alkenyl, alkynyl, cycloalkyl, cycloalkenyl, -Ay, —NHAy, -Het, —NHHet, —OR¹⁰, —OAy, —OHet, —R^{a}OR⁵, —NR⁶R⁷, R^{a}NR⁶R⁷, —R^{a}C(O)R¹⁰, —C(O)R¹⁰, —CO₂R¹⁰, —R^{a}CO₂R¹⁰, —C(O)NR⁶R⁷, —C(O)Ay, —C(O)Het, —S(O)₂NR⁶R⁷, —S(O)_{q}R¹⁰, —S(O)_{q}Ay, cyano, nitro, or azido;
m is 0, 1, or 2;
each R⁵ independently is H, alkyl, alkenyl, alkynyl, cycloalkyl, or -Ay;
each R^{a} independently is alkylene optionally substituted with one or more of alkyl, oxo or hydroxyl, cycloalkylene optionally substituted with one or more of alkyl, oxo or hydroxyl, alkenylene, cycloalkenylene, or alkynylene;
each R¹⁰ independently is H, alkyl, cycloalkyl, alkenyl, alkynyl, cycloalkenyl, —R^{a}cycloalkyl, —R^{a}OH, —R^{a}OR⁵, —R^{a}NR⁶R⁷, or R^{a}Het;
each of R⁶ and R⁷ independently are selected from H, alkyl, alkenyl, alkynyl, cycloalkyl, cycloalkenyl, —R^{a}cycloalkyl, —R^{a}OH, —R^{a}OR⁵, —R^{a}NR⁸R⁹, -Ay, -Het, —R^{a}Ay —R^{a}Het, or —S(O)_{q}R⁵;
each of R⁸ and R⁹ independently are selected from H or alkyl;
each q independently is 0, 1, or 2;
each Ay independently represents an optionally substituted aryl group; and
each Het independently represents an optionally substituted heterocyclyl or heteroaryl group,
comprising the steps of coupling a compound of formula (X) 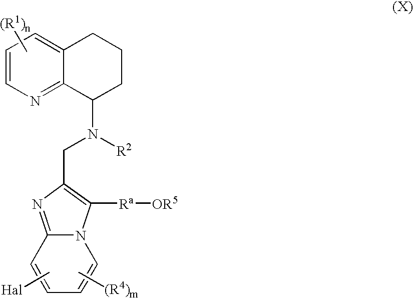
wherein R¹, R², R^{a}, R⁴, R⁵, n and m are as defined with respect to formula (I-C) with a compound of formula (XIII) 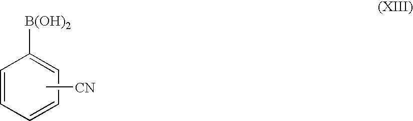
in the presence of a catalyst to form a compound of formula (XII) 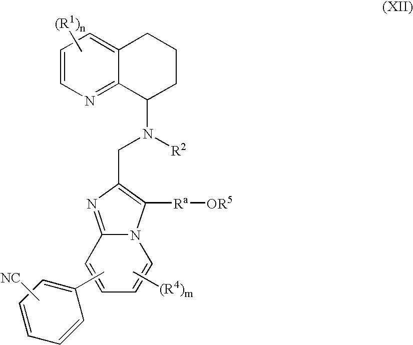
wherein R¹, R², R^{a}, R⁴, R⁵, n and m are as defined with respect to formula (I-C) and following said coupling with reduction of the compound of formula (XII) to form a compound of formula (I-C).

70. The process of preparing a compound of formula (I-F) 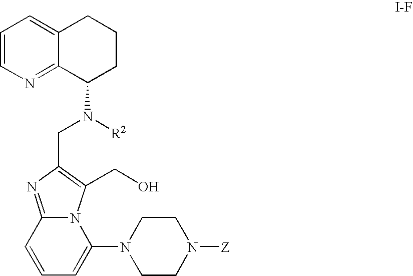
wherein R² is selected from a group consisting of H, alkyl, haloalkyl, cycloalkyl, alkenyl, alkynyl, —R^{a}Ay, —R^{a}OR⁵, —R^{a}S(O)_{q}R⁵; and Z is C₁-C₆ alkyl or C₃-C₈cycloalkyl;
comprising the steps of treating a compound of formula (XXXI-B) 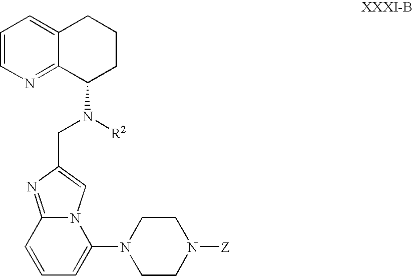
wherein R² and Z are as defined with respect to formula (I-F) with formaldehyde under acidic conditions to form a compound of formula (I-F).

71. The process of preparing a compound of formula (I-F) 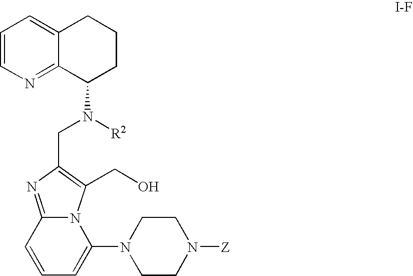
wherein R² is selected from a group consisting of H, alkyl, haloalkyl, cycloalkyl, alkenyl, alkynyl, —R^{a}Ay, —R^{a}OR⁵, —R^{a}S(O)_{q}R⁵; and Z is C₁-C₆ alkyl or C₃-C₈cycloalkyl;
comprising the steps of formylating a compound of formula (XXXI-B) 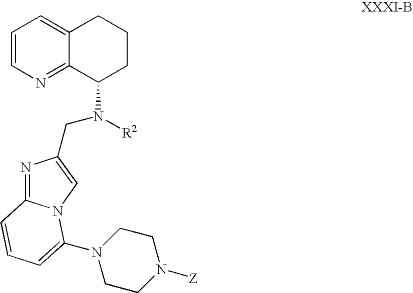
wherein R² and Z are as defined with respect to formula (I-F) followed by reduction to form a compound of formula (I-F).
